SEQUENCE LISTING (52 sequences)

SEQ ID NO: 1 - protein, 15 aa, Streptococcus pneumoniae
GWQKNDTGYWYVHSD

SEQ ID NO: 2 - protein, 21 aa, Streptococcus pneumoniae
GSYPKDKFEKINGTWYYFDSS

SEQ ID NO: 3 - protein, 22 aa, Streptococcus pneumoniae
GYMLADRWRKHTDGNWYWFDNS

SEQ ID NO: 4 - protein, 20 aa, Streptococcus pneumoniae
GEMATGWKKIADKWYYFNEE

SEQ ID NO: 5 - protein, 21 aa, Streptococcus pneumoniae
GAMKTGWVKYKDTWYYLDAKE

SEQ ID NO: 6 - protein, 23 aa, Streptococcus pneumoniae
GAMVSNAFIQSADGTGWYYLKPD

SEQ ID NO: 7 - protein, 142 aa, Streptococcus pneumoniae
GWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPEFTVEPDGLITVK

SEQ ID NO: 8 - protein, 112 aa, Streptococcus pneumoniae
YVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPD

SEQ ID NO: 9 - DNA, 45 bp, Streptococcus pneumoniae
ggctggcagaagaatgacactggctactggtacgtacattcagac

SEQ ID NO: 10 - DNA, 63 bp, Streptococcus pneumoniae
ggctcttatccaaaagacaagtttgagaaaatcaatggcacttggtactactttgacagttca

SEQ ID NO: 11 - DNA, 66 bp, Streptococcus pneumoniae
ggctatatgcttgcagaccgctggaggaagcacacagacggcaactggtactggttcgacaactca

SEQ ID NO: 12 - DNA, 60 bp, Streptococcus pneumoniae
ggcgaaatggctacaggctggaagaaaatcgctgataagtggtactatttcaacgaagaa

SEQ ID NO: 13 - DNA, 63 bp, Streptococcus pneumoniae
ggtgccatgaagacaggctgggtcaagtacaaggacacttggtactacttagacgctaaagaa

SEQ ID NO: 14 - DNA, 69 bp, Streptococcus pneumoniae
ggcgccatggtatcaaatgcctttatccagtcagcggacggaacaggctggtactacctcaaaccagac

SEQ ID NO: 15 - DNA, 429 bp, Streptococcus pneumoniae
ggctggcagaagaatgacactggctactggtacgtacattcagacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatggtatcaaatgcctttatccagtcagcggacggaacaggctggtactacctcaaaccagacggaacactggcagacaggccagaattcacagtagagccagatggcttgattacagtaaaataa

SEQ ID NO: 16 - DNA, 336 bp, Streptococcus pneumoniae
tacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatggtatcaaatgcctttatccagtcagcggacggaacaggctggtactacctcaaaccagac

SEQ ID NO: 17 - DNA, 1674 bp, Homo sapiens
gccaccatggtccagaggctgtgggtgagccgcctgctgcggcaccggaaagcccagctcttgctggtcaacctgctaacctttggcctggaggtgtgtttggccgcaggcatcacctatgtgccgcctctgctgctggaagtgggggtagaggagaagttcatgaccatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgtaggtcgag

SEQ ID NO: 18 - DNA, 1947 bp, Artificial Sequence
  Hybrid gene between St. pneum. C-LytA, P2 T helper epitope and human P501S.
gccaccatggcggccgcttacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaaaagttcatgtacatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgtaggtcgag

SEQ ID NO: 19 - DNA, 1662 bp, Artificial Sequence
  Codon optimised human P501S
atggtgcagcggctctgggtgagccgcctcctgcggcatcgcaaggcccagctcctgctggtgaatctgctcacattcggcctggaggtgtgcctggccgccggcatcacctacgtgccccccctcctgctggaggtgggagtcgaggagaagttcatgaccatggtgctgggcattgggcccgtcctgggcctcgtgtgcgtgcctctcctcggcagcgcttccgaccattggcgcggccggtatggccgcaggagacccttcatctgggctctgagtctcggcatcctgctgagcctgttcctgatccctcgggccggctggctggccgggctgctgtgccccgatcctcggcccctggagctggccctgctgatcctcggcgtgggcctgctggacttctgcggccaggtgtgcttcacgcccctggaggcactgctgagcgacctgttccgggaccccgaccattgccgccaggcgtacagcgtgtacgccttcatgatctccctgggaggctgcctgggctacctgctccccgccatcgattgggacaccagcgcactcgccccctatctcggaacacaggaggaatgcctgttcggattgttgacgctcatcttcctcacgtgcgtcgcggccaccctgttggtggccgaggaggccgccctggggcccaccgagccggccgagggactgagcgccccgagcctgagtccacactgctgcccttgccgggcccgcctggccttccgtaatctgggcgccctcctgcctcggctccatcagctgtgttgcagaatgcctaggacgctgcggcgcctgttcgtcgctgagttgtgctcctggatggctctcatgaccttcaccctgttttatacggacttcgtcggggagggcctgtaccagggggtgccgcgcgccgagcccgggacagaggcgcgccgccactacgacgagggagtgcgtatgggctccctgggcctcttcttgcagtgcgccatcagtctggttttctctctggtcatggacaggctggtgcagcgcttcggaacccgggcggtgtacctggcgagcgtggccgccttccccgtggctgccggcgccacctgcctctctcactcggtggccgtggtcaccgccagcgccgccctgaccgggttcaccttctctgccctgcagattctgccttacaccctggccagcctgtaccatcgcgagaaacaggtgtttctccccaagtacagaggcgacaccgggggcgcctccagcgaggacagcctcatgacctccttcctgcctggccccaagcccggcgcccctttccccaacgggcacgtgggcgccggcgggagtgggctcctgcccccccctcctgcgctgtgcggggccagcgcctgcgacgtgagcgtgcgcgtggtggtgggcgagcccaccgaggcccgcgtggtgccgggcagaggcatttgtctggacctggccatcctcgactccgccttcctcctcagccaggtggccccgtccctcttcatgggctctatcgtccagctgtctcagagcgtcaccgcttacatggtgtccgctgctggactgggcttggtggctatttatttcgccacccaggtggtgttcgacaagagcgacctggccaaatactccgcctga

SEQ ID NO: 20 - DNA, 1662 bp, Artificial Sequence
  Codon optimised human P501S
atggtgcagcggctgtgggtgtcccggctgctgcgccatagaaaggcccagttgctgctggtgaacctgctgactttcggactggaggtgtgcctggctgccgggatcacgtacgtgccccccctgctgctggaggtgggcgtggaggagaagttcatgacaatggtgctgggcatcggccccgtcctgggcctcgtgtgtgtgcccctcctcgggagtgcgtccgatcattggcggggccgctacggccgccgcagaccgttcatctgggccctgagcctggggatcctgctctctctcttcctgatcccccgggccggctggctggccggcctgctgtgtcccgacccccgccctctggagctggccctcctgatcctgggcgtgggcttgttggacttctgcggccaggtgtgtttcactcccctggaggctctgctctccgacctcttccgcgaccccgaccactgtaggcaggcttacagcgtgtacgccttcatgatcagtctggggggatgcctgggctatctgctgcccgctatcgactgggacaccagcgccctggccccctacctggggactcaggaggagtgcctgttcggcctgctcaccttgatcttcctgacgtgcgtcgccgccaccctgctggtggccgaggaggcggccctggggcccaccgagcccgccgagggcctgagcgctcccagcctgagcccccattgctgcccgtgcagggctaggctcgccttcaggaatctgggcgctttgctgccccgcctgcatcagctgtgctgtcgcatgcctcgcaccctgcgccgcctgttcgtcgctgagctctgttcctggatggccctgatgacgttcaccctcttctacaccgacttcgtgggggagggcctgtaccagggcgtgcccagggccgagcccggcaccgaggctaggcgccattacgacgagggcgtcaggatgggctctctgggcctcttcctgcagtgcgccatcagtctggtgttctctctggtgatggaccggctggtgcagcgcttcggcacccgggccgtgtacctcgcctctgtggcggctttccccgtcgccgccggcgcgacctgcctgtctcattctgtcgccgtggtgaccgccagcgccgccctgaccggcttcaccttcagtgcgctccagattctgccctacaccctggcgtctctgtaccatcgcgagaagcaggtgttcctgcccaagtaccgcggggacacagggggagcttcctctgaggacagcctgatgaccagcttcttgcccggccccaagccgggggcccctttccccaacggccatgtcggggcgggcggcagcggcctgctccctcccccccccgccctgtgcggcgctagtgcctgcgacgtgagcgtgcgggtggtggtgggggagcccaccgaggctagggtcgtgcctggccgggggatctgcctggacctggccatcctcgactccgccttcctgctctcccaggtggcgcccagcctgttcatgggcagtatcgtgcagctgagccagagcgtgaccgcctacatggtgagcgccgccggcctggggttggtggccatctactttgccacccaggtcgtgttcgacaagagcgatctcgccaagtatagcgcctga

SEQ ID NO: 21 - DNA, 1688 bp, Artificial Sequence
  Codon optimised human P501S
gacggctagcgccaccatggtgcagcggctctgggtgagccgcctcctgcggcatcgcaaggcccagctcctgctggtgaatctgctcacattcggcctggaggtgtgcctggccgccggcatcacctacgtgccccccctcctgctggaggtgggagtcgaggagaagttcatgaccatggtgctgggcattgggcccgtcctgggcctcgtgtgcgtgcctctcctcggcagcgcttccgaccattggcgcggccggtatggccgcaggagacccttcatctgggctctgagtctcggcatcctgctgagcctgttcctgatccctcgggccggctggctggccgggctgctgtgccccgatcctcggcccctggagctggccctgctgatcctcggcgtgggcctgctggacttctgcggccaggtgtgcttcacgcccctggaggcactgctgagcgacctgttccgggaccccgaccattgccgccaggcgtacagcgtgtacgccttcatgatctccctgggaggctgcctgggctacctgctccccgccatcgattgggacaccagcgcactcgccccctatctcggaacacaggaggaatgcctgttcggactgctgacgctcatcttcctcacgtgcgtcgcggccaccctgttggtggccgaggaggccgccctggggcccaccgagccggccgagggactgagcgccccgagcctgagtccacactgctgcccttgccgggcccgcctggccttccgtaatctgggcgccctcctgcctcggctccatcagctgtgttgcagaatgcctaggacgctgcggcgcctgttcgtcgctgagttgtgctcctggatggctctcatgaccttcaccctgttttatacggacttcgtcggggagggcctgtaccagggggtgccgcgcgccgagcccgggacagaggcgcgccgccactacgacgagggagtgcgtatgggctccctgggcctcttcttgcagtgcgccatcagtctggttttctctctggtcatggacaggctggtgcagcgcttcggaacccgggcggtgtacctggcgagcgtggccgccttccccgtggctgccggcgccacctgcctctctcactcggtggccgtggtcaccgccagcgccgccctgaccgggttcaccttctctgccctgcagattctgccttacaccctggccagcctgtaccatcgcgagaaacaggtgtttctccccaagtacagaggcgacaccgggggcgcctccagcgaggacagcctcatgacctccttcctgcctggccccaagcccggcgcccctttccccaacgggcacgtgggcgccggcgggagtgggctcctgcccccccctcctgcgctgtgcggggccagcgcctgcgacgtgagcgtgcgcgtggtggtgggcgagcccaccgaggcccgcgtggtgccgggcagaggcatttgtctggacctggccatcctcgactccgccttcctcctcagccaggtggccccgtccctcttcatgggctctatcgtccagctgtctcagagcgtcaccgcttacatggtgtccgctgctggactgggcttggtggctatttatttcgccacccaggtggtgttcgacaagagcgacctggccaaatactccgcctgactcgaggcag

SEQ ID NO: 22 - DNA, 1688 bp, Artificial Sequence
  Codon optimised human P501S
gacggctagcgccaccatggtgcagcggctgtgggtgtcccggctgctgcgccatagaaaggcccagttgctgctggtgaacctgctgactttcggactggaggtgtgcctggctgccgggatcacgtacgtgccccccctgctgctggaggtgggcgtggaggagaagttcatgacaatggtgctgggcatcggccccgtcctgggcctcgtgtgtgtgcccctcctcgggagtgcgtccgatcattggcggggccgctacggccgccgcagaccgttcatctgggccctgagcctgggcatcctgctctctctcttcctgatcccccgggccggctggctggccggcctgctgtgtcccgacccccgccctctggagctggccctcctgatcctgggcgtgggcctgctggacttctgcggccaggtgtgtttcactcccctggaggctctgctctccgacctcttccgcgaccccgaccactgtaggcaggcttacagcgtgtacgccttcatgatcagtctggggggatgcctgggctatctgctgcccgctatcgactgggacaccagcgccctggccccctacctggggactcaggaggagtgcctgttcggcctgctcaccttgatcttcctgacgtgcgtcgccgccaccctgctggtggccgaggaggcggccctggggcccaccgagcccgccgagggcctgagcgctcccagcctgagcccccattgctgcccgtgcagggctaggctcgccttcaggaatctgggcgctttgctgccccgcctgcatcagctgtgctgtcgcatgcctcgcaccctgcgccgcctgttcgtcgctgagctctgttcctggatggccctgatgacgttcaccctcttctacaccgacttcgtgggggagggcctgtaccagggcgtgcccagggccgagcccggcaccgaggctaggcgccattacgacgagggcgtcaggatgggctctctgggcctcttcctgcagtgcgccatcagtctggtgttctctctggtgatggaccggctggtgcagcgcttcggcacccgggccgtgtacctcgcctctgtggcggctttccccgtcgccgccggcgcgacctgcctgtctcattctgtcgccgtggtgaccgccagcgccgccctgaccggcttcaccttcagtgcgctccagattctgccctacaccctggcgtctctgtaccatcgcgagaagcaggtgttcctgcccaagtaccgcggggacacagggggagcttcctctgaggacagcctgatgaccagcttcttgcccggccccaagccgggggcccctttccccaacggccatgtcggggcgggcggcagcggcctgctccctcccccccccgccctgtgcggcgctagtgcctgcgacgtgagcgtgcgggtggtggtgggggagcccaccgaggctagggtcgtgcctggccgggggatctgcctggacctggccatcctcgactccgccttcctgctctcccaggtggcgcccagcctgttcatgggcagtatcgtgcagctgagccagagcgtgaccgcctacatggtgagcgccgccggcctggggttggtggccatctactttgccacccaggtcgtgttcgacaagagcgatctcgccaagtatagcgcctgactcgaggcag

SEQ ID NO: 23 - DNA, 435 bp, Artificial Sequence
  Hybrid gene between St. pneum. C-LytA, P2 T helper epitope and a small portion of the 5′ end of human P501S
atggcggccgcttacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaaaagttcatgtac

SEQ ID NO: 24 - DNA, 435 bp, Artificial Sequence
  Hybrid gene between St. pneum. C-LytA, P2 T helper epitope and a small portion of the 5′ end of human P501S -  codon-optimised
atggccgccgcctacgtgcatagcgacgggagctaccccaaggacaagttcgagaagatcaacgggacatggtactacttcgactcctccggctacatgctcgccgaccgctggcggaagcacaccgacggcaactggtactggttcgataactcgggagagatggccaccggctggaagaagatcgcggacaagtggtactatttcaacgaggagggcgccatgaagaccggctgggtgaagtataaggacacctggtactacctcgacgccaaggagggcgccatgcagtatatcaaggccaacagcaagttcatcggcatcaccgagggagtgatggtcagcaacgcctttatccagagcgccgacggcaccggatggtactacttgaagccggacggcaccctcgcggatcggcccgagaagttcatgtac

SEQ ID NO: 25 - DNA, 435 bp, Artificial Sequence
  Hybrid gene between St. pneum. C-LytA, P2 T helper epitope and a small portion of the 5′ end of human P501S -  codon-optimised
atggccgccgcctacgtgcacagcgacgggtcctacccaaaggacaagttcgagaagatcaacggcacgtggtactatttcgacagcagcggctacatgctcgccgatcgctggcgcaagcacaccgacgggaactggtactggttcgacaactctggcgagatggctacggggtggaagaagatcgccgacaagtggtactacttcaacgaggagggcgccatgaagaccgggtgggtgaagtacaaggacacctggtactacctggacgctaaggagggcgccatgcagtacatcaaggccaactcgaagttcatcgggatcaccgagggcgtgatggtcagtaacgctttcatccagagcgcggacggcacaggctggtattacctgaagcccgatggcaccctggcggacagacctgagaaattcatgtac

SEQ ID NO: 26 - DNA, 464 bp, Artificial Sequence
  Hybrid gene between St. pneum. C-LytA, P2 T helper epitope and a small portion of the 5′ end of human P501S -  codon-optimised
gacggctagcgccaccatggccgccgcctacgtgcatagcgacgggagctaccccaaggacaagttcgagaagatcaacgggacatggtactacttcgactcctccggctacatgctcgccgaccgctggcggaagcacaccgacggcaactggtactggttcgataactcgggagagatggccaccggctggaagaagatcgcggacaagtggtactatttcaacgaggagggcgccatgaagaccggctgggtgaagtataaggacacctggtactacctcgacgccaaggagggcgccatgcagtatatcaaggccaacagcaagttcatcggcatcaccgagggagtgatggtcagcaacgcctttatccagagcgccgacggcaccggatggtactacttgaagccggacggcaccctcgcggatcggcccgagaagttcatgtactgactcgaggcag

SEQ ID NO: 27 - protein, 652 aa, Artificial Sequence
  Hybrid protein between St. pneum. C-LytA, P2 T helper epitope and amino acids 51-553 of human P501S
MAAAYVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEKFMYMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSAGGHHHHHH

SEQ ID NO: 28 - DNA, 1959 bp, Artificial Sequence
  DNA encoding the Hybrid protein between St. pneum. C-LytA, P2 T helper epitope and amino acids 51-553of human P501S
atggcggccgcttacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaaaagttcatgtacatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgggtggacaccatcaccatcaccattaa

SEQ ID NO: 29 - protein, 507 aa, Artificial Sequence
  Human P501S (amino acids 55-553) fused to 6 histidine residues
MVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSAGGHHHHHH

SEQ ID NO: 30 - DNA, 1524 bp, Artificial Sequence
  DNA encoding Human P501S (amino acids 55-553) fused to 6 histidine residues
atggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgggtggacaccatcaccatcaccattaa

SEQ ID NO: 31 - protein, 685 aa, Artificial Sequence
  Human P501S (amino acids 1-34 fused to 55-553) fused to 6 histidine residues
MAAVQRLWVSRLLRHRKAQLLLVNLLTFGLEVCLAAAYVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEKFMYMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSAGGHHHHHH

SEQ ID NO: 32 - DNA, 2058 bp, Artificial Sequence
  DNA encoding Human P501S (amino acids 1-34 fusedto 55-553) fused to 6 histidine residues
atggcggccgtgcagaggctatgggtatcgagactgctaagacaccgcaaagctcagttgttgttggttaacttgttgaccttcgggctggaagtctgtttggcggccgcttacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaaaagttcatgtacatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgggtggacaccatcaccatcaccattaa

SEQ ID NO: 33 - protein, 671 aa, Artificial Sequence
  St. pneum. C-LytA portion fused to P2 T helper epitope fused to Human P501S (amino acids 55-553) fused to 6 histidine residues downstream of yeast alphaprepro signal sequence
MAARFPSIFTAVLFAASSALAAAYVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEKFMYMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSAGGHHHHHH

SEQ ID NO: 34 - DNA, 2477 bp, Artificial Sequence
  DNA encoding St. pneum. C-LytA portion fused to P2 T helper epitope fused to Human P501S (amino acids 55-553) fused to 6 histidine residues downstream of yeast alphaprepro signal sequence
tacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaaatggcggccagatttccttcaatttttactgcagttttattcgcagcatcctccgcattagcggccgcttacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaagctggtattacttacgttccaccattgttgttggaagttggtgttgaagaaaagttcatgtacatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgcttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgggtggacaccatcaccatcaccattaa

SEQ ID NO: 35 - protein, 595 aa, Artificial Sequence
  Human P501S (amino acids 55-553) fused to 6 histidine residues downstream of yeast alphaprepro signal sequence
MSFLNFTAVLFAASSALAAPVNTTTEDETAQIPAEAVIGYSDLEGDFDVAVLPFSNSTNNGLLFINTTIASIAAKEEGVSLEKREAEAMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSAGGHHHHHH

SEQ ID NO: 36 - DNA, 1788 bp, Artificial Sequence
  DNA encoding Human P501S (amino acids 55-553) fused to 6 histidine residues downstream of yeast alphaprepro signal sequence
atgagtttcctcaattttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagccatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgggtggacaccatcaccatcaccattaa

SEQ ID NO: 37 - DNA, 1955 bp, Artificial Sequence
  DNA encoding codon-optimised Human P501S (amino acids 51-553) fused to St.pneum. C-LytA P2 helper epitope C-Lyta
gcggccgcgccaccatggccgccgcctacgtgcatagcgacgggagctaccccaaggacaagttcgagaagatcaacgggacatggtactacttcgactcctccggctacatgctcgccgaccgctggcggaagcacaccgacggcaactggtactggttcgataactcgggagagatggccaccggctggaagaagatcgcggacaagtggtactatttcaacgaggagggcgccatgaagaccggctgggtgaagtataaggacacctggtactacctcgacgccaaggagggcgccatgcagtatatcaaggccaacagcaagttcatcggcatcaccgagggagtgatggtcagcaacgcctttatccagagcgccgacggcaccggatggtactacttgaagccggacggcaccctcgcggatcggcccgagaagttcatgtacatggtgctgggcatcggccccgtcctgggcctcgtgtgtgtgcccctcctcgggagtgcgtccgatcattggcggggccgctacggccgccgcagaccgttcatctgggccctgagcctgggcatcctgctctctctcttcctgatcccccgggccggctggctggccggcctgctgtgtcccgacccccgccctctggagctggccctcctgatcctgggcgtgggcctgctggacttctgcggccaggtgtgtttcactcccctggaggctctgctctccgacctcttccgcgaccccgaccactgtaggcaggcttacagcgtgtacgccttcatgatcagtctggggggatgcctgggctatctgctgcccgctatcgactgggacaccagcgccctggccccctacctggggactcaggaggagtgcctgttcggcctgctcaccttgatcttcctgacgtgcgtcgccgccaccctgctggtggccgaggaggcggccctggggcccaccgagcccgccgagggcctgagcgctcccagcctgagcccccattgctgcccgtgcagggctaggctcgccttcaggaatctgggcgctttgctgccccgcctgcatcagctgtgctgtcgcatgcctcgcaccctgcgccgcctgttcgtcgctgagctctgttcctggatggccctgatgacgttcaccctcttctacaccgacttcgtgggggagggcctgtaccagggcgtgcccagggccgagcccggcaccgaggctaggcgccattacgacgagggcgtcaggatgggctctctgggcctcttcctgcagtgcgccatcagtctggtgttctctctggtgatggaccggctggtgcagcgcttcggcacccgggccgtgtacctcgcctctgtggcggctttccccgtcgccgccggcgcgacctgcctgtctcattctgtcgccgtggtgaccgccagcgccgccctgaccggcttcaccttcagtgcgctccagattctgccctacaccctggcgtctctgtaccatcgcgagaagcaggtgttcctgcccaagtaccgcggggacacagggggagcttcctctgaggacagcctgatgaccagcttcttgcccggccccaagccgggggcccctttccccaacggccatgtcggggcgggcggcagcggcctgctccctcccccccccgccctgtgcggcgctagtgcctgcgacgtgagcgtgcgggtggtggtgggggagcccaccgaggctagggtcgtgcctggccgggggatctgcctggacctggccatcctcgactccgccttcctgctctcccaggtggcgcccagcctgttcatgggcagtatcgtgcagctgagccagagcgtgaccgcctacatggtgagcgccgccggcctggggttggtggccatctactttgccacccaggtcgtgttcgacaagagcgatctcgccaagtatagcgcctgaggatcc

SEQ ID NO: 38 - DNA, 2045 bp, Artificial Sequence
  DNA encoding codon-optimised Human P501S (amino acids 1-553) fused to St.pneum. C-LytA P2 helper epitope C-Lyta
gcggccgcgccaccatggccgccgcctacgtgcatagcgacgggagctaccccaaggacaagttcgagaagatcaacgggacatggtactacttcgactcctccggctacatgctcgccgaccgctggcggaagcacaccgacggcaactggtactggttcgataactcgggagagatggccaccggctggaagaagatcgcggacaagtggtactatttcaacgaggagggcgccatgaagaccggctgggtgaagtataaggacacctggtactacctcgacgccaaggagggcgccatgcagtatatcaaggccaacagcaagttcatcggcatcaccgagggagtgatggtcagcaacgcctttatccagagcgccgacggcaccggatggtactacttgaagccggacggcaccctcgcggatcggcccgagatggtgcagcggctgtgggtgtcccggctgctgcgccatagaaaggcccagttgctgctggtgaacctgctgactttcggactggaggtgtgcctggctgccgtggtgctgggcatcggccccgtcctgggcctcgtgtgtgtgcccctcctcgggagtgcgtccgatcattggcggggccgctacggccgccgcagaccgttcatctgggccctgagcctgggcatcctgctctctctcttcctgatcccccgggccggctggctggccggcctgctgtgtcccgacccccgccctctggagctggccctcctgatcctgggcgtgggcctgctggacttctgcggccaggtgtgtttcactcccctggaggctctgctctccgacctcttccgcgaccccgaccactgtaggcaggcttacagcgtgtacgccttcatgatcagtctggggggatgcctgggctatctgctgcccgctatcgactgggacaccagcgccctggccccctacctggggactcaggaggagtgcctgttcggcctgctcaccttgatcttcctgacgtgcgtcgccgccaccctgctggtggccgaggaggcggccctggggcccaccgagcccgccgagggcctgagcgctcccagcctgagcccccattgctgcccgtgcagggctaggctcgccttcaggaatctgggcgctttgctgccccgcctgcatcagctgtgctgtcgcatgcctcgcaccctgcgccgcctgttcgtcgctgagctctgttcctggatggccctgatgacgttcaccctcttctacaccgacttcgtgggggagggcctgtaccagggcgtgcccagggccgagcccggcaccgaggctaggcgccattacgacgagggcgtcaggatgggctctctgggcctcttcctgcagtgcgccatcagtctggtgttctctctggtgatggaccggctggtgcagcgcttcggcacccgggccgtgtacctcgcctctgtggcggctttccccgtcgccgccggcgcgacctgcctgtctcattctgtcgccgtggtgaccgccagcgccgccctgaccggcttcaccttcagtgcgctccagattctgccctacaccctggcgtctctgtaccatcgcgagaagcaggtgttcctgcccaagtaccgcggggacacagggggagcttcctctgaggacagcctgatgaccagcttcttgcccggccccaagccgggggcccctttccccaacggccatgtcggggcgggcggcagcggcctgctccctcccccccccgccctgtgcggcgctagtgcctgcgacgtgagcgtgcgggtggtggtgggggagcccaccgaggctagggtcgtgcctggccgggggatctgcctggacctggccatcctcgactccgccttcctgctctcccaggtggcgcccagcctgttcatgggcagtatcgtgcagctgagccagagcgtgaccgcctacatggtgagcgccgccggcctggggttggtggccatctactttgccacccaggtcgtgttcgacaagagcgatctcgccaagtatagcgcctgaggatcc

SEQ ID NO: 39 - DNA, 2105 bp, Artificial Sequence
  DNA encoding St.pneum. C-LytA P2 helper epitope C-Lyta fused to Human P501S (amino acids 51-553) fused to Human P501S (amino acids 1-50)  - Codon-optimised
gcggccgcgccaccatggccgccgcctacgtgcatagcgacgggagctaccccaaggacaagttcgagaagatcaacgggacatggtactacttcgactcctccggctacatgctcgccgaccgctggcggaagcacaccgacggcaactggtactggttcgataactcgggagagatggccaccggctggaagaagatcgcggacaagtggtactatttcaacgaggagggcgccatgaagaccggctgggtgaagtataaggacacctggtactacctcgacgccaaggagggcgccatgcagtatatcaaggccaacagcaagttcatcggcatcaccgagggagtgatggtcagcaacgcctttatccagagcgccgacggcaccggatggtactacttgaagccggacggcaccctcgcggatcggcccgagaagttcatgtacatggtgctgggcatcggccccgtcctgggcctcgtgtgtgtgcccctcctcgggagtgcgtccgatcattggcggggccgctacggccgccgcagaccgttcatctgggccctgagcctgggcatcctgctctctctcttcctgatcccccgggccggctggctggccggcctgctgtgtcccgacccccgccctctggagctggccctcctgatcctgggcgtgggcctgctggacttctgcggccaggtgtgtttcactcccctggaggctctgctctccgacctcttccgcgaccccgaccactgtaggcaggcttacagcgtgtacgccttcatgatcagtctggggggatgcctgggctatctgctgcccgctatcgactgggacaccagcgccctggccccctacctggggactcaggaggagtgcctgttcggcctgctcaccttgatcttcctgacgtgcgtcgccgccaccctgctggtggccgaggaggcggccctggggcccaccgagcccgccgagggcctgagcgctcccagcctgagcccccattgctgcccgtgcagggctaggctcgccttcaggaatctgggcgctttgctgccccgcctgcatcagctgtgctgtcgcatgcctcgcaccctgcgccgcctgttcgtcgctgagctctgttcctggatggccctgatgacgttcaccctcttctacaccgacttcgtgggggagggcctgtaccagggcgtgcccagggccgagcccggcaccgaggctaggcgccattacgacgagggcgtcaggatgggctctctgggcctcttcctgcagtgcgccatcagtctggtgttctctctggtgatggaccggctggtgcagcgcttcggcacccgggccgtgtacctcgcctctgtggcggctttccccgtcgccgccggcgcgacctgcctgtctcattctgtcgccgtggtgaccgccagcgccgccctgaccggcttcaccttcagtgcgctccagattctgccctacaccctggcgtctctgtaccatcgcgagaagcaggtgttcctgcccaagtaccgcggggacacagggggagcttcctctgaggacagcctgatgaccagcttcttgcccggccccaagccgggggcccctttccccaacggccatgtcggggcgggcggcagcggcctgctccctcccccccccgccctgtgcggcgctagtgcctgcgacgtgagcgtgcgggtggtggtgggggagcccaccgaggctagggtcgtgcctggccgggggatctgcctggacctggccatcctcgactccgccttcctgctctcccaggtggcgcccagcctgttcatgggcagtatcgtgcagctgagccagagcgtgaccgcctacatggtgagcgccgccggcctggggttggtggccatctactttgccacccaggtcgtgttcgacaagagcgatctcgccaagtatagcgccatggtgcagcggctgtgggtgtcccggctgctgcgccatagaaaggcccagttgctgctggtgaacctgctgactttcggactggaggtgtgcctggctgccgggatcacgtacgtgccccccctgctgctggaggtgggcgtggaggagtgaggatcc

SEQ ID NO: 40 - DNA, 2105 bp, Artificial Sequence
  DNA encoding Human P501S (amino acids 1-50) fusedto St.pneum. C-LytA P2 helper epitope C-Lyta fused to Human P501S (amino acids 51-553)  -Codon-optimised
gcggccgcgccaccatggtgcagcggctgtgggtgtcccggctgctgcgccatagaaaggcccagttgctgctggtgaacctgctgactttcggactggaggtgtgcctggctgccgggatcacgtacgtgccccccctgctgctggaggtgggcgtggaggagatggccgccgcctacgtgcatagcgacgggagctaccccaaggacaagttcgagaagatcaacgggacatggtactacttcgactcctccggctacatgctcgccgaccgctggcggaagcacaccgacggcaactggtactggttcgataactcgggagagatggccaccggctggaagaagatcgcggacaagtggtactatttcaacgaggagggcgccatgaagaccggctgggtgaagtataaggacacctggtactacctcgacgccaaggagggcgccatgcagtatatcaaggccaacagcaagttcatcggcatcaccgagggagtgatggtcagcaacgcctttatccagagcgccgacggcaccggatggtactacttgaagccggacggcaccctcgcggatcggcccgagaagttcatgtacatggtgctgggcatcggccccgtcctgggcctcgtgtgtgtgcccctcctcgggagtgcgtccgatcattggcggggccgctacggccgccgcagaccgttcatctgggccctgagcctgggcatcctgctctctctcttcctgatcccccgggccggctggctggccggcctgctgtgtcccgacccccgccctctggagctggccctcctgatcctgggcgtgggcctgctggacttctgcggccaggtgtgtttcactcccctggaggctctgctctccgacctcttccgcgaccccgaccactgtaggcaggcttacagcgtgtacgccttcatgatcagtctggggggatgcctgggctatctgctgcccgctatcgactgggacaccagcgccctggccccctacctggggactcaggaggagtgcctgttcggcctgctcaccttgatcttcctgacgtgcgtcgccgccaccctgctggtggccgaggaggcggccctggggcccaccgagcccgccgagggcctgagcgctcccagcctgagcccccattgctgcccgtgcagggctaggctcgccttcaggaatctgggcgctttgctgccccgcctgcatcagctgtgctgtcgcatgcctcgcaccctgcgccgcctgttcgtcgctgagctctgttcctggatggccctgatgacgttcaccctcttctacaccgacttcgtgggggagggcctgtaccagggcgtgcccagggccgagcccggcaccgaggctaggcgccattacgacgagggcgtcaggatgggctctctgggcctcttcctgcagtgcgccatcagtctggtgttctctctggtgatggaccggctggtgcagcgcttcggcacccgggccgtgtacctcgcctctgtggcggctttccccgtcgccgccggcgcgacctgcctgtctcattctgtcgccgtggtgaccgccagcgccgccctgaccggcttcaccttcagtgcgctccagattctgccctacaccctggcgtctctgtaccatcgcgagaagcaggtgttcctgcccaagtaccgcggggacacagggggagcttcctctgaggacagcctgatgaccagcttcttgcccggccccaagccgggggcccctttccccaacggccatgtcggggcgggcggcagcggcctgctccctcccccccccgccctgtgcggcgctagtgcctgcgacgtgagcgtgcgggtggtggtgggggagcccaccgaggctagggtcgtgcctggccgggggatctgcctggacctggccatcctcgactccgccttcctgctctcccaggtggcgcccagcctgttcatgggcagtatcgtgcagctgagccagagcgtgaccgcctacatggtgagcgccgccggcctggggttggtggccatctactttgccacccaggtcgtgttcgacaagagcgatctcgccaagtatagcgcctgaggatcc

SEQ ID NO: 41 - protein, 652 aa, Artificial Sequence; identical to SEQ ID NO: 27
  St.pneum. C-LytA P2 helper epitope C-Lyta fused to Human P501S

SEQ ID NO: 42 - DNA, 1959 bp, Artificial Sequence; identical to SEQ ID NO: 28
  DNA encoding St.pneum. C-LytA P2 helper epitope C-Lyta fused to Human P501S (plus his tag)

SEQ ID NO: 43 - protein, 553 aa, Homo sapiens
MVQRLWVSRLLRHRKAQLLLVNLLTFGLEVCLAAGITYVPPLLLEVGVEEKFMTMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 44 - protein, 644 aa, Artificial Sequence
  St.pneum. C-LytA P2 helper epitope C-Lyta fused to Human P501S
MAAAYVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEKFMYMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 45 - protein, 644 aa, Artificial Sequence; identical to SEQ ID NO: 44
  Codon-optimised hybrid protein between St. pneum. C-LytA P2 helper epitope C-Lyta fused to Human P501S amino acids 51-553)

SEQ ID NO: 46 - protein, 694 aa, Artificial Sequence [sequence omitted]

SEQ ID NO: 47 - protein, 694 aa, Artificial Sequence
  St.pneum. C-LytA P2 helper epitope C-Lyta fused to Human P501S (amino acids 51-553) fused to Human P501S (amino acids 1-50) - codon-optimised
MAAAYVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEKFMYMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSAMVQRLWVSRLLRHRKAQLLLVNLLTFGLEVCLAAGITYVPPLLLEVGVEE

SEQ ID NO: 48 - protein, 694 aa, Artificial Sequence
  Human P501S (amino acids 1-50) fused to St. pneum. C-LytA P2 helper epitope C-Lyta fused to Human P501S (amino acids 51-553) - codon optimised
MVQRLWVSRLLRHRKAQLLLVNLLTFGLEVCLAAGITYVPPLLLEVGVEEMAAAYVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEKFMYMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 49 - DNA, 1971 bp, Artificial Sequence
  DNA encoding Human MUC-1 fused to St.pneum. C-LytA P2 helper epitope C-Lyta
atgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccgcccccgggctccaccgcccccccagcccacggtgtcacctcggccccggacaccaggccgcccccgggctccaccgcgcccgcagcccacggtgtcacctcggccccggacaccaggccggccccgggctccaccgcccccccagcccatggtgtcacctcggccccggacaacaggcccgccttggcgtccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgatggcggccgcttacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaatga

SEQ ID NO: 50 - protein, 656 aa, Artificial Sequence
  Human MUC-1 fused to St.pneum. C-LytA P2 helper epitope C-Lyta
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPPPGSTAPPAHGVTSAPDTRPPPGSTAPAAHGVTSAPDTRPAPGSTAPPAHGVTSAPDNRPALASTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANLMAAAYVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPE

SEQ ID NO: 51 - DNA, 2037 bp, Artificial Sequence
  DNA encoding St.pneum. C-LytA P2 helper epitope C-Lyta  fused to Human MUC-1
atgggatggagctgtatcatcctcttcttggtagcaacagctacaggtgtccactcccaggtccaaatggcggccgcttacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaaatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccgcccccgggctccaccgcccccccagcccacggtgtcacctcggccccggacaccaggccgcccccgggctccaccgcgcccgcagcccacggtgtcacctcggccccggacaccaggccggccccgggctccaccgcccccccagcccatggtgtcacctcggccccggacaacaggcccgccttggcgtccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtag

SEQ ID NO: 52 - protein, 678 aa, Artificial Sequence [sequence omitted]

(19) United States
(12) Patent Application Publication
(10) Pub. No.: US 2006/0147477 A1
(43) Pub. Date: Jul. 6, 2006

(54) Immunogenic compositions

(75) Inventors: Teresa Elisa Virginia Cabezon Siliva, Rixensart (BE); Jonathan H Ellis, Hertfordshire (GB); Catherine Marie Ghislaine, Rixensart (BE); Paul A Hamblin, Hertfordshire (GB); Remi M Palmantier, Rixensart (BE); Vinals Y. De Bassols, Rixensart (BE)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Glaxo Group Limited

(21) Appl. No.: 10/517,420

(22) PCT Filed: Jun. 6, 2003
(86) PCT No.: PCT/EP03/06096

§ 371 Date: Oct. 18, 2005

(30) Foreign Application Priority Data
Jun. 11, 2002 (GB) 0213365.0
Jan. 15, 2003 (GB) 0300914.9

(51) Int. Cl. A61K 39/00; A61K 48/00; C07K 14/82; C07H 21/04; C12P 21/06

(52) U.S. Cl. 424/277.1; 435/69.1; 435/320.1; 435/325; 514/44; 530/350; 536/23.5

(57) ABSTRACT

The present invention relates fusion partners which act as immunological fusion partners, as expression enhancers, and preferably to fusion partners having both functions. In particular the fusion partners contain a so-called choline binding domain, for example fusions comprising LytA from Streptococcus pneumoniae, or the pneumococcal phage CP1 lysozyme (CPL1) wherein the choline binding domain is modified to include a heterologous T-helper epitope, and are fused to antigens, particularly poorly immunogenic antigens such as self-antigens, eg tumour specific or tissue specific antigens. The invention also relates to fusion proteins containing them, to their manufacture, to their use in immunogenic compositions and vaccines and to their use in medicines.

The present invention relates to fusion partners which act as immunological fusion partners, as expression enhancers, and preferably to fusion partners having both functions. The invention also relates to fusion proteins containing them, to their manufacture, to their use in vaccines and to their use in medicines. In particular fusion partners are provided that contain a so-called choline binding domain, for example fusions comprising LytA from Streptococcus pneumoniae, or the pneumococcal phage CP1 lysozyme (CPL1) wherein the choline binding domain is modified to include a heterologous T-helper epitope. Such fusion partners are shown to improve the expression level of the heterologous protein attached thereto and also find particular utility when fused to poorly immunogenic proteins or peptides that are otherwise useful as vaccine antigens. More particularly, such fusion partners are useful in constructs comprising self-antigens, eg tumour specific or tissue specific antigens.

### BACKGROUND TO THE INVENTION

Streptococcus pneumoniae synthesises an N acetyl-L-alanine amidase, LytA, an autolysin that specifically degrades the peptidoglycan backbone of the cell wall eventually leading to cell lysis. Its polypeptide chain has two domains. The N-terminal domain is responsible for the catalytic activity, whereas the C-terminal domain of LytA is responsible for the affinity to choline and anchorage to the cell wall. This C-terminal domain is known to bind to choline and choline analogues, and will also bind to tertiary amines such as DEAE (diethyl amino ethyl) commonly used in chromatography.

LytA is a 318 amino acid protein, and the C-terminal part comprises a tandem of six imperfect repeats of 20 or 21 amino acids and a short COOH-terminal tail. The repeats are located at the following positions:

R1: 177-191

R2: 192-212

R3: 213-234

R4: 235-254

R5: 255-275

R6: 276-298

These repeats are predicted to be in a beta-turn conformation. The C-terminus is responsible for binding choline. Likewise the C-terminus of CPL1 is responsible for binding affinity and the aromatic residues in the repeat contribute to such binding. These proteins have been used as affinity tags to allow for rapid purification (Sanchez Puelles, Eur J Biochem. 1992, 203, 153-9).

Other proteins with a choline-binding domain have also been studied in Streptococcus pneumoniae.

One of them PspA (or Pneumococcal Surface Protein A), is a virulence factor (Yother J and Briles (1992) J Bacteriol 174(2) p 601). This protein is antigenic and immunogenic. It has a C-terminal domain consisting of 10 repeats of 20 amino acids, homologous with repeats of LytA.

CbpA (or Choline-Binding Protein A) is involved in the adherence of the pneumococcus to human cells (Rosenow et al (1997) Mol Microbiol 25 (5) p 819). It shows 10 repeats of 20 amino acids in the C-terminal domain which are almost identical to those of PspA.

LytB and LytC have a different modular organisation from the above-mentioned proteins as their choline-binding domain, made up of 15 repeats and 11 repeats respectively, is situated at the N-terminal end, not at the C-terminal end (Garcia P Mol Microbiol (1999) 31 (4) p1275 and Garcia P et al (1999) Mol Microbiol 33(1) p128). Sequence comparison shows LytB to have glucosamidase activity. LytC shows in vitro a lysozyme-type activity. Additionally, three genes called PepA, PepB and PepC were cloned in 1995. Although their function is unknown, these genes also have a variable number of repeats homologous to those of LytA.

In their infection cycle, phages synthesise murein hydrolases facilitating their passage into the bacterium. These hydrolases have a choline-binding domain.

The muramidase CPL1 of the phage Cp-1 has been well studied. It shows 6 repeats of 20 amino acids at the C-terminus involved in the specific recognition of choline (Garica J. L. J. Virol 61 (8) p2573-80; (1987) and Garcia E Prol Natl Acad Sci (1988) p914). A comparison of the LytA and CPL1 repeats enables an initial consensus of those repeats to be made.

The murein hydrolases of phages Dp-1 (Garcia P et al (1983) J Gen Microbiol 129 (2) p489, Cpl-9 (Garcia P et al (1989) Biochem Biophys Res Commun 158(1) p 251, HB-3 Romero et al 1990 J Bacteriol 172 (9) p 5064-5070) and EJ-1 Diaz (1992) J Bacteriol 174 (17) p 5516), also show the characteristics of choline-binding domains.

This property is also shared by the lysozyme encoded by CP-1 a pneumococal phage. WO 99/10375 describes inter alia, human papilloma virus proteins E6, or E7 linked to a His tag and the C-terminal portion of LytA (herein (C-LytA) and the purification of the proteins by differential affinity chromatography.

WO 99/40188 describes inter alia fusion proteins comprising MAGE antigens with a His tails and a C-LytA portion at the N-terminus of the molecule.

It has now been surprisingly found that fusion partners according to the present invention, when fused to a heterologous protein were capable of enhancing the immunogenicity of the heterologous proteins attached thereto. It has also been found that the expression level of the heterologous proteins attached thereto can be enhanced. The present invention accordingly provides in a preferred embodiment an improved immunological fusion partner which can also act as an expression enhancer.

### SUMMARY OF THE INVENTION

Accordingly the present invention comprises a fusion partner molecule comprising a choline binding domain or a fragment thereof or an analogue thereof, and a heterologous promiscuous T helper epitope, preferably a promiscuous MHC Class II T-epitope. Said fusion partner shows a capability of acting as both an immunological fusion partner, or as an expression enhancer and preferably as both an immunological partner and expression enhancer. A promiscuous T-helper epitope is an epitope that binds to more than one MHC Class II allele, preferably more than 3 MHC Class II alleles. In particular such epitopes are capable of eliciting helper T cell response in large numbers of individuals expressing diverse MHC haplotypes. Optionally, the fusion protein may retain its capability to bind to choline.

In a preferred embodiment the choline binding moiety is derived from the C terminus of LytA. Preferably the C-LytA or derivatives comprises at least four repeats of any of the repeats R1 to R6 set forth in FIG. 1 (SEQ ID NO:1 to 6). In a most preferred embodiment, the C-LytA extends from amino acid 177-298 which contains a portion of the first repeat and the complete five others.

In a further aspect of the invention, there is provided a fusion partner as herein defined further comprising a heterologous protein. The heterologous protein may be either chemically conjugated or fused to the fusion partner. Preferably the heterologous protein is a tumour-associated antigen or immunogenic fragment thereof.

In a further aspect of the invention there is provided a nucleic acid sequence encoding the proteins as herein defined. There is also provided an expression vector comprising said nucleic acid, and a host transformed with said nucleic acid or vector.

In a further aspect of the invention there is provided an immunogenic composition comprising a protein or a nucleic acid sequence as herein described, and a pharmaceutically acceptable excipient, diluent or carrier. Preferably the immunogenic composition further comprises a Th-1 inducing adjuvant.

In yet a further embodiment, the invention provides the immunogenic composition or protein and nucleic acids for use in medicine. In particular, there is provided a protein or a nucleic acid of the invention, in the manufacture of a medicament for eliciting an immune response in a patient, or for use in the treatment or prophylaxis of infectious diseases or cancer diseases.

The invention further provides for methods of treating a patient suffering from an infectious disease or a cancer disease, particularly carcinoma of the breast, lung (particularly non-small cell lung carcinoma), colorectal, ovarian, prostate, gastric and other GI (gastrointestinal) by the administration of a safe and effective amount of a composition or nucleic acid as herein described.

In yet a further embodiment the invention provides a method of producing an immunogenic composition as herein described by admixing a nucleic acid or protein of the invention with a pharmaceutically acceptable excipient, diluent or carrier.

### DETAILED DESCRIPTION OF THE INVENTION

As described therein, in one embodiment of the present invention the modified choline binding domain (fusion partner) has a capability of acting as an expression enhancer with the resulting fusion protein will be expressed at a higher yield in a host cell as compared to the unfused protein, preferably at a yield greater than about 100% (2-fold higher) or 150% or more, as measured by SDS-PAGE followed by Coomassie blue staining or silver staining, optionally followed by gel scanning. The modified choline binding domain according to the invention has also the capability of acting as an immunological partner with the resulting fusion protein with a heterologous protein will be more immunogenic in a host as compared to the unfused heterologous protein.

In another embodiment of the present invention, the modified choline binding domain has the capability to act as an immunological fusion partner, allowing an enhanced immune response to be obtained with the fusion protein as compared to the heterologous protein alone.

In a preferred embodiment, the modified choline binding domain has a dual function, having the capability to act as both an immunological fusion partner and as an expression enhancer.

In a preferred embodiment the choline binding moiety is derived from the C terminus of LytA. Preferably the C-LytA or derivatives comprises at least two repeats, preferably at least four repeats. In this context, C-LytA derivatives refer to a variant of C-LytA according to the present invention, that is to say variants which have retained both the capability of acting as an immunological partner and an expression enhancer. Preferred variants include, for example, peptides comprising an amino acid sequence having at least 85% identity, preferably at least 90% identity, more preferably at least 95% identity, most preferably at least 97-99% identity, to any of the repeats R1 to R6 set forth in FIG. 1 (SEQ ID NO:1 to 6), or a peptide comprising an amino acid sequence having at least 15, 20, 30, 40, 50 or 100 contiguous amino acids from the amino acid sequence set forth in FIG. 1 (SEQ ID NO:1 to 8).

Accordingly, in one aspect of the invention there is provided a fusion partner protein comprising a modified choline binding domain and a heterologous promiscuous T helper epitope, wherein the choline binding domain is selected from the group comprising:
a) the C-terminal domain of LytA as set forth in SEQ ID NO:7;
b) the sequence of SEQ ID NO:8;
c) a peptide sequence comprising an amino acid sequence having at least 85% identity, preferably at least 90% identity, more preferably at least 95% identity, most preferably at least 97-99% identity, to any of SEQ ID NO:1 to 6;
d) a peptide sequence comprising an amino acid sequence having at least 15, 20, 30, 40, 50 or 100 contiguous amino acids from the amino acid sequence of SEQ ID NO:7 or SEQ ID NO:8.

In a most preferred embodiment, the C-LytA extends from amino acid 177-298 which contains a portion of the first repeat and the complete five others, as set forth in FIG. 1.

The second component of the fusion partner, the heterologous T-cell epitope is preferably selected from the group of epitopes that will bind to a number of individuals expressing more than one MHC II molecules in humans. For example, epitopes that are specifically contemplated are P2 and P30 epitopes from tetanus toxoid, Panina-Bordignon Eur. J. Immunol 19 (12), 2237 (1989). In a preferred embodiment the heterologous T-cell epitope is P2 or P30 from Tetanus toxin.

The P2 epitope has the sequence QYIKANSKFIGITE and corresponds to amino acids 830-843 of the Tetanus toxin. The P30 epitope (residues 947-967 of Tetanus Toxin) has the sequence FNNFTVSFWLRVPKVSASHLE. The FNNFTV sequence may optionally be deleted. Other universal T epitopes can be derived from the circumsporozoite protein from Plasmodium falciparum—in particular the region 378-398 having the sequence DIEKKIAKMEKASSVFNWNS (Alexander J, (1994) Immunity 1 (9), p 751-761). Another epitope is derived from Measles virus fusion protein at residue 288-302 having the sequence LSEIKGVIVHRLEGV (Partidos C D, 1990, J. Gen. Virol 71(9) 2099-2105). Yet another epitope is derived from hepatitis B virus surface antigen, in particular amino acids, having the sequence FFLLTRILTIPQSLD. Another set of epitopes is derived from diphteria toxin. Four of these peptides (amino acids 271-290, 321-340, 331-350, 351-370) map within the T domain of fragment B of the toxin, and the remaining 2 map in the R domain (411-430, 431-450):

PVFAGANYAAWAVNVAQVI

VHHNTEEIVAQSIALSSLMV

QSIALSSLMVAQAIPLVGEL

VDIGFMYNFVESII NLFQV

QGESGHDIKITAENTPLPIA

GVLLPTIPGKLDVNKSKTHI

(Raju R., Navaneetham D., Okita D., Diethelm-Okita B., McCormick D., Conti-Fine B. M. (1995) Eur. J. Immunol. 25: 3207-14.)

The heterologous T-epitope is preferably fused to C-LytA containing at least 4 repeats, preferably repeat 2-5 inclusive. One or more subsequent repeats may optionally be fused to the C-terminus of the T-epitope. Alternatively, the heterologous T-epitope is preferably inserted between two consecutive repeats of C-LytA containing a total of at least 4 repeats, or inserted into one of the repeats of C-LytA containing a total of at least 4 repeats. More preferably, the C-LytA contains 6 repeats and the heterologous epitope is inserted within and at the beginning of the sixth repeat of C-LytA.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant.

Thus a self-protein or other poorly immunogenic protein may be fused to either the N or C terminal end of the resulting fusion partner. Alternatively the self protein or poorly immunogenic protein may be inserted into the fusion partner. In an optional embodiment a histidine tag or at least four, preferably more than 6 histidine residues, may be fused to the alternative end of the poorly immunogenic protein. This would allow for the protein to be purified by affinity chromatography steps, as a histidine tail, typically comprising at least four, preferably six or more residues binds to metal ions and therefore is suitable for metal immobilised metal ion affinity chromatography (IMAC).

Typical constructs would therefore comprise:
Poorly-immunogenic protein—C-LytA repeats₁₋₄-P₂ epitope (inserted in or replacing C-LytA repeat₅)-C-LytA repeat₆
C-LytA repeats₁₋₄-P₂ epitope (inserted in or replacing C-LytA repeat₅)—C-LytA repeat₆—Poorly immunogenic protein
Poorly immunogenic protein—C-LytA repeat₂₋₅-P₂ epitope (inserted into C-LytA repeat₆₎
C-LytA₂₋₅-P₂epitope (inserted into C-LytA repeats)—Poorly immunogenic protein.
Poorly immunogenic protein C-LytA repeats₁₋₅-P₂ epitope-inserted in C-LytA repeat
C-LytA repeats₁₋₅-P₂ epitope-inserted in C-LytA repeat₆—Poorly immunogenic protein
Poorly immunogenic protein-P₂ epitope inserted into C-LytA repeat₁-C-LytA repeats₂₋₅
P₂ epitope inserted into C-LytA repeat₁-C-LytA repeats₂₋₅—Poorly immunogenic protein
Poorly immunogenic protein-P₂ epitope inserted into C-LytA repeat₁-C-LytA repeats₂₋₆
P₂ epitope inserted into C-LytA repeat₁-C-LytA repeats₂₋₈-Poorly immunogenic protein
Poorly immunogenic protein-C-LytA repeat₁-P₂ epitope inserted into C-LytA repeat₂-C-LytA repeats₃₋₆
C-LytA repeat₁-P₂ epitope inserted into C-LytA repeat₂-C-LytA repeats₃₋₆-Poorly immunogenic protein;
   where “inserted into” means at any place into the repeat for example between residue 1 and 2, or between 2 and 3, etc.

The promiscuous T helper epitope may be inserted within a repeat region for example C-LytA repeats₂₋₅_{_{—}}-C-LytA repeat 6a-P₂ epitope—C-LytA repeat 6b, where the P2 epitope is inserted within the sixth repeat (see FIG. 2).

In other preferred embodiments the C-terminal end of CPL1 (C-CPL1) may be used as an alternative to C-LytA.

Alternatively, the P2 epitope in the above constructs may be replaced by other promiscuous T epitopes, for example P30. In an embodiment of the invention, two or more promiscuous epitopes are part of the fusion construct. It is however preferred to keep the fusion partner as small as possible, thus limiting the number of potentially interfering CD8+ and B epitopes. Thus the fusion partner is preferably no bigger than 100-140 amino acids, preferably no bigger than 120 amino acids, typically about 100 amino acid.

The antigen to which the fusion partner is fused may be from bacterial, viral, protozoan, fungal or mammalian, including human, sources.

The fusion partner of the present invention are preferably fused to a self antigen such as a tumour associated or tissue specific antigens such as those for prostrate, breast, colorectal, lung, pancreatic, ovarian, renal or melanoma cancers. Fragments of said self or tumour antigens are expressly contemplated to be fused to the fusion partner of the invention. Typically the fragment will contain at least 20, preferably 50, more preferably 100 contiguous amino acids of the full-length sequence. Typically such fragments will be devoid of one or more transmembrane domains or may have N-terminal or C-terminal deletions of about 3, 5, 8, 10, 15, 20, 28, 33, 50, 54 amino acids. Such fragments will, when suitably presented, be able to generate immune responses that recognise the full length protein. Particularly illustrative polypeptides of the present invention comprise a sequence of at least 10 contiguous amino acids, preferably 20, more preferably 30, 40, 50, 60, 70, 80, 90, 100, 110, 120, 130, 140, 150, 160, 170, 180 amino acids of a tumour associated or tissue specific protein fused to the fusion partner.

The polypeptides of the invention are immunogenic, i.e., they react detectably within an immunoassay (such as an ELISA or T-cell stimulation assay) with antisera and/or T-cells from a patient with cripto expressing cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilised on a solid support and contacted with patient sera to allow binding of antibodies within the sera to the immobilised polypeptide. Unbound sera may then be removed and bound antibodies detected using, for example, ¹²⁵I-labeled Protein A. As would be recognised by the skilled artisan, immunogenic portions of tumour associated or tumour specific antigen are also encompassed by the present invention. An “immunogenic portion” as used herein, is a fragment that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques. In one preferred embodiment, an immunogenic portion of a polypeptide is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., about 1-50 amino acids, preferably about 1-30 amino acids, more preferably about 5-15 amino acids), relative to the mature protein.

Exemplary antigens or fragments derived therefrom include MAGE 1, Mage 3 and MAGE 4 or other MAGE antigens such as disclosed in WO 99/40188, PRAME (WO 96/10577), BAGE, RAGE, LAGE 1 (WO 98/32855), LAGE 2 (also known as NY-ESO-1, WO 98/14464), XAGE (Liu et al, Cancer Res, 2000, 60:4752-4755; WO 02/18584) SAGE, and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma.

In a preferred embodiment prostate antigens are utilised, such as Prostate specific antigen (PSA), PAP, PSCA (PNAS 95(4) 1735-1740 1998), PSMA or the antigen known as prostase.

In a particularly preferred embodiment, the prostate antigen is P501S or a fragment thereof. P501S, also named prostein (Xu et al., Cancer Res. 61, 2001, 1563-1568), is known as SEQ ID NO. 113 of WO98/37814 and is a 553 amino acid protein. Immunogenic fragments and portions thereof comprising at least 20, preferably 50, more preferably 100 contiguous amino acids as disclosed in the above referenced patent application and are specifically contemplate by the present invention. Preferred fragments are disclosed in WO 98/50567 (PS108 antigen) and as prostate cancer-associated protein (SEQ ID NO: 9 of WO 99/67384). Other preferred fragments are amino acids 51-553, 34-553 or 55-553 of the full-length P501S protein. In particular, construct 1, 2 and 3 (see FIG. 2, SEQ ID NOs. 27-32) are expressly contemplated, and can be expressed in yeast systems, for example DNA sequences encoding such polypeptides can be expressed in yeast system.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Iinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed. Prostase nucleotide sequence and deduced polypeptide sequence and homologous are disclosed in Ferguson, et al. (Proc. Natl. Acad. Sci. USA 1999, 96, 3114-3119) and in International Patent Applications No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

Other prostate specific antigens are known from WO98/37418, and WO/004149. Another is STEAP (PNAS 96 14523 14528 7-12 1999).

Other tumour associated antigens useful in the context of the present invention include: Plu −1 J. Biol. Chem 274 (22) 15633-15645, 1999, HASH-1, HASH-2 (Alders, M. et al., Hum. Mol. Genet. 1997, 6, 859-867), Cripto (Salomon et al Bioessays 199, 21 61-70, U.S. Pat. No. 5,654,140), CASB616 (WO 00/53216), Criptin (U.S. Pat. No. 5,981,215). Additionally, antigens particularly relevant for vaccines in the therapy of cancer also comprise tyrosinase, telomerase, P53, NY-Br1.1 (WO 01/47959) and fragments thereof such as disclosed in WO 00/43420, B726 (WO 00/60076, SEQ ID nos 469 and 463; WO 01/79286, SEQ ID nos 474 and 475), P510 (WO 01/34802 SEQ ID nos 537 and 538) and survivin.

The present invention is also useful in combination with breast cancer antigens such as Her-2/neu, mammaglobin (U.S. Pat. No. 5,668,267), B305D (WO 00/61753 SEQ ID nos 299, 304, 305 and 315), or those disclosed in WO 00/52165, WO 99/33869, WO 99/19479, WO 98/45328. Her-2/neu antigens are disclosed inter alia, in U.S. Pat. No. 5,801,005. Preferably the Her-2/neu comprises the entire extracellular domain (comprising approximately amino acid 1-645) or fragments thereof and at least an immunogenic portion of or the entire intracellular domain approximately the C terminal 580 amino acids. In particular, the intracellular portion should comprise the phosphorylation domain or fragments thereof. Such constructs are disclosed in WO 00/44899. A particularly preferred construct is known as ECD-PhD, a second is known as ECD deltaPhD (see WO 00/44899). The Her-2/neu as used herein can be derived from rat, mouse or human.

Certain tumour antigens are small peptide antigens (ie less than about 50 amino acids). These antigens can be chemically conjugated to the modified choline binding protein of the present invention.

Exemplary peptides included Mucin derived peptides such as MUC-1 (see for example U.S. Pat. No. 5,744,144; U.S. Pat. No. 5,827,666; WO 88/05054, U.S. Pat. No. 4,963,484). Specifically contemplated are MUC-1 derived peptides that comprise at least one repeat unit of the MUC-1 peptide, preferably at least two such repeats and which is recognised by the SM3 antibody (U.S. Pat. No. 6,054,438). Other mucin derived peptides include peptide from MUC-5.

Alternatively, said antigen is an interleukin such as IL13 and IL14, which are preferred. Or said antigen maybe a self peptide hormone such as whole length Gonadotrophin hormone releasing hormone (GnRH, WO 95/20600), a short 10 amino acid long peptide, useful in the treatment of many cancers, or in immunocastration.

Other tumour-specific antigens are suitable to be coupled with the modified Choline binding protein of the present invention include, but are not restricted to tumour-specific gangliosides such as GM2, and GM3.

The covalent coupling of the peptide to modified choline binding protein can be carried out in a manner well known in the art. Thus, for example, for direct covalent coupling it is possible to utilise a carbodiimide, glutaraldehyde or (N-[γ-maleimidobutyryloxy]succinimide ester, utilising common commercially available heterobifunctional linkers such as CDAP and SPDP (using manufacturers instructions). After the coupling reaction, the immunogen can easily be isolated and purified by means of a dialysis method, a gel filtration method, a fractionation method etc.

The antigen may also be derived from sources which are pathogenic to humans, such as such as Human Immunodeficiency virus HIV-1 (such as tat, nef, reverse transcriptase, gag, gp120 and gp160), human herpes simplex viruses, such as gD or derivatives thereof or Immediate Early protein such as ICP27 from HSV1 or HSV2, cytomegalovirus ((esp Human)(such as gB or derivatives thereof, Rotavirus (including live-attenuated viruses), Epstein Barr virus (such as gp350 or derivatives thereof), Varicella Zoster Virus (such as gpl, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, . . . ), flaviviruses (e.g. Yellow Fever Virus, Dengue Virus, Tick-borne encephalitis virus, Japanese Encephalitis Virus) or Influenza virus (whole live or inactivated virus, split influenza virus, grown in eggs or MDCK cells, or whole flu virosomes (as described by R. Gluck, Vaccine, 1992, 10, 915-920) or purified or recombinant proteins thereof, such as HA, NP, NA, or M proteins, or combinations thereof), or derived from bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Usteria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carini; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Most preferred antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Preferred bacterial antigens are derived from Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial antigens are derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy varients or fusion proteins thereof.

Derivatives of Hepatitis B Surface antigen are well known in the art and include, inter alia, those PreS1, PreS2 S antigens set forth described in European Patent applications EP-A-414 374; EP-A-0304 578, and EP 198474. In one preferred The HBV antigen is HBV polymerase (Ji Hoon Jeong et al, 1996, BBRC 223, 264-271; Lee H. J. et al, Biotechnol. Lett. 15, 821-826). In another preferred aspect the antigen within the fusion is a HIV-1 antigen, gp120, especially when expressed in CHO cells. In a further embodiment, antigen comprises gD2t as hereinabove defined.

In a preferred embodiment of the present invention fusions comprise an antigen derived from the Human Papilloma Virus (HPV 6a, 6b, 11, 16, 18, 31, 33, 35, 39, 45, 51, 52, 56, 58, 59 and 68), in particular those HPV serotypes considered to be responsible for genital warts (HPV 6 or HPV 11 and others), and the HPV viruses responsible for cervical cancer (HPV16, HPV18 and others).

Suitable HPV antigens are E1, E2, E4, E5, E6, E7, L1 and L2. Particularly preferred forms of genital wart prophylactic, or therapeutic, fusions comprise L1 particles or capsomers, and fusion proteins comprising one or more antigens selected from the HPV 6 and HPV 11 proteins E6, E7, L1, and L2.

The most preferred forms of fusion protein are: L2E7 as disclosed in WO 96/26277, and proteinD(1/3)-E7 disclosed in GB 9717953.5 (PCT/EP98/05285).

A preferred HPV cervical infection or cancer, prophylaxis or therapeutic vaccine, composition may comprise HPV 16 or 18 antigens. For example, L1 or L2 antigen monomers, or L1 or L2 antigens presented together as a virus like particle (VLP) or the L1 alone protein presented alone in a VLP or caposmer structure. Such antigens, virus like particles and capsomer are per se known. See for example WO94/00152, WO94/20137, WO94/05792, and WO93/02184.

Additional early proteins may be included alone or as fusion proteins such as E7, E2 or preferably E5 for example; particularly preferred embodiments of this includes a VLP comprising L1E7 fusion proteins (WO 96/11272). Particularly preferred HPV 16 antigens comprise the early proteins E6 or E7 in fusion with a protein D carrier to form Protein D—E6 or E7 fusions from HPV 16, or combinations thereof; or combinations of E6 or E7 with L2 (WO 96/26277).

Alternatively the HPV 16 or 18 early proteins E6 and E7, may be presented in a single molecule, preferably a Protein D—E6/E7 fusion. Other fusions optionally contain either or both E6 and E7 proteins from HPV 18, preferably in the form of a Protein D—E6 or Protein D—E7 fusion protein or Protein D E6/E7 fusion protein. Fusions may comprise antigens from other HPV strains, preferably from strains HPV 31 or 33.

Fusions according to the present invention comprise antigens derived from parasites that cause Malaria. For example, preferred antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in the International Patent Application No. PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. A preferred embodiment of the present invention is a fusion wherein the antigenic preparation comprises a combination of the RTS,S and TRAP antigens. Other plasmodia antigens that are likely candidates to be components of the fusion are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

The present invention also provides a polynucleotide encoding the fusion partner according to the present invention. The invention further relates a polynucleotide that hybridise to the polynucleotide sequence provided herein in FIG. 1 (SEQ ID NO:9 to 16). In this regard, the invention especially relates to polynucleotides that hybridise under stringent conditions to the polynucleotide described herein. As herein used, the terms “stringent conditions” and “stringent hybridisation conditions” mean hybridisation occurring only if there is at least 95% and preferably at least 97% identity between the sequences. A specific example of stringent hybridization conditions is overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 micrograms/ml of denatured, sheared salmon sperm DNA, followed by washing the hybridisation support in 0.1×SSC at about 65° C. Hybridisation and wash conditions are well known and exemplified in Sambrook, et al., Molecular Cloning: A Laboratory Manual, Second Edition, Cold Spring Harbor, N.Y., (1989), particularly Chapter 11 therein. Solution hybridisation may also be used with the polynucleotide sequences provided by the invention.

The present invention also provides a polynucleotide encoding the polypeptide comprising the fusion partner according to the present invention fused to a tumour associated antigen or fragment thereof. In particular, the present invention provides for polynucleotide sequences encoding a fusion partner protein comprising a choline binding domain and a heterologous promiscuous T heper epitope, preferably wherein the choline binding domain is derived from the C terminus of LytA. In a more preferred embodiment, the C-LytA moiety of the polynucleotides according to the invention comprise at least four repeats of any of SEQ ID NO.9-14, more preferably comprise the sequence of SEQ ID NO.15, still more preferably the sequence of SEQ ID NO.16. In other related embodiments, the present invention provides for polynucleotide variants having substantial identity to the sequences disclosed herein in SEQ ID NOs:9-16, for example those comprising at least 70% sequence identity, preferably at least 75%, 80%, 85%, 90%, 95%, 96%, 97%, 98%, or 99% or higher, sequence identity compared to a polynucleotide sequence of this invention using conventional methods, e.g., BLAST analysis using standard parameters. In a still further embodiment the polynucleotide as claimed further comprises a heterologous protein.

Such polynucleotide sequences can be inserted into a suitable expression vector and expressed in a suitable host. Vectors may be provided which encode the modified choline binding protein of the invention and which contain a suitable restriction site into which a DNA encoding a poorly immunogenic protein can be inserted to produce a fusion protein. In other embodiments of the invention, polynucleotide sequences or fragments thereof which encode polypeptide fusions of the invention, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. The DNA code has 4 letters (A, T, C and G) and uses these to spell three letter “codons” which represent the amino acids the proteins encodes in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino add, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. It is believed that the presence in a heterologous DNA sequence of clusters of codons which are rarely observed in the host in which expression is to occur, is predictive of low heterologous expression levels in that host.

In consequence, codons preferred by a particular prokaryotic (for example E. coli or yeast) or eukaryotic host can be optimised, that is selected to increase the rate of protein expression, to produce a recombinant RNA transcript having desirable properties, such as for example a half-life which is longer than that of a transcript generated from the naturally occurring sequence, or to optimise the immune response in humans. The process of codon optimisation may include any sequence, generated either manually or by computer software, where some or all of the codons of the native sequence are modified. Several methods have been published (Nakamura et. al., Nucleic Acids Research 1996, 24:214-215; WO98/34640). One preferred method according to this invention is Syngene method, a modification of Calcgene method (R. S. Hale and G Thompson (Protein Expression and Purification Vol. 12 pp. 185-188 (1998)).

Accordingly in a preferred embodiment the DNA sequence of the protein has a RSCU (Relative synomons Codon useage (also known as Codon Index CI)) of at least 0.65 and have less than 85% identity to the corresponding wild type region.

This process of codon optimisation and the resulting constructs are advantageous as they may have some or all of the following benefits: 1) to improve expression of the gene product by replacing rare or infrequently used codons with more frequently used codons, 2) to remove or include restriction enzyme sites to facilitate downstream cloning and 3) to reduce the potential for homologous recombination between the insert sequence in the DNA vector and genomic sequences and 4) to improve the immune response in humans by raising a cellular and/or an antibody response (preferably both responses) against the target antigen. The sequences of the present invention advantageously have reduced recombination potential, but express to at least the same level as the wild type sequences. Due to the nature of the algorithms used by the SynGene programme to generate a codon optimised sequence, it is possible to generate an extremely large number of different codon optimised sequences which will perform a similar function. In brief, the codons are assigned using a statistical method to give synthetic gene having a codon frequency closer to that found naturally in highly expressed E. coli and human genes. In brief, the codons are assigned using a statistical method to give synthetic gene having a codon frequency closer to that found naturally in highly expressed human genes such as β-Actin. Illustrative, although non limiting, examples of suitable codon-optimised sequences are given in SEQ ID NOs:19-22 and SEQ ID NOs:24-26.

In the polynucleotides of the present invention, the codon usage pattern is altered from that typical of the target antigen to more closely represent the codon bias of a highly expressed gene in a target organism, for example human β-actin. The “codon usage coefficient” is a measure of how closely the codon pattern of a given polynucleotide sequence resembles that of a target species. Codon frequencies can be derived from literature sources for the highly expressed genes of many species (see e.g. Nakamura et. al. Nucleic Acids Research 1996, 24:214-215). The codon frequencies for each of the 61 codons (expressed as the number of occurrences occurrence per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species.

According to the present invention, the codon usage pattern of the polynucleotide will preferably exclude codons representing <10% of the codons used for a particular amino acid. A relative synonymous codon usage (RSCU) value is the observed number of codons divided by the number expected if all codons for that amino acid were used equally frequently. A polynucleotide of the present invention will preferably exclude codons with an RSCU value of less than 0.2 in highly expressed genes of the target organism. A polynucleotide of the present invention will generally have a codon usage coefficient for highly expressed human genes of greater than 0.6, preferably greater than 0.65, most preferably greater than 0.7. Codon usage tables for human can also be found in Genbank.

In comparison, a highly expressed beta actin gene has a RSCU of 0.747.

The codon usage table (Table 1) for a homo sapiens is set out below:

TABLE 1
Codon usage for human (highly expressed)
genes Jan. 24, 1991 (human_high.cod)

| AmAcid | Codon | Number | /1000 | Fraction |
|---|---|---|---|---|
| Gly | GGG | 905.00 | 18.76 | 0.24 |
| Gly | GGA | 525.00 | 10.88 | 0.14 |
| Gly | GGT | 441.00 | 9.14 | 0.12 |
| Gly | GGC | 1867.00 | 38.70 | 0.50 |
| Glu | GAG | 2420.00 | 50.16 | 0.75 |
| Glu | GAA | 792.00 | 16.42 | 0.25 |
| Asp | GAT | 592.00 | 12.27 | 0.25 |
| Asp | GAC | 1821.00 | 37.75 | 0.75 |
| Val | GTG | 1866.00 | 38.68 | 0.64 |
| Val | GTA | 134.00 | 2.78 | 0.05 |
| Val | GTT | 198.00 | 4.10 | 0.07 |
| Val | GTC | 728.00 | 15.09 | 0.25 |
| Ala | GCG | 652.00 | 13.51 | 0.17 |
| Ala | GCA | 488.00 | 10.12 | 0.13 |
| Ala | GCT | 654.00 | 13.56 | 0.17 |
| Ala | GCC | 2057.00 | 42.64 | 0.53 |
| Arg | AGG | 512.00 | 10.61 | 0.18 |
| Arg | AGA | 298.00 | 6.18 | 0.10 |
| Ser | AGT | 354.00 | 7.34 | 0.10 |
| Ser | AGC | 1171.00 | 24.27 | 0.34 |
| Lys | AAG | 2117.00 | 43.88 | 0.82 |
| Lys | AAA | 471.00 | 9.76 | 0.18 |
| Asn | AAT | 314.00 | 6.51 | 0.22 |
| Asn | AAC | 1120.00 | 23.22 | 0.78 |
| Met | ATG | 1077.00 | 22.32 | 1.00 |
| Ile | ATA | 88.00 | 1.82 | 0.05 |
| Ile | ATT | 315.00 | 6.53 | 0.18 |
| Ile | ATC | 1369.00 | 28.38 | 0.77 |
| Thr | ACG | 405.00 | 8.40 | 0.15 |
| Thr | ACA | 373.00 | 7.73 | 0.14 |
| Thr | ACT | 358.00 | 7.42 | 0.14 |
| Thr | ACC | 1502.00 | 31.13 | 0.57 |
| Trp | TGG | 652.00 | 13.51 | 1.00 |
| End | TGA | 109.00 | 2.26 | 0.55 |
| Cys | TGT | 325.00 | 6.74 | 0.32 |
| Cys | TGC | 706.00 | 14.63 | 0.68 |
| End | TAG | 42.00 | 0.87 | 0.21 |
| End | TAA | 46.00 | 0.95 | 0.23 |
| Tyr | TAT | 360.00 | 7.46 | 0.26 |
| Tyr | TAC | 1042.00 | 21.60 | 0.74 |
| Leu | TTG | 313.00 | 6.49 | 0.06 |
| Leu | TTA | 76.00 | 1.58 | 0.02 |
| Phe | TTT | 336.00 | 6.96 | 0.20 |
| Phe | TTC | 1377.00 | 28.54 | 0.80 |
| Ser | TCG | 325.00 | 6.74 | 0.09 |
| Ser | TCA | 165.00 | 3.42 | 0.05 |
| Ser | TCT | 450.00 | 9.33 | 0.13 |
| Ser | TCC | 958.00 | 19.86 | 0.28 |
| Arg | CGG | 611.00 | 12.67 | 0.21 |
| Arg | CGA | 183.00 | 3.79 | 0.06 |
| Arg | CGT | 210.00 | 4.35 | 0.07 |
| Arg | CGC | 1086.00 | 22.51 | 0.37 |
| Gln | CAG | 2020.00 | 41.87 | 0.88 |
| Gln | CAA | 283.00 | 5.87 | 0.12 |
| His | CAT | 234.00 | 4.85 | 0.21 |
| His | CAC | 870.00 | 18.03 | 0.79 |
| Leu | CTG | 2884.00 | 59.78 | 0.58 |
| Leu | CTA | 166.00 | 3.44 | 0.03 |
| Leu | CTT | 238.00 | 4.93 | 0.05 |
| Leu | CTC | 1276.00 | 26.45 | 0.26 |
| Pro | CCG | 482.00 | 9.99 | 0.17 |
| Pro | CCA | 456.00 | 9.45 | 0.16 |
| Pro | CCT | 568.00 | 11.77 | 0.19 |
| Pro | CCC | 1410.00 | 29.23 | 0.48 |

A DNA sequence encoding the fusion proteins or modified choline binding protein of the present invention can be synthesised using standard DNA synthesis techniques, such as by enzymatic ligation as described by D. M. Roberts et al. in Biochemistry 1985, 24, 5090-5098, by chemical synthesis, by in vitro enzymatic polymerisation, or by PCR technology utilising for example a heat stable polymerase, or by a combination of these techniques.

Enzymatic polymerisation of DNA may be carried out in vitro using a DNA polymerase such as DNA polymerase I (Klenow fragment) or Taq polymerase in an appropriate buffer containing the nucleoside triphosphates dATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer, such as 0.05M Tris (pH 7.4), 0.01M MgCl₂, 0.01M dithiothreitol, 1 mM spermidine, 1 mM ATP and 0.1 mg/ml bovine serum albumin, at a temperature of 4° C. to ambient, generally in a volume of 50 μl or less. The chemical synthesis of the DNA polymer or fragments may be carried out by conventional phosphotriester, phosphate or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat, and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus, and H. Koester, Nucleic Acids Research, 1984, 12, 4539; and H. W. D. Matthes et al., EMBO Journal, 1984, 3, 801.

The process of the invention may be performed by conventional recombinant techniques such as described in Maniatis et al., Molecular Cloning—A Laboratory Manual; Cold Spring Harbor, 1982-1989.

In particular, the process may comprise the steps of:

i) preparing a replicable or integrating expression vector capable, in a host cell, of expressing a DNA polymer comprising a nucleotide sequence that encodes the protein or an immunogenic derivative thereof

ii) transforming a host cell with said vector

iii) culturing said transformed host cell under conditions permitting expression of said DNA polymer to produce said protein; and

iv) recovering said protein

The term ‘transforming’ is used herein to mean the introduction of foreign DNA into a host cell. This can be achieved for example by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest.

The expression vectors are novel and also form part of the invention.

The replicable expression vectors may be prepared in accordance with the invention, by cleaving a vector compatible with the host cell to provide a linear DNA segment having an intact replicon, and combining said linear segment with one or more DNA molecules which, together with said linear segment encode the desired product, such as the DNA polymer encoding the protein of the invention, or derivative thereof, under ligating conditions.

Thus, the DNA polymer may be performed or formed during the construction of the vector, as desired.

The choice of vector will be determined in part by the host cell, which may be prokaryotic or eukaryotic but are preferably E. coli, yeast or CHO cells. Suitable vectors include plasmids, bacteriophages, cosmids and recombinant viruses. Expression and cloning vectors preferably contain a selectable marker such that only the host cells expressing the marker will survive under selective conditions. Selection genes include but are not limited to the one encoding protein that confer a resistance to ampicillin, tetracyclin or kanamycin. Expression vectors also contain control sequences which are compatible with the designated host. For example, expression control sequences for E. coli, and more generally for prokaryotes, include promoters and ribosome binding sites. Promoter sequences may be naturally occurring, such as the β-lactamase (penicillinase) (Weissman 1981, In Interferon 3 (ed. L. Gresser), lactose (lac) (Chang et al. Nature, 1977, 198: 1056) and tryptophan (trp) (Goeddel et al. Nucl. Acids Res. 1980, 8, 4057) and lambda-derived P_{L} promoter system. In addition, synthetic promoters which do not occur in nature also function as bacterial promoters. This is the case for example for the tac synthetic hybrid promoter which is derived from sequences of the trp and lac promoters (De Boer et al., Proc. Natl. Acad. Sci. USA 1983, 80, 21-26). These systems are particularly suitable with E. coli.

Yeast compatible vectors also carry markers that allow the selection of successful transformants by conferring prototrophy to auxotrophic mutants or resistance to heavy metals on wild-type strains. Expression control sequences for yeast vectors include promoters for glycolytic enzymes (Hess et al., J. Adv. Enzyme Reg. 1968, 7, 149), PHO5 gene encoding acid phosphatase, CUP1 gene, ARG3 gene, GAL genes promoters and synthetic promoter sequences. Other control elements useful in yeast expression are terminators and mRNA leader sequences. The 5′ coding sequence is particularly useful since it typically encodes a signal peptide comprised of hydrophobic amino acids which direct the secretion of the protein from the cell. Suitable signal sequences can be encoded by genes for secreted yeast proteins such as the yeast invertase gene and the α-factor gene, acid phosphatase, killer toxin, the alpha-mating factor gene and recently the heterologous inulinase signal sequence derived from INULA gene of Kluyveromyces marxianus. Suitable vectors have been developed for expression in Pichia pastoris and Saccharomyces cerevisiae.

A variety of P. pastoris expression vectors are available based on various inducible or constitutive promoters (Cereghino and Cregg, FEMS Microbiol. Rev. 2000, 24:45-66). For the production of cytosolic and secreted proteins, the most commonly used P. pastoris vectors contain the very strong and tightly regulated alcohol oxidase (AOX1) promoter. The vectors also contain the P. pastoris histidinol dehydrogenase (HIS4) gene for selection in his4 hosts. Secretion of foreign protein require the presence of a signal sequence and the S. cerevisiae prepro alpha mating factor signal sequence has been widly and successfully used in Pichia expression system. Expression vectors are integrated into the P. pastoris genome to maximize the stability of expression strains. As in S. cerevisiae, cleavage of a P. pastoris expression vector within a sequence shared by the host genome (AOX1 or HIS4) stimulates homologous recombination events that efficiently target integration of the vector to that genomic locus. In general, a recombinant strain that contains multiple integrated copies of an expression cassette can yield more heterologous protein than single-copy strain. The most effective way to obtain high copy number transformants requires the transformation of Pichia recipient strain by the sphaeroplast technique (Cregg et all 1985, Mol. Cell. Biol. 5: 3376-3385).

The preparation of the replicable expression vector may be carried out conventionally with appropriate enzymes for restriction, polymerisation and ligation of the DNA, by procedures described in, for example, Maniatis et al. cited above.

The recombinant host cell is prepared, in accordance with the invention, by transforming a host cell with a replicable expression vector of the invention under transforming conditions. Suitable transforming conditions are conventional and are described in, for example, Maniatis et al. cited above, or “DNA Cloning” Vol. II, D. M. Glover ed., IRL Press Ltd, 1985.

The choice of transforming conditions depends upon the choice of the host cell to be transformed. For example, in vivo transformation using a live viral vector as the transforming agent for the polynucleotides of the invention is described above. Bacterial transformation of a host such as E. coli may be done by direct uptake of the polynucleotides (which may be expression vectors containing the desired sequence) after the host has been treated with a solution of CaCl₂ (Cohen et al., Proc. Nat. Acad. Sci., 1973, 69, 2110) or with a solution comprising a mixture of rubidium chloride (RbC1), MnCl₂, potassium acetate and glycerol, and then with 3-[N-morpholino]-propane-sulphonic acid, RbC1 and glycerol or by electroporation. Transformation of lower eukaryotic organisms such as yeast cells in culture by direct uptake may be carried out for example by using the method of Hinnen et al (Proc. Natl. Acad. Sci. 1978, 75:1929-1933). Mammalian cells in culture may be transformed using the calcium phosphate co-precipitation of the vector DNA onto the cells (Graham & Van der Eb, Virology 1978, 52, 546). Other methods for introduction of polynucleotides into mammalian cells include dextran mediated transfection, polybrene mediated transfection, protoplast fusion, electroporation, encapsulation of the polynucleotide(s) into liposomes, and direct micro-injection of the polynucleotides into nuclei.

The invention also extends to a host cell transformed with a nucleic acid encoding the protein of the invention or a replicable expression vector of the invention.

Culturing the transformed host cell under conditions permitting expression of the DNA polymer is carried out conventionally, as described in, for example, Maniatis et al. and “DNA Cloning” cited above. Thus, preferably the cell is supplied with nutrient and cultured at a temperature below 50° C., preferably between 25° C. and 42° C., more preferably between 25° C. and 35° C., most preferably at 30° C. The incubation time may vary from a few minutes to a few hours, according to the proportion of the polypeptide in the bacterial cell, as assessed by SDS-PAGE or Western blot.

The product may be recovered by conventional methods according to the host cell and according to the localisation of the expression product (intracellular or secreted into the culture medium or into the cell periplasm). Thus, where the host cell is bacterial, such as E. coli it may, for example, be lysed physically, chemically or enzymatically and the protein product isolated from the resulting lysate. Where the host cell is mammalian, the product may generally be isolated from the nutrient medium or from cell free extracts. Where the host cell is a yeast such as Saccharomyces cerevisiae or Pichia pastoris, the product may generally be isolated from from lysed cells or from the culture medium, and then further purified using conventional techniques. The specificity of the expression system may be assessed by western blot or by ELISA using an antibody directed against the polypeptide of interest.

Conventional protein isolation techniques include selective precipitation, adsorption chromatography, and affinity chromatography including a monoclonal antibody affinity column. When the proteins of the present invention are expressed with a histidine tail (His tag), they can easily be purified by affinity chromatography using an ion metal affinity chromatography column (IMAC) column. The metal ion, may be any suitable ion for example zinc, nickel, iron, magnesium or copper, but is preferably zinc or nickel. Preferably the IMAC buffer contains detergent, preferably an anionic detergent such as SDS, more preferably a non-ionic detergent such as Tween 80, or a zwitterionic detergent such as Empigen BB, as this may result in lower levels of endotoxin in the final product.

Further chromatographic steps include for example a Q-Sepharose step that may be operated either before of after the IMAC column. Preferably the pH is in the range of 7.5 to 10, more preferably from 7.5 to 9.5, optimally between 8 and 9.

The proteins of the invention can thus be purified according to the following protocol. After cell disruption, cell extracts containing the protein can be solubilised in a pH 8.5 Tris buffer containing urea (8.0 M for example), and SDS (from 0.5% to 1% for example). After centrifugation, the resulting supernatant may then be loaded onto on to an IMAC (Nickel) Sepharose FF column equilibrated with a pH 8.5 Tris buffer. The column may then be washed with a high salt containing buffer (eg 0.75-1.5 m NaC1, 15 mM pH 8.5 Tris buffer). The column may optionally then be washed again with phosphate buffer without salt. The proteins of the invention may be eluated from the column with an imidazole-containing buffered solution. The proteins can then be submitted to an additional chromatographic step, such as to an anion exchange chromatography (Q Sepharose for example).

The proteins of the present invention are provided either soluble in a liquid form or in a lyophilised form, which is the preferred form. It is generally expected that each human dose will comprise 1 to 1000 μg of protein, and preferably 30-300 μg. The purification process can also include a carboxyamidation step whereby the protein is first reduced in the presence of Glutathion and then carboxymethylated in the presence of iodoacetamide. This step offers the advantage of controling the oxidative aggregation of the molecule with itself or with host cell protein contaminants through covalent bridging with disulphide bonds.

The present invention also provides pharmaceutical and immunogenic compositions comprising a protein of the present invention in a pharmaceutically acceptable excipient. A preferred vaccine composition comprises at least a protein according to the invention. Said protein has, preferably, blocked thiol groups and is highly purified, e.g. has less than 5% host cell contamination. Such vaccine may optionally contain one or more other tumour-associated antigen and derivatives. For example, suitable other associated antigen include prostase, PAP-1, PSA (prostate specific antigen), PSMA (prostate-specific membrane antigen), PSCA (Prostate Stem Cell Antigen), STEAP.

In another embodiment, illustrative immunogenic compositions, such as for example vaccine compositions, of the present invention comprise DNA encoding one or more of the fusion polypeptides as described above, such that the fusion polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476). Since humans are sometimes infected by common human adenovirus serotypes such as AdHu5, a significant proportion of the population have a neutralizing antibody response to the adenovirus, which is likley to effect the immune response to a heterologous antigen in a recombinant vaccine based system. Non-human primate adenoviral vectors such as the chimpanzee adenovirus 68 (AdC68, Fitzgerald et al. (2003) J. Immunol 170(3):1416-22)) are may offer an alternative adenoviral system without the disadvantage of a pre-existing neutralising antibody response.

Various adeno-associated virus (MV) vector systems have also been developed for polynucleotide delivery. MV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the nucleic acid molecules encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

The compositions of the present invention can be delivered by a number of routes such as intramuscularly, subcutaneously, intraperitonally or intravenously.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells. In a preferred embodiment, the composition is delivered intradermally. In particular, the composition is delivered by means of a gene gun (particularly particle bombardment) administration techniques which involve coating the vector on to a bead (eg gold) which are then administered under high pressure into the epidermis; such as, for example, as described in Haynes et al, J Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. However, this treatment regime will be significantly varied depending upon the size of the patient, the disease which is being treated/protected against, the amount of nucleotide sequence administered, the route of administration, and other factors which would be apparent to a skilled medical practitioner.

It is therefore another aspect of the present invention to provide for the use of a protein or a DNA encoding said protein, as described herein, in the manufacture of an immunogenic composition for eliciting an immune response in a patient. Preferably the immune response is to be elicited by sequential administration of i) the said protein followed by the said DNA sequence; or ii) the said DNA sequence followed by the said protein. More preferably the DNA sequence is coated onto biodegradable beads or delivered via a particle bombardment approach. Still more preferably the protein ios adjuvanted, preferably with a TH-1 inducing adjuvant, preferably with a CpG/QS21 based adjuvant formulation.

The vectors which comprise the nucleotide sequences encoding antigenic peptides are administered in such amount as will be prophylactically or therapeutically effective. The quantity to be administered, is generally in the range of one picogram to 16 milligram, preferably 1 picogram to 10 micrograms for particle-mediated delivery, and 10 micrograms to 16 milligram for other routes of nucleotide per dose. The exact quantity may vary considerably depending on the weight of the patient being immunised and the route of administration.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

The fusion proteins and encoding polypeptides according to the invention can also be formulated as a pharmaceutical/immunogenic composition, e.g. as a vaccine. Accordingly therefore, the present invention also provides for a pharmaceutical/immunogenic composition comprising a fusion protein of the present invention in a pharmaceutically acceptable excipient. Accordingly there is also provided a process for the preparation of an immunogenic composition according to the present invention, comprising admixing the fusion protein of the invention or the encoding polynucleotide with a suitable adjuvant, diluent or other pharmaceutically acceptable carrier.

The fusion proteins of the present invention are provided preferably at least 80% pure more preferably 90% pure as visualised by SDS PAGE. Preferably the proteins appear as a single band by SDS PAGE.

Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877.

The fusion proteins of the present invention and encoding polynucleotides are preferably adjuvanted in the vaccine formulation of the invention. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol 7:145-173, 1989.

Preferred TH-1 inducing adjuvants are selected from the group of adjuvants comprising: 3D-MPL, QS21, a mixture of QS21 and cholesterol, and a CpG oligonucleotide or a mixture of two or more said adjuvants. Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as CarbopoIR to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 as disclosed in WO 00/09159 and in WO 00/62800. Preferably the formulation additionally comprises an oil in water emulsion and tocopherol.

In a yet further embodiment the present invention provides an immunogenic composition comprising a fusion protein according to the invention, and further comprising D3-MPL, a saponin preferably QS21 and a CpG oligonucleotide, optionally formulated in an oil in water emulsion.

Additional illustrative adjuvants for use in the pharmaceutical compositions of the invention include Montanide ISA 720 (Seppic, France), SAF (Chiron, California, United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula (I): HO(CH₂CH₂O)ₙ-A-R, wherein, n is 1-50, A is a bond or —C(O)—, R is C₁₋₅₀ alkyl or Phenyl C₁₋₅₀ alkyl. One embodiment of the present invention consists of a vaccine formulation comprising a polyoxyethylene ether of general formula (I), wherein n is between 1 and 50, preferably 4-24, most preferably 9; the R component is C₁₋₁₅₀, preferably C₄-C₂₀ alkyl and most preferably C₁₋₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549. The polyoxyethylene ether according to the general formula (I) above may, if desired, be combined with another adjuvant. For example, a preferred adjuvant combination is preferably with CpG as described in the pending UK patent application GB 9820956.2.

It is an embodiment of the invention that the antigens, including nucleic acid vector, of the invention be utilised with immunostimulatory agent. Preferably the immunostimulatory agent is administered at the same time as the antigens of the invention and in preferred embodiments are formulated together. It is another embodiment of the invention that the antigen and immunostimulatory agent (or vice versa) are administered sequentially to the same or adjacent sites, separated in time by periods of between 0-100 hours. Such immunostimulatory agents include but are not limited to: synthetic imidazoquinolines such as imiquimod [S-26308, R-837], (Harrison, et al., Vaccine 19: 1820-1826, 2001; and resiquimod [S-28463, R-848] (Vasilakos, et al., Cellular immunology 204: 64-74, 2000.; Schiff bases of carbonyls and amines that are constitutively expressed on antigen presenting cell and T-cell surfaces, such as tucaresol (Rhodes, J. et al., Nature 377: 71-75, 1995), cytokine, chemokine and co-stimulatory molecules as either protein or peptide, including for example pro-inflammatory cytokines such as Interferon, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., Vaccine 19: 3778-3786, 2001) squalene, alpha-tocopherol, polysorbate 80, DOPC and cholesterol, endotoxin, [LPS], (Beutler, B., Current Opinion in Microbiology 3: 23-30, 2000); CpG oligo- and di-nucleotides (Sato, Y. et al., Science 273 (5273): 352-354, 1996; Hemmi, H. et al., Nature 408: 740-745, 2000) and other potential ligands that trigger Toll receptors to produce Th1-inducing cytokines, such as synthetic Mycobacterial lipoproteins, Mycobacterial protein p19, peptidoglycan, teichoic acid and lipid A.

Other suitable adjuvant include CT (cholera toxin, subunites A and B) and LT (heat labile enterotoxin from E. coli, subunites A and B), heat shock protein family (HSPs), and LLO (listeriolysin O; WO 01/72329).

Where the immunostimulatory agent is a protein, the agent may be administered either as a protein or as a polynucleotide encoding the protein.

Other suitable delivery systems include microspheres wherein the antigenic material is incorporated into or conjugated to biodegradable polymers/microspheres so that the antigenic material can be mixed with a suitable pharmaceutical carrier and used as a vaccine. The term “microspheres” is generally employed to describe colloidal particles which are substantially spherical and have a diameter in the range 10 nm to 2 mm. Microspheres made from a very wide range of natural and synthetic polymers have found use in a variety of biomedical applications. This delivery system is especially advantageous for proteins having short half-lives in vivo requiring multiple treatments to provide efficacy, or being unstable in biological fluids or not fully absorbed from the gastrointestinal tract because of their relatively high molecular weights. Several polymers have been described as a matrix for protein release. Suitable polymers include gelatin, collagen, alginates, dextran. Preferred delivery systems include biodegradable poly(DL-lactic acid) (PLA), poly(lactide-co-glycolide) (PLG), poly(glycolic acid) (PGA), poly(ε-caprolactone) (PCL), and copolymers poly(DL-lactc-co-glycolic acid) (PLGA). Other preferred systems include heterogeneous hydrogels such as poly(ether ester) multiblock copolymers, containing repeating blocks based on hydrophilic poly-(ethylene glycol) (PEG) and hydrophobic poly(butylene terephtalate) (PBT), or poly(ehtykene glycol)-terephtalate/poly(-butylene terephtalate) (PEGT/PBT) (Sohier et al. Eur. J. Pharm and Biopharm, 2003, 55, 221-228). Systems are preferred which provide a sustained release for 1 to 3 months such as PLGA, PLA and PEGT/PBT.

It is possible for the immunogenic or vaccine composition to be administered on a once off basis or, preferably, to be administered repeatedly, as many times as necessary, for example, between 1 and 7 times, preferably between 1 and 4 times, at intervals between about 1 day and about 18 months, preferably one month. This may be optionally followed by dosing at regular intervals of between 1 and 12 months for a period up to the remainder of the patient's life. In a preferred embodiment the patient receives the antigen in different forms in a “prime boost” regime. Thus for example the antigen, the fusion protein, is first administered as a protein adjuvant base formulation and then subsequently administered as a DNA based vaccine. This administration mode is preferred. The preferred adjuvant is a combination of a CpG-containing oligonucleotide and a saponin derivative, particularly the combination of CpG and QS21 as disclosed in WO 00/09159 and in WO 00/62800. The uptake of naked DNA may be increased by coating the DNA onto biodegradable beads, which are efficiently transported into the cells. Alternatively the DNA can be delivered via a particle bombardment approach, for example, gas-driven particle acceleration with devices such as those manufactured by Powderject Pharmaceuticals PLC (Oxford, UK) and Powderject Vaccines Inc. (Madison, Wis.) as taught herein. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In another preferred embodiment, the DNA based vaccine will be administered first, followed by the protein adjuvant base formulation. Still another embodiment will concern the delivery of the DNA construct by means of specialised delivery vectors, preferably by the means of viral system, most preferably by the means of adenoviral-based systems. Other suitable viral-based systems of DNA delivery include retroviral, lentiviral, adeno-associated viral, herpes viral and vaccinia-viral based systems.

In another preferred embodiment, the protein adjuvant base formulation and DNA based vaccine may be co-administered at adjacent or overlapping sites. Dependent upon the nature of the DNA vaccine formulation, this can be achieved by mixing the DNA and protein adjuvant formulations prior to administration or by simultaneously administration of the DNA and protein adjuvant formulation.

The treatment regime will be significantly varied depending upon the size and species of patient concerned, the amount of nucleic acid vaccine and/or protein composition administered, the route of administration, the potency and dose of any adjuvant compounds used and other factors which would be apparent to a skilled medical practitioner.

Within further aspects, the present invention provides methods for stimulating an immune response in a patient, preferably a T cell response in a human patient, comprising administering a pharmaceutical composition described herein. The patient may be afflicted with lung or colon cancer or colorectal cancer or breast cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with, for example, sarcoma, prostate, ovarian, bladder, lung, colon, colorectal or breast cancer, in which case the methods provide treatment for the disease, or patient considered at risk for such a disease may be treated prophylactically.

The present invention further provides, within other aspects, methods for removing tumour cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4+ and/or CD8+ T cells isolated from a patient with one or more of: (i) a polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Definitions

Also provided by the invention are methods for the analysis of character sequences or strings, particularly genetic sequences or encoded protein sequences. Preferred methods of sequence analysis include, for example, methods of sequence homology analysis, such as identity and similarity analysis, DNA, RNA and protein structure analysis, sequence assembly, cladistic analysis, sequence motif analysis, open reading frame determination, nucleic acid base calling, codon usage analysis, nucleic acid base trimming, and sequencing chromatogram peak analysis.

A computer based method is provided for performing homology identification. This method comprises the steps of: providing a first polynucleotide sequence comprising the sequence of a polynucleotide of the invention in a computer readable medium; and comparing said first polynucleotide sequence to at least one second polynucleotide or polypeptide sequence to identify homology. A computer based method is also provided for performing homology identification, said method comprising the steps of: providing a first polypeptide sequence comprising the sequence of a polypeptide of the invention in a computer readable medium; and comparing said first polypeptide sequence to at least one second polynucleotide or polypeptide sequence to identify homology.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Infommatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GAP program in the GCG program package (Devereux, J., et al., Nucleic Acids Research 12(1): 387 (1984)), BLASTP, BLASTN (Altschul, S. F. et al., J. Molec. Biol. 215: 403-410 (1990), and FASTA (Pearson and Lipman Proc. Natl. Acad. Sci. USA 85; 2444-2448 (1988). The BLAST family of programs is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following:

Algorithm: Needleman and Wunsch, J. Mol. Biol. 48: 443-453 (1970)

Comparison matrix: BLOSSUM62 from Henikoff and Henikoff, Proc. Natl. Acad. Sci. USA. 89:10915-10919 (1992)

Gap Penalty: 8

Gap Length Penalty: 2

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm: Needleman and Wunsch, J. Mol. Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 50, 60, 70, 80, 85, 90, 95, 97 or 100% identity to any of the reference sequences of SEQ ID NO:9 to SEQ ID NO:16, wherein said polynucleotide sequence may be identical to any the reference sequences of SEQ ID NO:9 to SEQ ID NO:16 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in any of SEQ ID NO:9 to SEQ ID NO:16 by the integer defining the percent identity divided by 100 and then subtracting that product from said total number of nucleotides in any of SEQ ID NO:9 to SEQ ID NO:16, or:
nₙ≦xₙ−(xₙ·y)
wherein nₙ is the number of nucleotide alterations, xₙ is the total number of nucleotides in any of SEQ ID NO:9 to SEQ ID NO:16, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of polynucleotide sequences encoding the polypeptides of any of SEQ ID NO:1 to SEQ ID NO:8 may create nonsense, missense or frameshift mutations in this coding sequence and thereby alter the polypeptide encoded by the polynucleotide following such alterations.

By way of example, a polynucleotide sequence of the present invention may be identical to any of the reference sequences of SEQ ID NO:9 to SEQ ID NO:16, that is it may be 100% identical, or it may include up to a certain integer number of nucleic acid alterations as compared to the reference sequence such that the percent identity is less than 100% identity. Such alterations are selected from the group consisting of at least one nucleic acid deletion, substitution, including transition and transversion, or insertion, and wherein said alterations may occur at the 5′ or 3′ terminal positions of the reference polynucleotide sequence or anywhere between those terminal positions, interspersed either individually among the nucleic acids in the reference sequence or in one or more contiguous groups within the reference sequence. The number of nucleic acid alterations for a given percent identity is determined by multiplying the total number of nucleic acids in any of SEQ ID NO:9 to SEQ ID NO:16 by the integer defining the percent identity divided by 100 and then subtracting that product from said total number of nucleic acids in any of SEQ ID NO:9 to SEQ ID NO:16, or
nₙ≦xₙ−(xₙ·y),
wherein nₙ is the number of nucleic acid alterations, xₙ is the total number of nucleic acids in any of SEQ ID NO:9 to SEQ ID NO:16, y is, for instance 0.70 for 70%, 0.80 for 80%, 0.85 for 85% etc., · is the symbol for the multiplication operator, and wherein any non-integer product of xₙ and y is rounded down to the nearest integer prior to subtracting it from xₙ.
(2) Polypeptide embodiments further include an isolated polypeptide comprising a polypeptide having at least a 50, 60, 70, 80, 85, 90, 95, 97 or 100% identity to the polypeptide reference sequence of any of SEQ ID NO:1 to SEQ ID NO:8, wherein said polypeptide sequence may be identical to any of the reference sequence of SEQ ID NO:1 to SEQ ID NO:8 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in any of SEQ ID NO:1 to SEQ ID NO:8 by the integer defining the percent identity divided by 100 and then subtracting that product from said total number of amino acids in any of SEQ ID NO:1 to SEQ ID NO:8, or:
nₐ≦xₐ−(xₐ·y),
wherein nₐ is the number of amino acid alterations, xₐ is the total number of amino acids in SEQ ID NO:2, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

By way of example, a polypeptide sequence of the present invention may be identical to the reference sequence of any of SEQ ID NO:1 to SEQ ID NO:8, that is it may be 100% identical, or it may include up to a certain integer number of amino acid alterations as compared to the reference sequence such that the percent identity is less than 100% identity. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in any of SEQ ID NO:1 to SEQ ID NO:8 by the integer defining the percent identity divided by 100 and then subtracting that product from said total number of amino acids in any of SEQ ID NO:1 to SEQ ID NO:8, or:
nₐ≦xₐ−(xₐ·y)
wherein nₐ is the number of amino acid alterations, xₐ is the total number of amino acids in any of SEQ ID NO:1 to SEQ ID NO:8, y is, for instance 0.70 for 70%, 0.80 for 80%, 0.85 for 85% etc., and · is the symbol for the multiplication operator, and wherein any non-integer product of xₐ and y is rounded down to the nearest integer prior to subtracting it from xₐ.

### FIGURE LEGENDS

FIG. 1: Sequence information for C-LytA. Each repeat has been defined on the basis of both multiple sequence alignment and secondary structure prediction using the following alignment programs: 1) MatchBox (Depiereux E et al. (1992) Comput Applic Biosci 8:501-9); 2) ClustalW (Thompson J D et al. (1994) Nucl Acid Res 22:4673-80); 3) Block-Maker (Henikoff S et al (1995) Gene 163:gc17-26)

FIG. 2: CPC and native Constructs (SEQ ID NOs. 27-36)

FIG. 3: Schematic structure of CPC-p501 His fusion protein expressed in S. cerevisiae

FIG. 4: Primary structure of CPC-P501 His fusion protein (SEQ ID NO.41)

FIG. 5: Nucleotide sequence of CPC P501 His (pRIT15201) (SEQ ID NO.42)

FIG. 6: Cloning strategy for generation of plasmid pRIT 15201

FIG. 7: Plasmid map of pRIT15201

FIG. 8. Comparative expression of CPC P501 and P501 in S. cerevisiae strain DC5

FIG. 9: Production of CPC-P501S HIS (Y1796) at small scale. FIG. 9A represents the antigen productivity as estimated by SDS-PAGE with silver staining; FIG. 9B represents the antigen productivity as estimated by western blot.

FIG. 10: Purification scheme of CPC-P501-His produced by Y1796.

FIG. 11: Pattern of CPC P501 His purified protein (4-12% Novex Nu-Page polyacrylamide precasted gels).

FIG. 12: Native full-length P501S sequence (SEQ ID NO:17)

FIG. 13: Sequence of the CPC-P501S expression cassette of JNW735 (SEQ ID NO:18)

FIG. 14: Two codon optimised P501S sequences (SEQ ID NO:19-20)

FIG. 15: Re-engineered codon optimised sequence 19 (SEQ ID NO:21)

FIG. 16: Re-engineered codon optimised sequence 20 (SEQ ID NO:22)

FIG. 17: The starting sequence for the optimisation of CPC (SEQ ID NO:23)

FIG. 18: Representative codon optimised CPC sequences (SEQ ID NO:24-25)

FIG. 19: Engineered CPC codon optimised sequence (SEQ ID NO:26)

FIG. 20: P501S CPC fusion candidate constructs and sequences (SEQ ID NOs. 3740 & 4548)

FIG. 21: Western blot analysis of CHO cells following transient transfection with P501S (JNW680), CPC-P501S (JNW735) and empty vector control.

FIG. 22: Anti-P501S antibody responses following immunisation at day 0, 21 & 42 with pVAC-P501S (JNW680, mice B1-9) or Empty vector (pVAC, mice A1-6). A pre-bleed was taken at day −1. Subsequently bleeds were taken at day 28 and day 49 (mice A1-3, B1-3) and day 56 (mice A4-6, B4-9). All sera was tested at 1/100 dilution. The results for the pVAC immunised mice were averaged. The results for the individual pVAC-P501S immunised mice are shown. As a positive control, sera from Adeno-P501S immunised mice (Corixa Corp, diluted 1/100) is included.

FIG. 23: Peptide library screen using C57BL/6 mice immunised at day 0, 21, 42, and 70 with pVAC-P501S (JNW680). All peptides were used at a final concentration of 50 μg/ml. Peptides 1-50 are overlapping 15-20mers obtained from Corixa. Peptides 51-70 are predicted 8-9mer Kb and Db epitopes and were ordered from Mimotopes (UK). Samples 71-72 and 73-78 are DMSO controls and no peptide controls respectively. Graph A shows the IFN-γ responses whilst Graph B shows the IL-2 responses. Peptides selected for use in subsequent immunoassays are shown in black.

FIG. 24: Cellular responses by ELISPOT at day 77 following PMID immunisation at day 0, 21, 42, and 70 with pVAC-P501S (JNW680, B6-9) and pVAC empty (A4-6). Peptide 18, 22 & 48 were used at 50 μg/ml. CPC-P501S protein was used at 20 μg/ml. Graph A shows the IFN-γ responses whilst Graph B shows the IL-2 responses.

FIG. 25: Comparison of P501S and CPC-P501S. Cellular responses were measured by IL-2 ELISPOT using peptide 22 (10 μg/ml) at day 28. Mice were immunised by PMID at day 0 and 21 with pVAC empty (control), pVAC-P501S (JNW680) and CPC-P501S (JNW735).

FIG. 26: Immune response (lymphoproliferation on spleen cells) following protein immunisation with CPC-P501S.

FIG. 27: Evaluation of the immune response to different CPC-P501S constructs. Cellular responses were measured by IL-2 ELISPOT at day 28. Mice were immunised by PMID at day 0 and 21 with p7313-ie empty (control), JNW735 and CPC-P501S constructs (JNW770, 771 and 773)

FIG. 28: MUC-1 CPC sequences (SEQ ID NOs. 49 & 50)

FIG. 29: ss-CPC-MUC-1 sequences (SEQ ID NOs. 51 & 52)

The invention will be further described by reference to the following examples:

### EXAMPLE I

### Preparation of the Recombinant Yeast Strain Y1796 Expressing P501 Fusion Protein Containing a C-LytA-P2-C-LytA (CPC) as fusion partner

1.—Protein Design

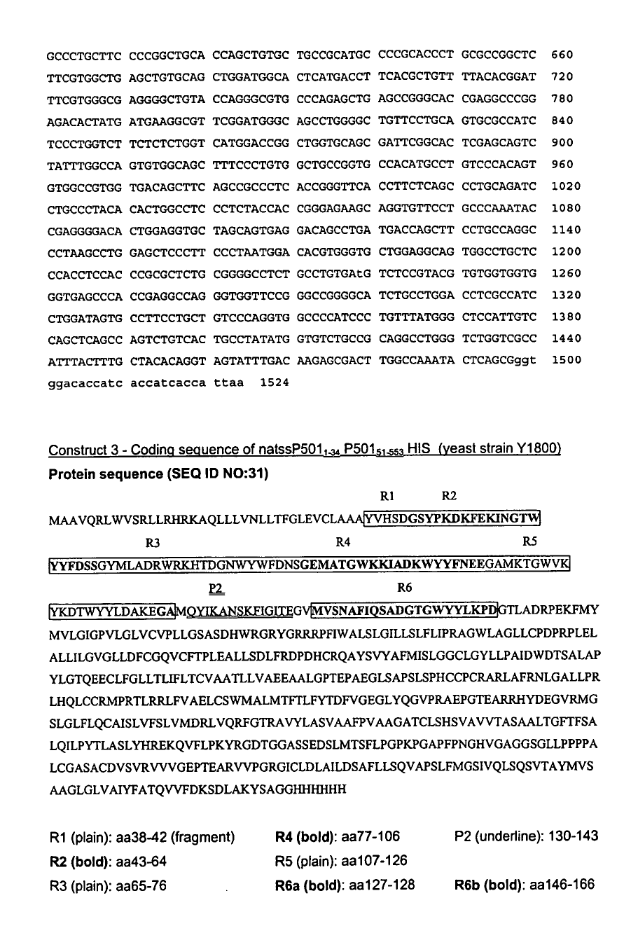

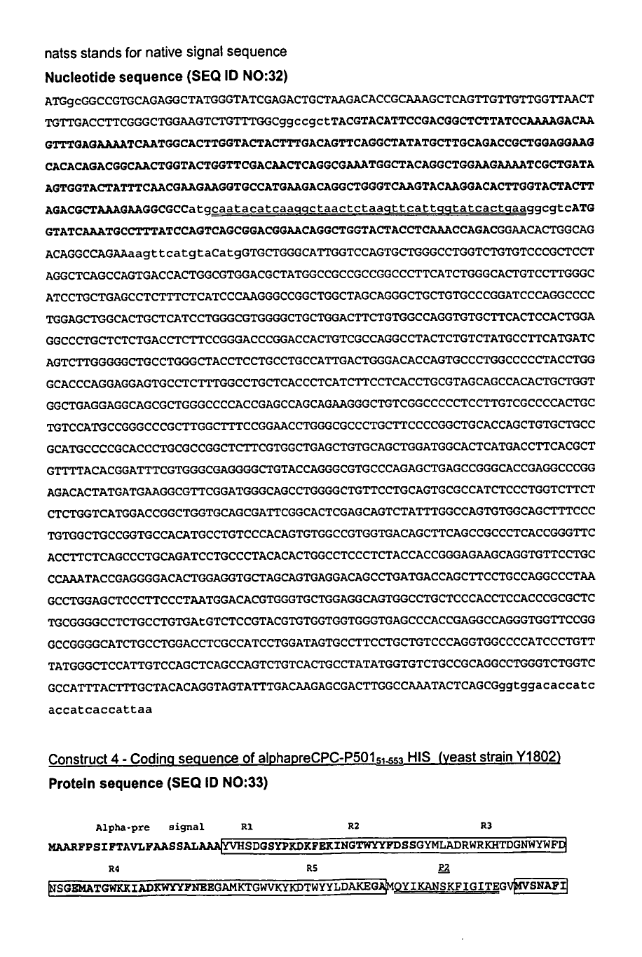

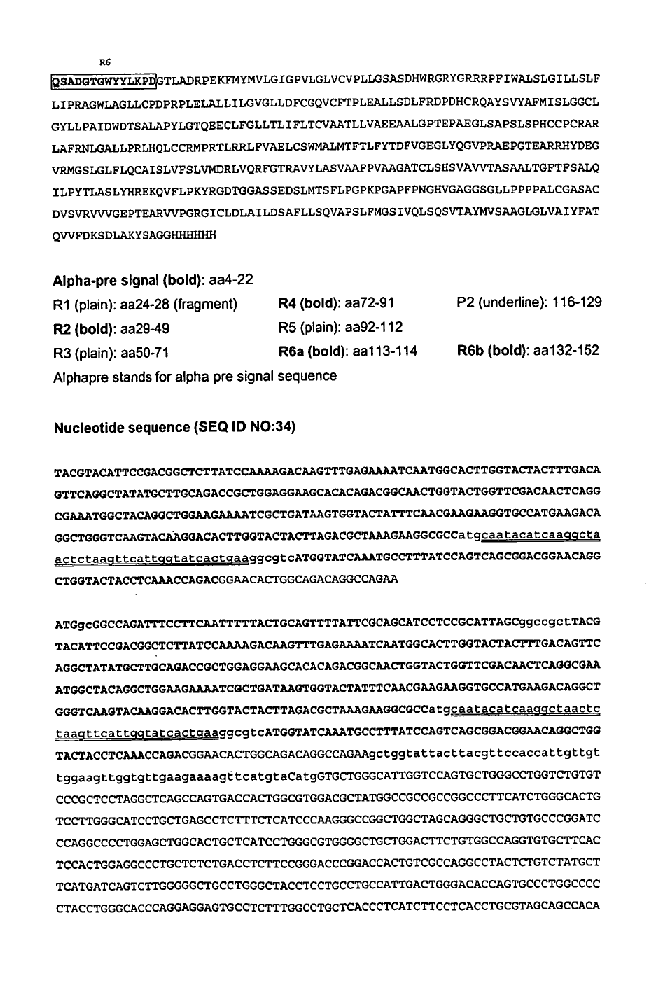

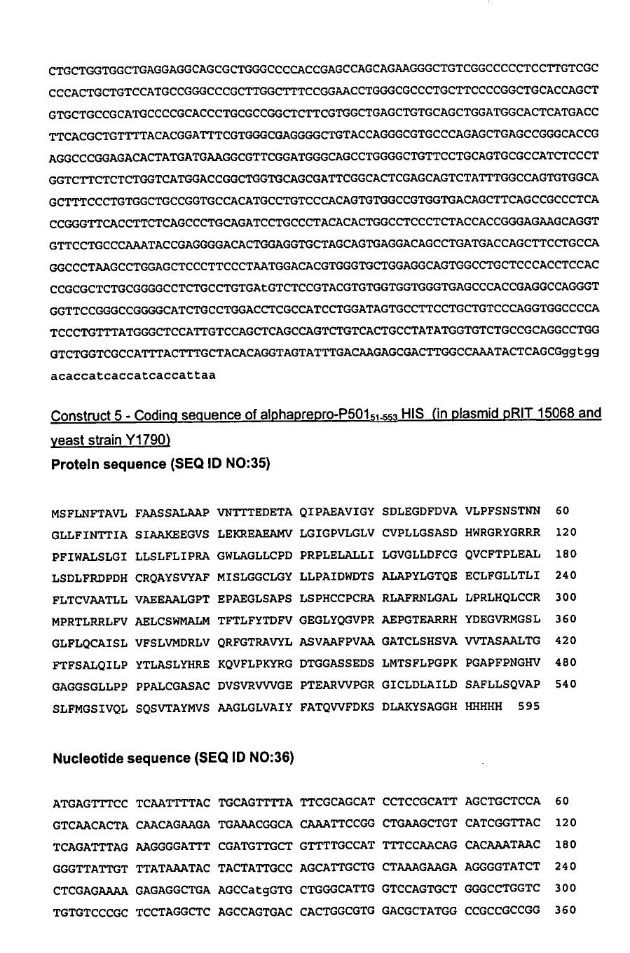

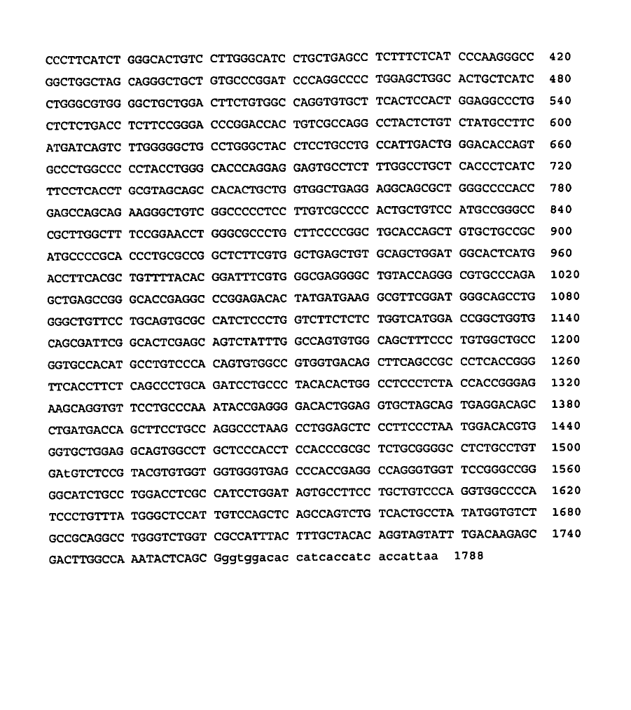

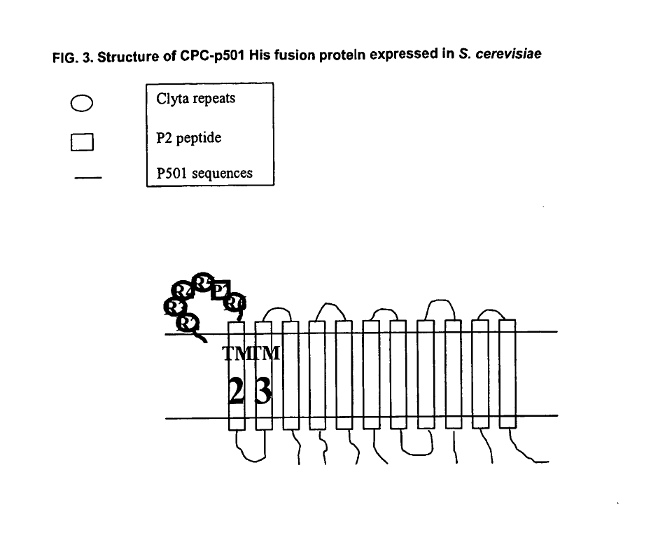

The structure of the fusion protein C-P2-C-p501 (alternatively named CPC-P501) to be expressed in S. cerevisiae is depicted in FIG. 3. This fusion contains the C-terminal region of gene LytA (residues 187 to 306), in which the P2 fragment of tetanus toxin (residues 830-843) has been inserted. The P2 fragment is placed between the residues 277 and 278 of C-Lyt-A. The C-lytA fragment containing the P2 insertion is followed by P501 (residues amino acid 51 to 553) and by the His tail.

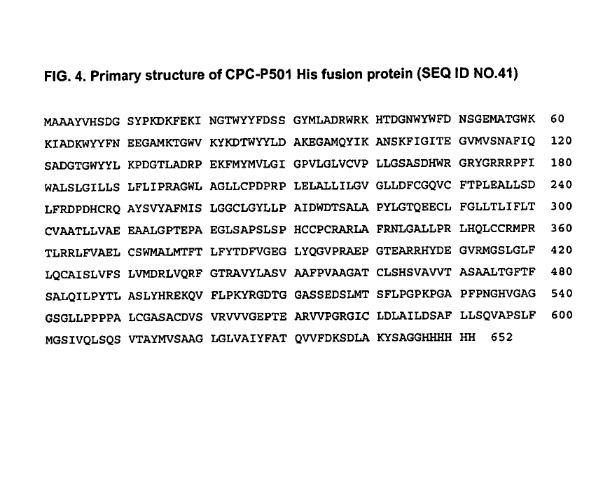

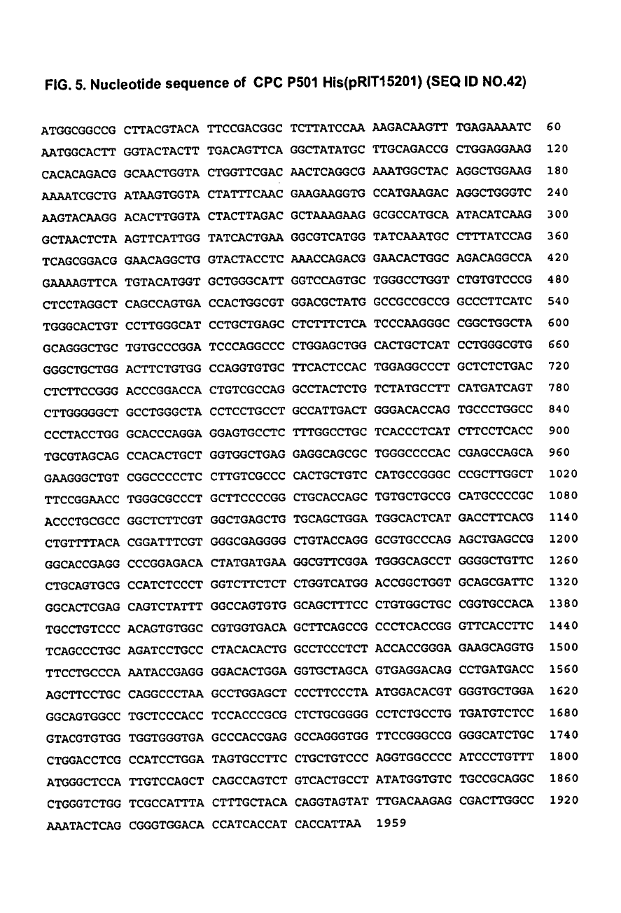

The primary structure of the resulting fusion protein has the sequence described in FIG. 4 and the coding sequence corresponding to the above protein design is in FIG. 5.

2.—Cloning Strategy for the Generation of a Yeast Plasmid Expressing CPC-P501 (51-553)-His Fusion Protein

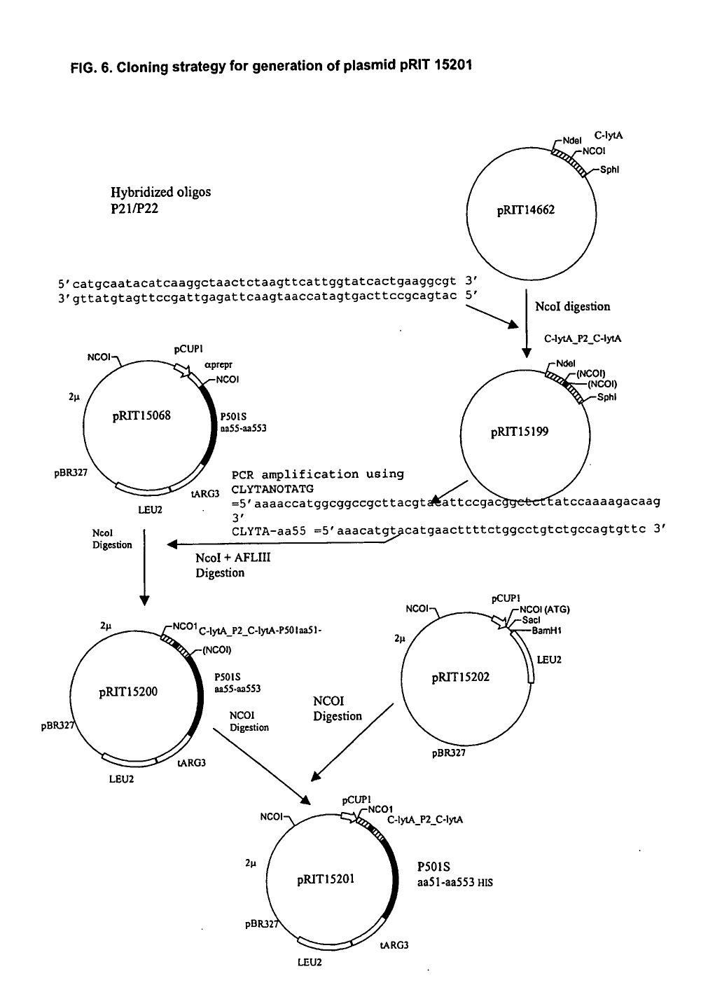

The starting material is the yeast vector pRIT15068 (UK patent application 0015619.0).
This vector contains the yeast Cup1 promoter, the yeast alpha prepro signal coding sequence and the coding sequence corresponding to residues 55 to 553 of P501S followed by His tail.
The cloning strategy outlined in FIG. 6 include the following steps:
   a) The first step is the insertion of P2 sequence (codon-optimised for yeast expression) in frame, inside the C-lytA coding sequence. The C-lytA coding sequence is harbored by plasmid pRIT 14662 (PCT/EP99100660). The insertion is done using an adaptor formed by two complementary oligonucleotides named P21 and P22 into the plasmid pRIT 14662 previously open by NcoI

The sequence of P21 and P22 is:

P21 5′ catgcaatacatcaaggctaactctaagttcattggtatcactgaaggcgt 3′

P22 3′ gttatgtagttccgattgagattcaagtaaccatagtgacttccgcagtac 5′

After ligation and transformation of E. coli and transformant characterization, the plasmid named pRIT15199 is obtained.

b) The second step is the preparation of C-lytA-P2-C-lytA DNA fragment by PCR amplification. The amplification is performed using pRIT15199 as template and the oligonucleotides named C-LytANOTATG and C-LytA-aa55. The sequence of both oligonucleotides being:

C-LytANOTATG

=5′aaaaccatggcggccgcttacgtacattccgacggctcttatccaaaagacaag 3′ C-LytA-aa55=5′aaacatgtacatgaacttttctggcctgtctgccagtgttc 3′

The amplified fragment is treated with the restriction enzymes NcoI and Afl III to generate the respective cohesive ends.

c) The next step is the ligation of the above fragment with vector pRIT15068 (largest fragment obtained after NcoI treatment) to generate the complete fusion protein coding sequence. After ligation and E. coli transformation the plasmid named pRIT15200 is obtained. In this plasmid the remaining unique NcoI site contains the ATG coding for the start codon.

d) In the next step a NcoI fragment containing the CUP1 promoter and a portion of 2μ plasmid sequences is prepared from plasmid PRIT 15202. Plasmid pRIT 15202 is a yeast 2μ derivative containing the CUP1 promoter with an NcoI site at ATG (ATG sequence: AAACC ATG)

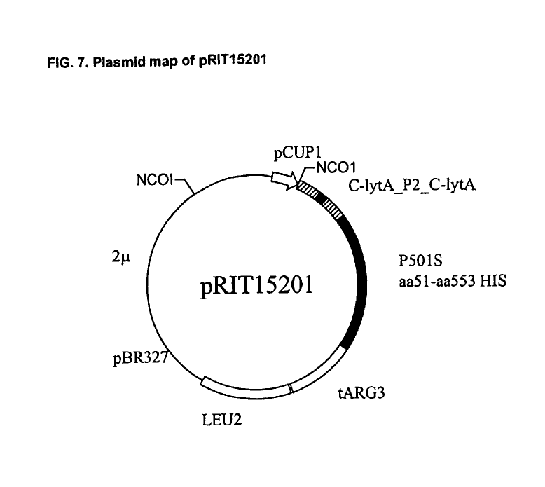

e) The NcoI fragment isolated from PRIT 15202 is ligated to pRIT15200, previously open with NcoI, in the righ orientation, in such a way the pCUP1 promoter is at the 5′ side of the coding sequence. This results in the generation of a final expression plasmid named pRIT15201 (see FIG. 7).

3.—Preparation of the Recombinant Yeast Strain Y1796 (RIX4440)

The plasmid pRIT 15201 is used to transform the S. cerevisiae strain DC5 (ATCC 20820). After selection and characterisation of the yeast transformants containing the plasmid pRIT 15201 a recombinant yeast strain named Y1796 expressing CPC-P501-His fusion protein is obtained. The protein after reduction and carboxyamidation, is isolated and purified by affinity chromatography (IMAC) followed by anion exchange chromatography (Q Sepharose FF).

### EXAMPLE II

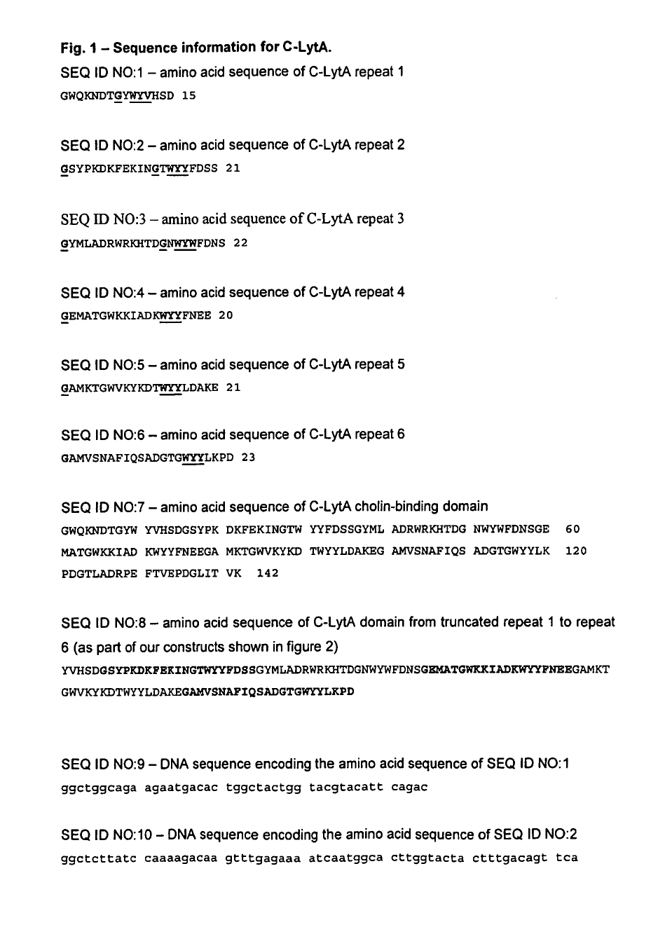

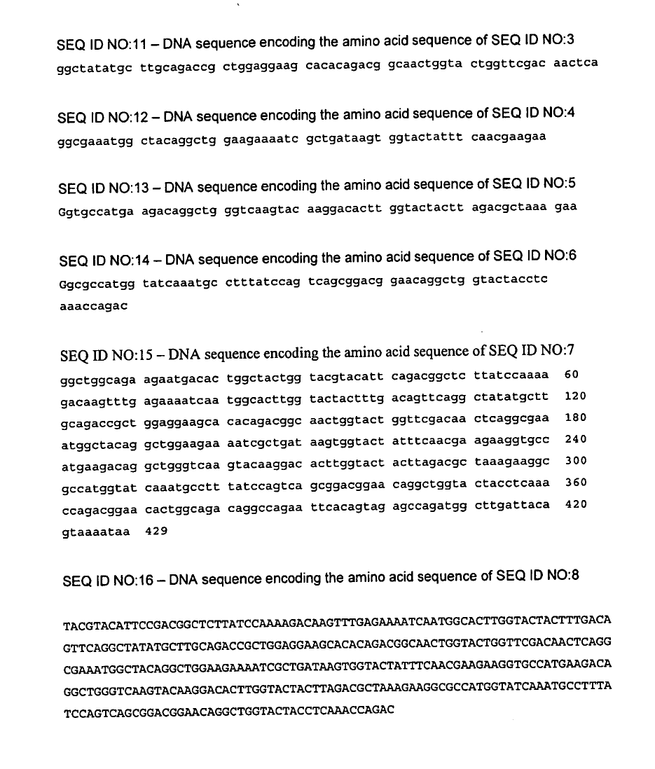

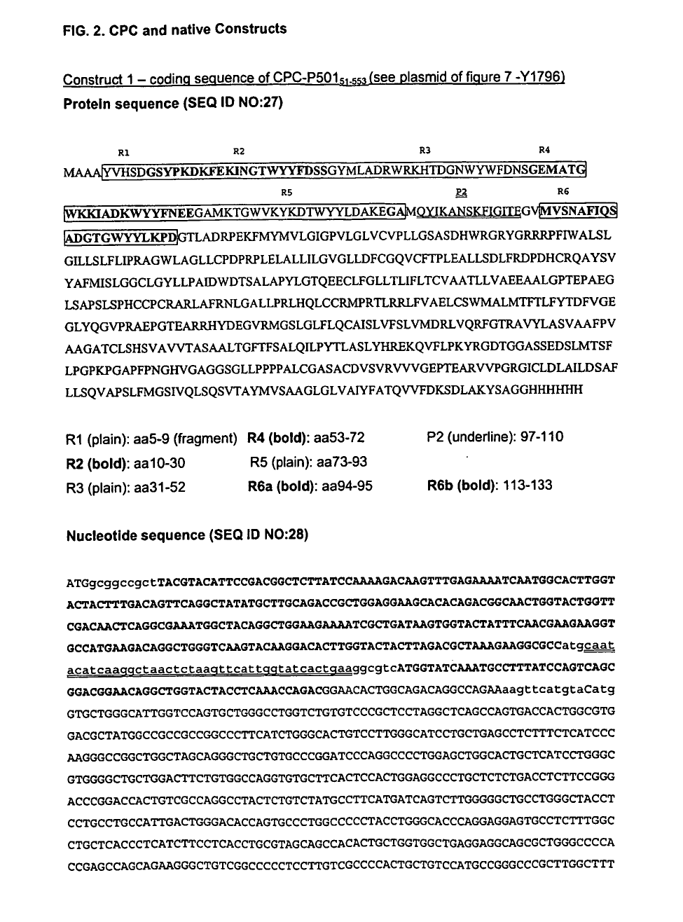

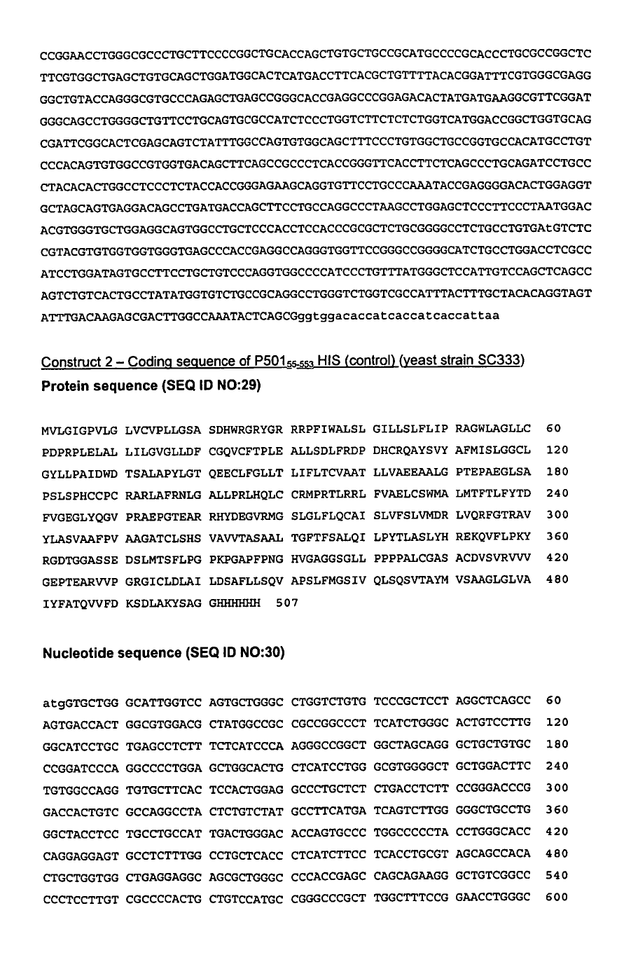

In analogous fashion proteins constructs as depicted in FIG. 2 may be expressed utilising the corresponding DNA sequences shown therein. In particular, yeast strain SC333 (construct 2) corresponds to Y1796 strain but expressing P501₅₅₋₅₅₃ devoid of the CPC fusion partner. Yeast strain Y1800 (construct 3) corresponds to Y1796 strain but additionally comprises the native sequence signal for P501S (aa1-aa34), while yeast strain Y1802 (construct 4) comprises the alpha pre signal sequence upstream CPC-P501S sequence. Yeast strain Y1790 (construct 5) is expressing a P501S construct devoid of CPC and having the alpha prepro signal sequence.

### EXAMPLE III

### Preparation of Purified CPC-P501

1.—Production of CPC-P501S HIS (Y1796) at Small Scale

For Y1796, in minimal medium supplemented with histidine, expression is induced in log phase by addition of CuSO4 ranging from 100 to 500 μM, and culture is maintained at 30°. Cells are harvested after 8 or 24H induction. Copper is added just before use and not mixed with medium in advance.

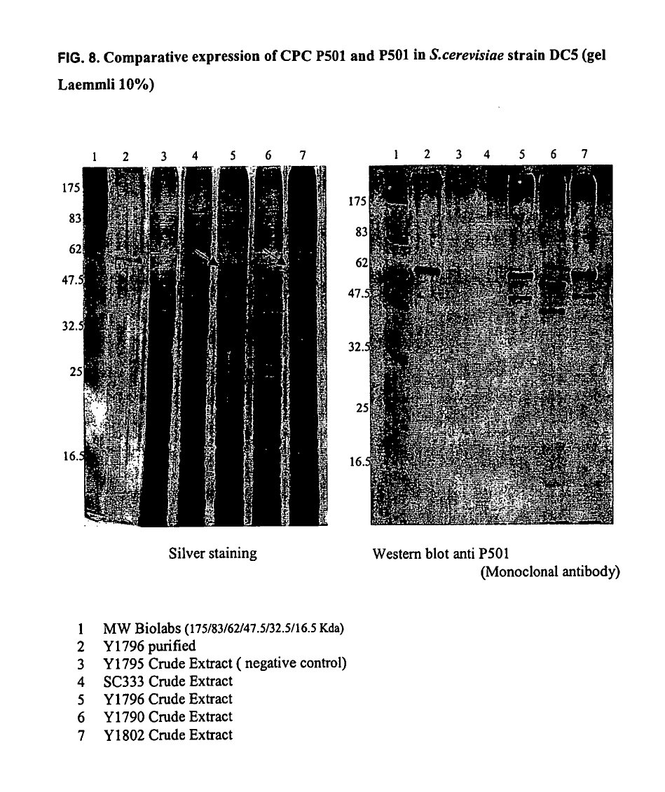

For SDS PAGE analysis, yeast cells extraction is performed in citrate phosphate buffer pH4.0+130 mM NaCl. Extraction is performed with glass beads for small cell quantity and with French press for higher cells quantity, and then mixed with sample buffer and SDS-PAGE analysed. Results of comparative analysis on SDS PAGE of the different constructs are depicted in FIG. 8 and summariosed in Table 2 below.

As shown in Table 1 below, the level of expression of the culture is much higher for Y1796 strain as compared to the expression level of parent strain SC333, a strain expressing the corresponding P501S-His devoid of CPC partner. Likewise, the presence of a signal sequence (alpha pre) does not affect the results discussed above: the level of expression of the culture is much higher for Y1802 strain as compared to the expression level of corresponding strain Y1790, a strain expressing the corresponding P501S-His devoid of CPC partner.

TABLE 2

| Recombinant | | | Signal | Fusion | P501 aa | Expression |
|---|---|---|---|---|---|---|
| Strain | Plasmid | Promotor | sequence | Partner | sequences | level |
| SC333 | Ma333 | CUP 1 | — | — | 55-553-His | ⊖ND |
| Y1796 | pRIT 15201 | CUP 1 | — | CPC | 51-553-His | +++ |
| Y1802 | pRIT 15219 | CUP 1 | α pre | CPC | 51-553-His | ++++ |
| Y1790 | pRIT 15068 | CUP 1 | α prepro | — | 55-553-His | + |

CPC = clyta P2 clyta
ND = not detectable, even in western blot
+ = detectable in western blot
+++/++++ = detectable in western blot and visible in silver stained gels

2.—Fermentation of Y1796 (RIX4440) at Larger Scale

100 μl of the working seed are spread on solid medium and grown for approximately 24 h at 30° C. This solid pre-culture is then used to inoculate a liquid pre-culture in shake flasks.

This liquid pre-culture is grown for 20 h at 30° C. and transferred into a 20 L fermenter. The fed-batch fermentation includes a growth phase of about 44 h and an induction phase of about 22 h.

The carbon source (glucose) was supplemented to the culture by a continuous feeding. The residual glucose concentration was maintained very low (≦50 mg/L) in order to minimise the ethanol production by fermentation. This was realised by limiting the development of the micro-organism by limited glucose feed rate.

At the end of the growth phase, CUP1 promoter is induced by adding CuSO4 in order to produce the antigen.

The absence of contaminations was checked by inoculating 10⁶ cells into standard TSB and THI vials supplemented with nystatine and incubated respectively for 14 days at 20-25° C. and at 30-35° C. No growth was observed as expected.

3.—Antigen Characterisation and Productivity

Cell homogenates were prepared by French pressing of fermentation samples harvested at different times during the induction phase and analysed by SDS-PAGE and Western Blot. It was shown that the major part of the protein of interest was located in the insoluble fraction obtained from the cell homogenate after centrifugation. The SDS-PAGE and Western Blot analyses shown in the Figures below were realised on the pellets obtained after centrifugation of these cell homogenates.

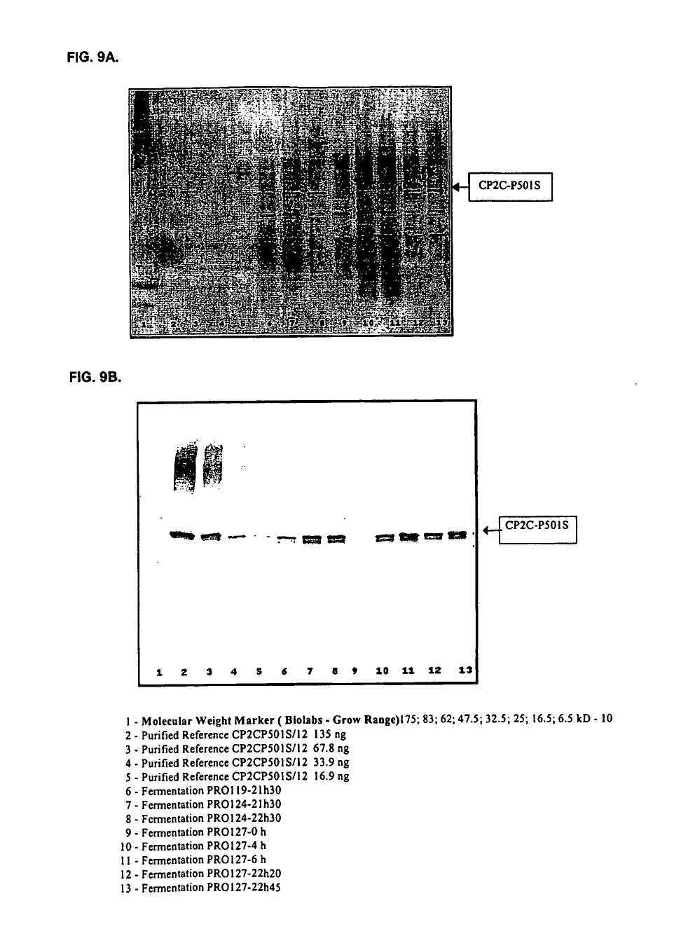

FIGS. 8 A and B show a kinetics of the antigen production during the induction phase for culture PRO127. It appears that no antigen expression occurred during the growth phase. The specific antigen productivity seems to increase from the beginning of the induction phase up to 6 h and then remained quite stable up to the end. But the volumetric productivity increased by a factor 1.5 to 2 due to biomass accumulation observed during the same period of time. The antigen productivity was estimated at about 500 mg per litre of fermentation broth by comparing purified reference of the antigen and crude extracts on SDS-PAGE with silver staining (FIG. 9A) and WB analyses using an anti-P501S antibody (a murine ascite directed against P501S aa439-aa459 used at a dilution of 1/1000) (FIG. 9B).

### EXAMPLE IV

### Purification of CPC-P501 (51-553)-His Fusion Protein Produced by Y1796

After the cell breakage, the protein is associated with the pellet fraction. A carbamido-methylation of the molecule has been introduced in the process in order to cope with the oxidative aggregation of the molecule with itself or with host cell protein contaminants through covalent bridging with disulphide bonds. The use of detergents has also been required to manage the hydrophobic character of this protein (12 trans-membrane domains predicted).

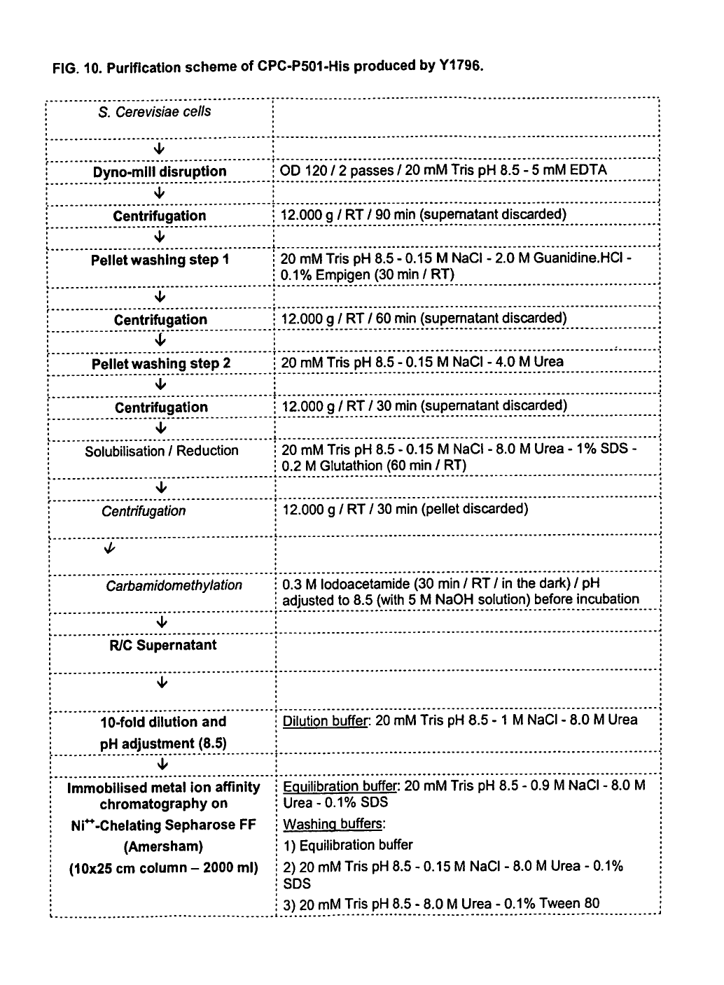

The purification protocol, developed for the scale of 1 L of culture OD (optical density) 120, is described in FIG. 10. All the operations are performed at room temperature (RT).

According to DOC TCA BCA protein assay, the global purification yield is 30-70 mg of purified antigen/L of culture OD 120. The yield is linked to the level of expression of the culture and is higher as compared to the purification yield of parent strain expressing unfused P501S-His.

The protein assay is performed as followed: proteins are first precipitated using TCA (trichloroacetic acid) in the presence of DOC (deoxycholate) then dissolved in a alcaline medium in the presence of SDS. The proteins then react with BCA (bicinchoninic acid) (Pierce) to form a soluble purple complex presenting a high adsorbance at 562 nm, which is proportional to the amount of proteins present in the sample.

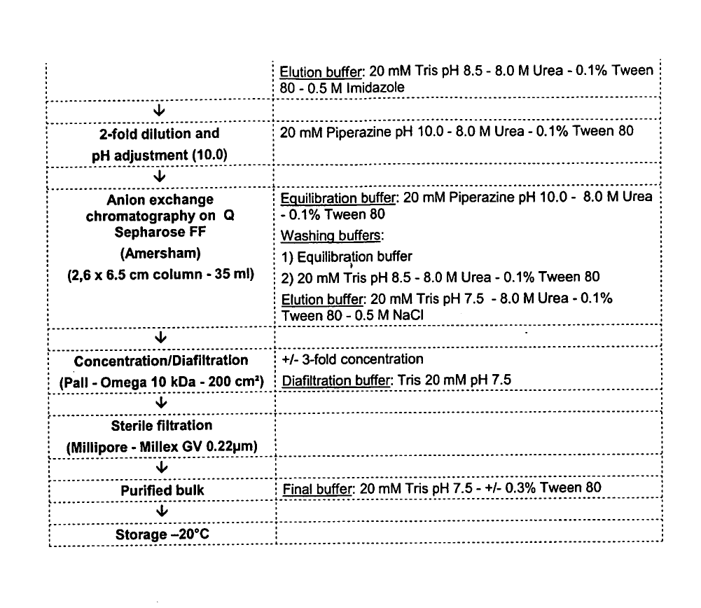

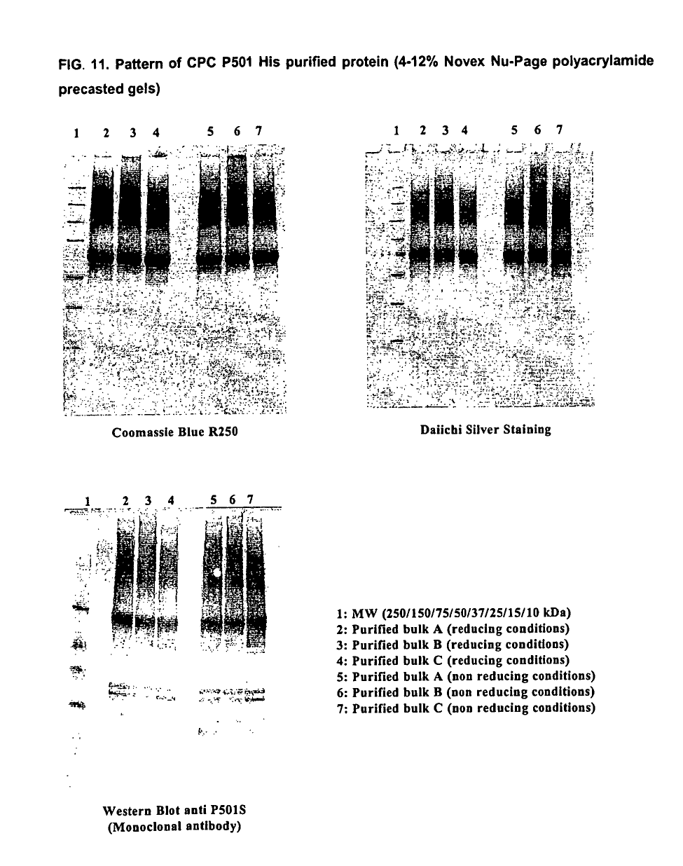

SDS-PAGE analysis of 3 purified bulks (FIG. 11) shows no difference in reducing and non reducing conditions (cf. lanes 2, 3 and 4 versus lanes 5, 6 and 7). The pattern consists of a major band at 70 kDa, a smear of higher MW and faint degradation bands. All the bands are detected by a specific anti P501S monoclonal antibody.

### EXAMPLE V

### Vaccine Preparation Using CPC-P501S His Protein

The protein of Example 3 or 4 can be formulated into a vaccine containing QS21 and 3D-MPL in an oil in water emulsion.

1.—Vaccine Preparation:

The antigen produced as shown in Example 1 to 3 a C-LytA-P2-P501S His. As an adjuvant, the formulation comprises a mixture of 3 de-O-acylated monophosphoryl lipid A (3D-MPL) and QS21 in an oil/water emulsion. The adjuvant system SBAS2 has been previously described WO 95/17210.

3D-MPL: is an immunostimulant derived from the lipopolysaccharide (LPS) of the Gram-negative bacterium Salmonella minnesota. MPL has been deacylated and is lacking a phosphate group on the lipid A moiety. This chemical treatment dramatically reduces toxicity while preserving the immunostimulant properties (Ribi, 1986). Ribi Immunochemistry produces and supplies MPL to SB-Biologicals.

Experiments performed at Smith Kline Beecham Biologicals have shown that 3D-MPL combined with various vehicles strongly enhances both the humoral and a TH1 type of cellular immunity.

QS21: is a natural saponin molecule extracted from the bark of the South American tree Quillaja saponaria Molina. A purification technique developed to separate the individual saponins from the crude extracts of the bark, permitted the isolation of the particular saponin, QS21, which is a triterpene glycoside demonstrating stronger adjuvant activity and lower toxicity as compared with the parent component. QS21 has been shown to activate MHC class I restricted CTLs to several subunit Ags, as well as to stimulate Ag specific lymphocytic proliferation (Kensil, 1992). Aquila (formally Cambridge Biotech Corporation) produces and supplies QS21 to SB-Biologicals.

Experiments performed at SmithKline Beecham Biologicals have demonstrated a clear synergistic effect of combinations of MPL and QS21 in the induction of both humoral and TH1 type cellular immune responses.

The oil/water emulsion is composed an organic phase made of of 2 oils (a tocopherol and squalene), and an aqueous phase of PBS containing Tween 80 as emulsifier. The emulsion comprised 5% squalene 5% tocopherol 0.4% Tween 80 and had an average particle size of 180 nm and is known as SB62 (see WO 95/17210).

Experiments performed at SmithKline Beecham Biologicals have proven that the adjunction of this O/W emulsion to 3D-MPUQS21 (SBAS2) further increases the immunostimulant properties of the latter against various subunit antigens.

2.—Preparation of Emulsion SB62 (2 Fold Concentrate):

Tween 80 is dissolved in phosphate buffered saline (PBS) to give a 2% solution in the PBS. To provide 100 ml two fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an M110S microfluidics machine. The resulting oil droplets have a size of approximately 180 nm.

3.—Formulations:

A typical formulation containing 3D-MPL and QS21 in an oil/water emulsion is performed as follows: 20 μg-25 μg C-LytA P2-P501S are diluted in 10 fold concentrated of PBS pH 6.8 and H₂O before consecutive addition of SB62 (50 μl), MPL (20 μg), QS21 (20 μg), optionally comprising CpG oligonucleotide (100 μg) and 1 μg/ml thiomersal as preservative. The amount of each component may vary as necessary. All incubations are carried out at room temperature with agitation.

### EXAMPLE VI

### Codon-Optimised P501S Sequences

1.—Generation of the Control Recombinant Plasmids:

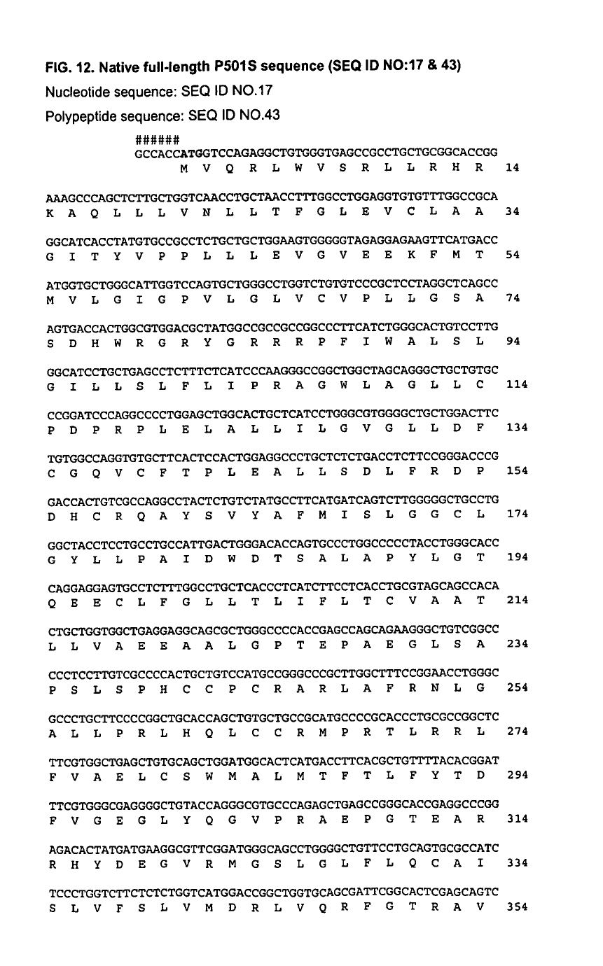

Full-length P501S sequence was cloned into pVAC (Thomsen, Immunology, 1998; 95:51OP105), generating expression plasmid JNW680. SEQ ID NO:17 represents human P501S expression cassette in the plasmid JNW680 and is illustrated in FIG. 12. The protein sequence of SEQ ID NO:17 is shown in single letter format, the start and stop codons being shown in bold. The Kozak sequence is denoted by the hash symbols. The codon usage index of the human P501S sequence (SEQ ID NO:17) is 0.618, as calculated by the SynGene programme.

SynGene Programme

Basically, the codons are assigned using a statistical method to give synthetic gene having a codon frequency closer to that found naturally in highly expressed E. coli and human genes.

SynGene is an updated version of the Visual Basic program called Calcgene, written by R. S. Hale and G Thompson (Protein Expression and Purification Vol. 12 pp. 185-188 (1998). For each amino acid residue in the original sequence, a codon was assigned based on the probability of it appearing in highly expressed E. coli genes. Details of the Calcgene program, which works under Microsoft Windows 3.1, can be obtained from the authors. Because the program applies a statistical method to assign codons to the synthetic gene, not all resulting codons are the most frequently used in the target organism. Rather, the proportion of frequently and infrequently used codons of the target organism is reflected in the synthetic sequence by assigning codons in the correct proportions. However, as there is no hard-and-fast rule assigning a particular codon to a particular position in the sequence, each time it is run the program will produce a different synthetic gene—although each will have the same codon usage pattern and each will encode the same amino acid sequence. If the program is run several times for a given amino acid sequence and a given target organism, several different nucleotide sequences will be produced which may differ in the number, type and position of restriction sites, intron splice signals etc., some of which may be undesirable. The skilled artisan will be able to select an appropriate sequence for use in expression of the polypeptide on the basis of these features.

Furthermore, since the codons are randomly assigned on a statistical basis, it is possible (although perhaps unlikely) that two or more codons which are relatively rarely used in the target organism might be clustered in close proximity. It is believed that such dusters may upset the machinery of translation and result in particularly low expression rates, so the algorithm for choosing the codons in the optimized gene excludes any codons with an RSCU value of less than 0.2 for highly expressed genes in order to prevent any rare codon clusters being fortuitously selected. The distribution of the remaining codons is then allocated according to the frequencies for highly expressed E. coli to give an overall distribution within the synthetic gene that is typical such genes (coefficient=0.85) and also for highly expressed human genes (coefficient=0.50).

Syngene (Peter Ertl, unpublished), an updated version of the Calcgene program, allows exclusion of rare codons to be optional, and is also used to allocate codons according to the codon frequency pattern of highly expressed human genes.

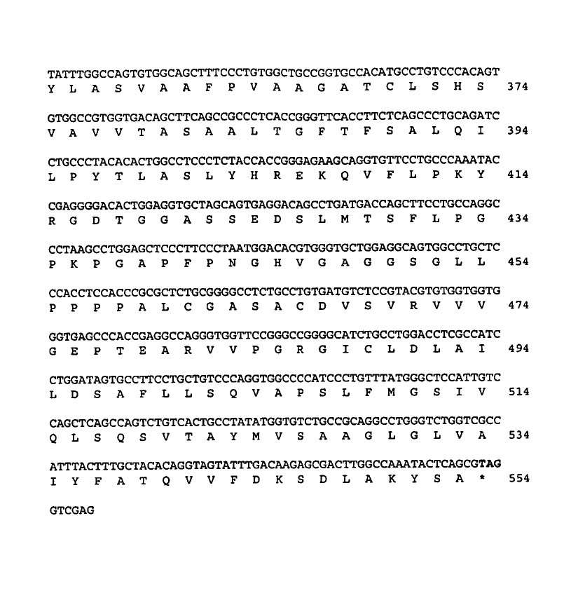

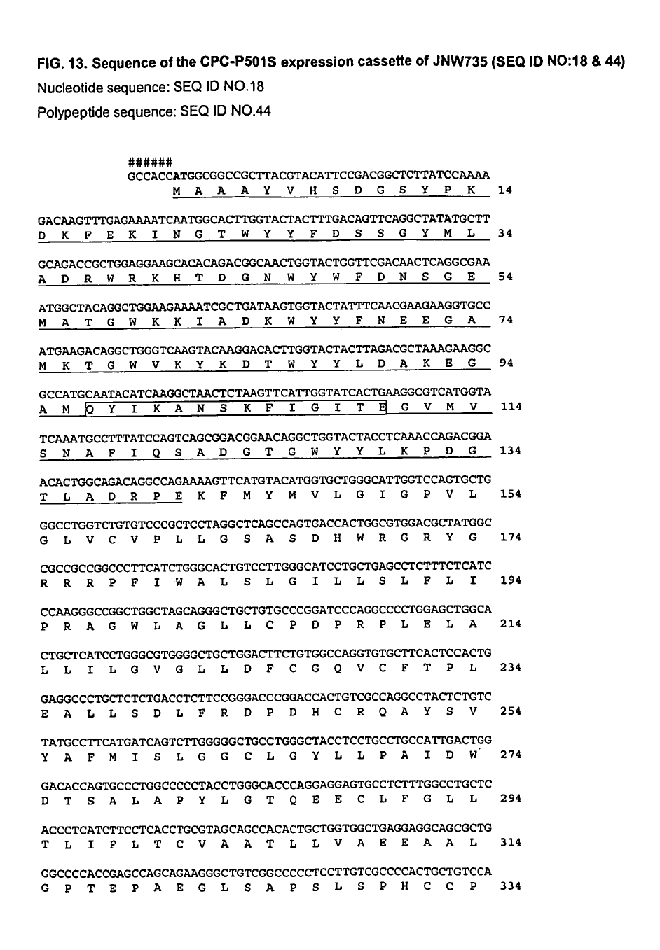

The sequence of the CPC-P501S cassette cloned from the vector pRIT15201 (see FIG. 7) into pVAC, thereby generating plasmid JNW735, is set forth in SEQ ID NO:18 and is illustrated in FIG. 13. This sequence is identical to the pRIT15201 sequence with the exception of the removal of the His tag and the addition of a Kozak sequence (GCCACC) and appropriate restriction enzyme sites. The amino acid sequence of SEQ ID NO:18 is shown in single letter format, the start and stop codons are shown in bold. The boxed residues are the P2 helper epitope of tetanus toxoid. The underlined residues are the Clyta purification tag. The Kozak sequence is denoted by the hash symbols.

2.—Generation of the Recombinant Plasmids with P501S Codon Optimised Sequences:

Although the codon coefficient index (CI) of P501S native sequence is already high (0.618), it is possible increase the CI value further. This will have two potential benefits—to improve the antigen expression and/or immunogenicity and to reduce the possibility for recombination between the P501S vector and genomic sequences.

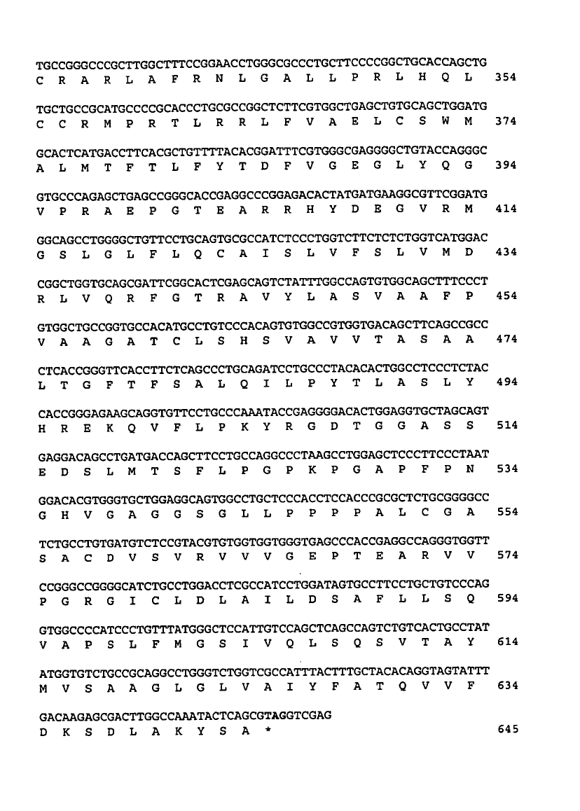

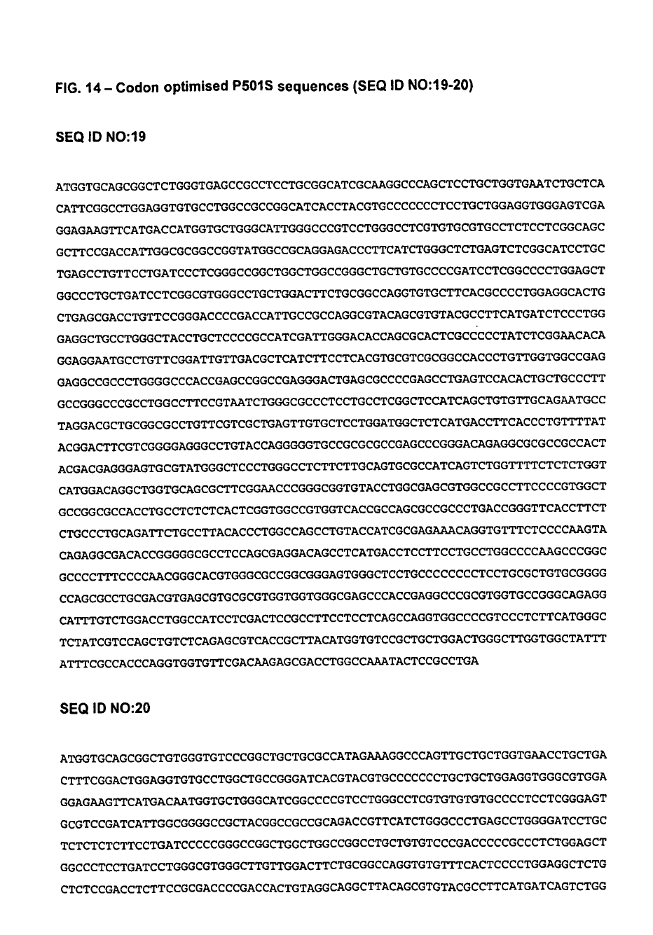

Using the Syngene programme, a selection (SEQ ID NO:19 to SEQ ID NO:20) of codon optimised sequences was obtained (FIG. 14). Table 3 below shows a comparison of the codon coefficient index for the starting P501S sequence and the two representative codon optimised sequences, selected on the basis of a suitable restriction enzyme site profile and a good CI index.

TABLE 3
Comparison of the codon coefficient indices
of two codon optimised P501S genes

| Sequence | Codon coefficient Index (CI) |
|---|---|
| P501S | 0.618 |
| SEQ ID NO: 19 | 0.725 |
| SEQ ID NO: 20 | 0.755 |

3. Further Evaluation of the Codon-Optimised Sequences
Sequence SEQ ID NO:19

Although SEQ ID NO: 19 has a good CI index (0.725), it contains a doublet of rare codons at amino acids position 202 and 203. These codons were manually substituted with more frequent codons by changing the DNA sequence from TTGTTG to CTGCTG. To facilitate cloning and expression, restriction enzyme sites and a Kozak sequence were added. The final engineered sequence (SEQ ID NO:21) is shown in FIG. 15. The Syngene programme was used to fragment this sequence into oligonucleotides with a minimum overlap of 19-20 bases. Therefore, FIG. 15 shows the re-engineered P501S codon optimised SEQ ID NO. 19. Restriction enzyme sites are underlined, Kozak sequence is bolded, re-engineered DNA sequence to remove a rare codon doublet is boxed.

Using a two-step PCR protocol, the overlapping primers generated by the Syngene programme were first assembled using a PCR Assembly protocol (detailed below). The assembly reaction generates a diverse population of fragments. The correct full-length fragment was recovered/amplified using the PCR recovery protocol and the terminal primers. The resulting PCR fragment was excised from an agarose gel, purified, restricted with NheI and XhoI and cloned into pVAC. Positive clones were identified by restriction enzyme analysis and confirmed by double-stranded sequencing. This generates plasmid JNW766, which, due to the error-prone nature of the PCR process, contained a single silent mutation (C to T at position 360 of SEQ ID NO: 21).

1. Assembly Reaction—PCR Conditions, Generic Protocol

Reaction mix (total volume=50 μl):

1× Reaction buffer (Pfx or Proofstart)
1 μl Oligo pool (equal mix of all overlapping oligos)
0.5 mM dNTPs
DNA polymerase (Pfx or Proofstart, 2.5-5U)
+/−1 mM MgSO₄
+/−1× enhancer solution (Pfx enhancer or Proofstart buffer Q)
   1. 94° C. for 120 s (Proofstart only)
   2. 94° C. for 30 s
   3. 40° C. for 120 s
   4. 72° C. for 10 s
   5. 94° C. for 15 s
   6. 40° C. for 30 s
   7. 72° C. for 20 s+3 s/cycle
   8. Cycle to step 5, 25 times
   9. Hold at 4° C.
   2. Recovery Reaction—PCR Conditions (Generic Protocol)
   Reaction mix (total volume=50 μl):
1× Reaction buffer (Pfx or Proofstart)
5-10 μl assembly reaction mix
0.3-0.75 mM dNTPs
50 pmol primer (5′ terminal primer, sense orientation)
50 pmol primer (3′ terminal primer, anti-sense orientation)
DNA polymerase (Pfx or Proofstart, 2.5-5U)
+/−1 mM MgSO₄
+/−1× enhancer solution (Pfx enhancer or Proofstart buffer Q)
   1. 94° C. 120 s (Proofstart only)
   2. 94° C. 45 s
   3. 60° C. 30 s
   4. 72° C. 120 s
   5. Cycle to step 2, 25 times
   6. 72° C. 240 s
   7. Hold at 4° C.
   Sequence SEQ ID NO:20

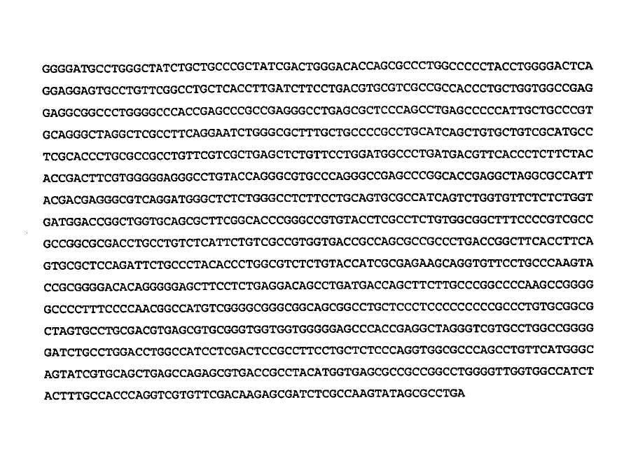

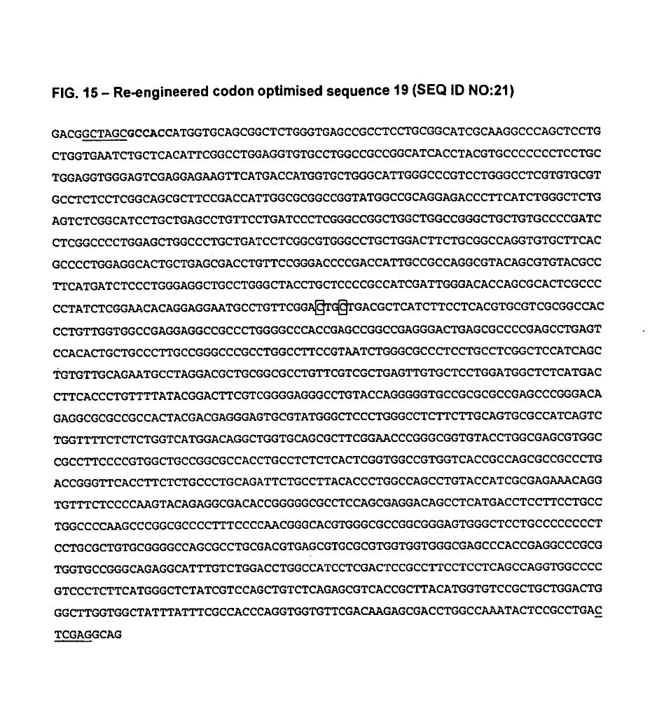

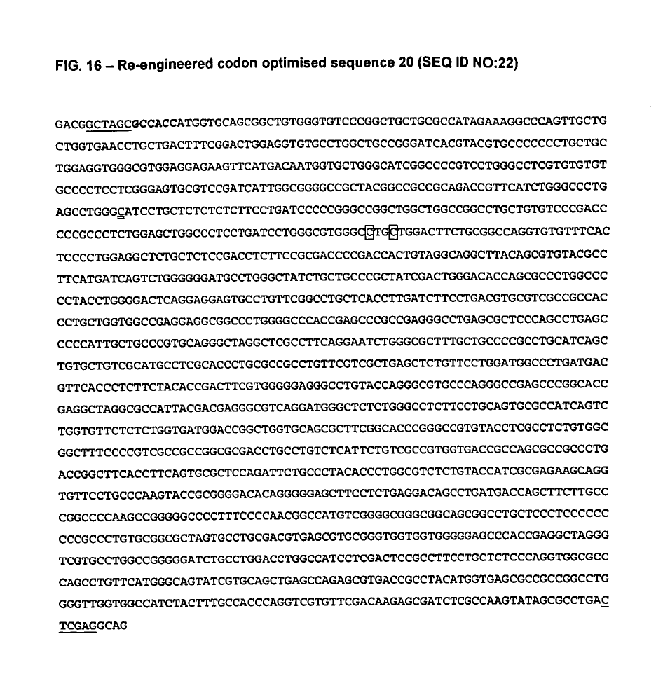

Although SEQ ID NO: 20 has a very good CI index (0.755), it was noticed that it contained a doublet of rare codons at amino acids position 131 and 132. These codons were manually substituted with more frequent codons by changing the DNA sequence from TTGTTG to CTGCTG. To facilitate cloning, an internal BamHI site was removed by mutating G to C (see the double-underlined nucleotide in FIG. 16). To facilitate cloning and expression, restriction enzyme sites and a Kozak sequence were added. The final engineered sequence (SEQ ID NO:22) is shown in FIG. 16. The Syngene programme was used to fragment this sequence into oligonucleotides with a minimum overlap of 19-20 bases.

FIG. 16 therefore shows the re-engineered P501S codon optimised sequence 20 (SEQ ID NO:22). Restriction enzyme sites are underlined, Kozak sequence is bolded, re-engineered DNA sequence to remove a rare codon doublet is boxed and a silent point mutation to remove a BamHI site is double-underlined.

Using a similar two-step PCR protocol to the one described above, full-length P501S fragment was amplified and cloned into pVAC. Positive clones were identified by restriction enzyme analysis and confirmed by double-stranded sequencing. This generates plasmid JNW764. The sequence of the P501S coding cassette is shown in FIG. 16 (SEQ ID NO: 22).

DNA Sequence Similarity

Pair distances following alignment by the ClustalV (weighted) method are shown in Table 3 below. Table 4 below shows percent similarity between the starting human P501S sequence and the two codon optimised sequences SEQ ID NO:21 and 22 selected for further investigation. The data confirms that the codon optimised DNA sequences are approximately 80% similar to the original P501S sequence.

TABLE 4

| SEQ ID NO: | % similarity with starting P501S sequence |
|---|---|
| 21 | 79.6 |
| 22 | 79.4 |

### EXAMPLE VII

### Codon-Optimised CPC Sequences

1.—Approach

Since the original CPC sequence was originally designed for optimal expression in yeast, this section describes the process of codon optimising for human expression.

2.—Sequence Design

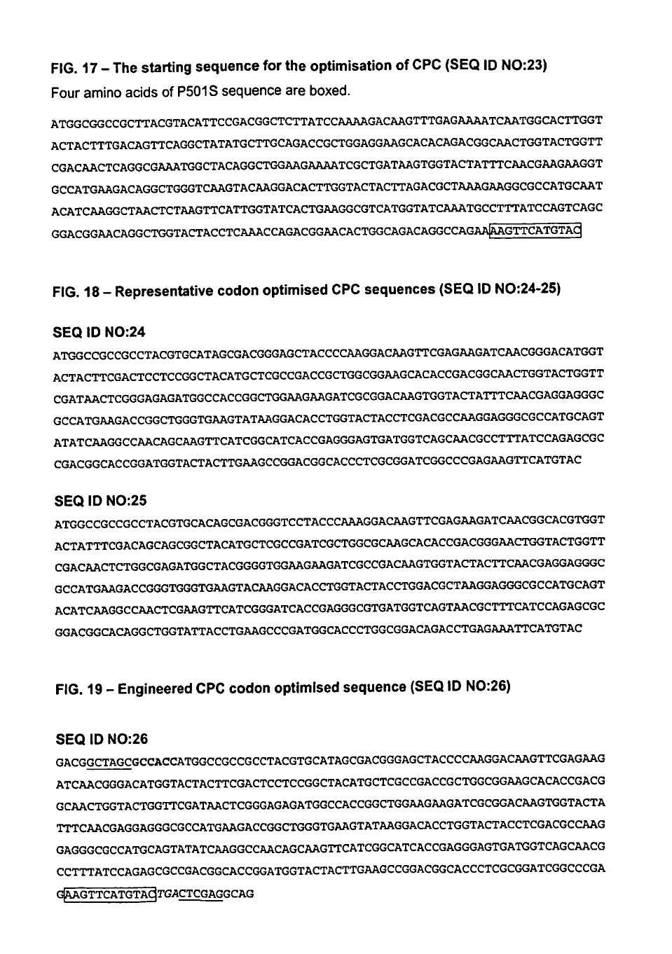

The starting sequence for the optimisation of CPC is shown in FIG. 17 (SEQ ID NO: 23). This is derived entirely from the pRIT15201 and contains the entire coding sequence of CPC plus four amino acids of P501S to facilitate downstream cloning. Using the Syngene programme, a selection of codon optimised sequences were obtained, from which representative sequences are shown in FIG. 18 (SEQ ID NO: 24-25). Table 5 below shows a comparison of the codon coefficient index for the starting CPC sequence and the two representative codon optimised sequences.

TABLE 5
Codon coefficient indices for two CPC optimised sequences

| Sequence | Codon coefficient index (CI) |
|---|---|
| Original CPC = SEQ ID NO: 23 | 0.506 |
| SEQ ID NO: 24 | 0.809 |
| SEQ ID NO: 25 | 0.800 |

In addition to the codon optimisation, all sequences were also screened for restriction enzyme cloning sites. On the basis of the highest CI value and a favourable restriction enzyme site profile, SEQ ID NO: 24 was selected for construction. To facilitate cloning and expression, 5′ and 3′ cloning sites were added and a Kozak sequence (GCCACC) was inserted 5′ of the initiating ATG start codon. This engineered sequence is shown in FIG. 19 (SEQ ID NO:26). This sequence includes four amino aicds of P501S (boxed), restriction enzyme cloning sites (NheI and XhoI, underlined), a Kozak sequence (Bold), a stop codon (italicised) and 4 bp of flanking irrelevant DNA to facilitate cloning.

The Syngene programme was used to fragment this sequence into 50-60-mer oligonucleotides with a minimum overlap of 18-20 bases.

Using a similar two-step PCR protocol to the one described above, the correct fragment was recovered/amplified and cloned into pVAC. Positive clones were identified by restriction enzyme analysis and sequence verified generating vector JNW759.

4.—DNA Similarity

Pair Distances following alignment ClustalV (Weighted) are shown in Table 6 below. The table shows percent similarity at the DNA level between the starting sequence of CPC and the codon optimised sequence and confirms that the codon optimised sequences are approximately 80% similar to the original CPC sequence.

TABLE 6

| | % similarity with |
|---|---|
| Sequence SEQ ID NO: | starting CPC sequence |
| 24 | 80.2 |
| 25 | 81.6 |

### EXAMPLE VII

### Construction of the P501S Fusion Candidate

All the candidates shown in the schematic below are codon optimised and constructed using overlapping PCR methodologies from plasmids JNW764 and JNW759 as templates (SEQ ID NO: 22 and SEQ ID NO: 26 respectively), and cloned into the expression vector p7313 ie.

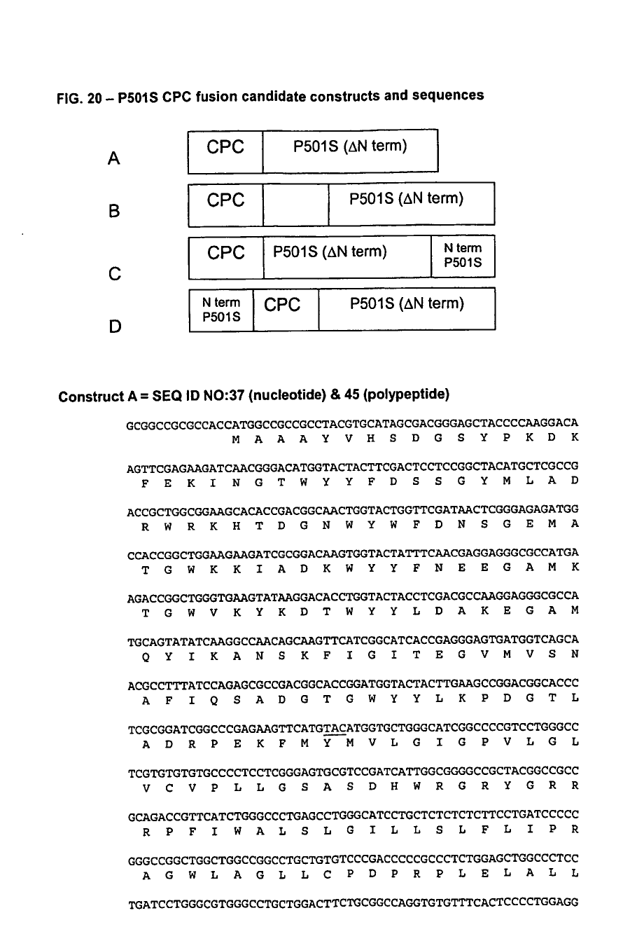

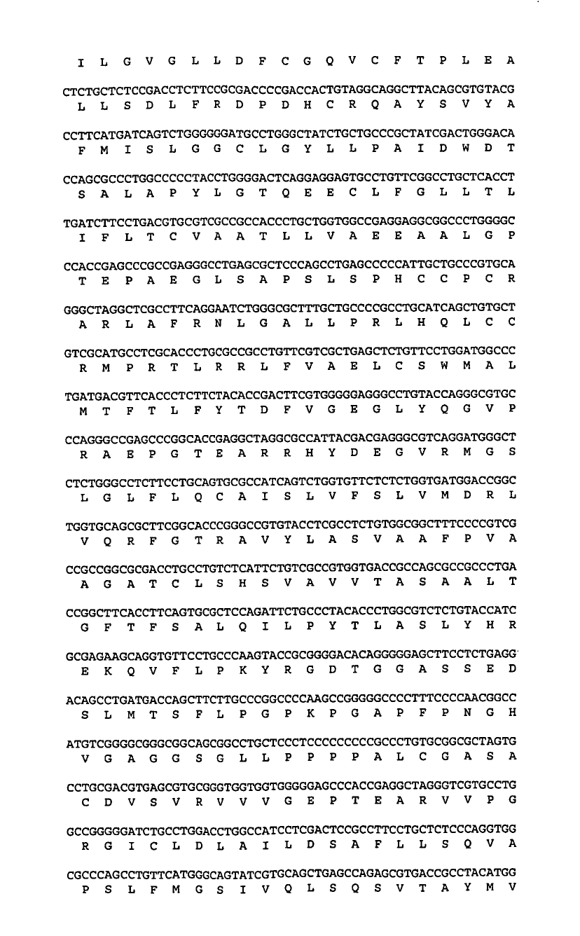

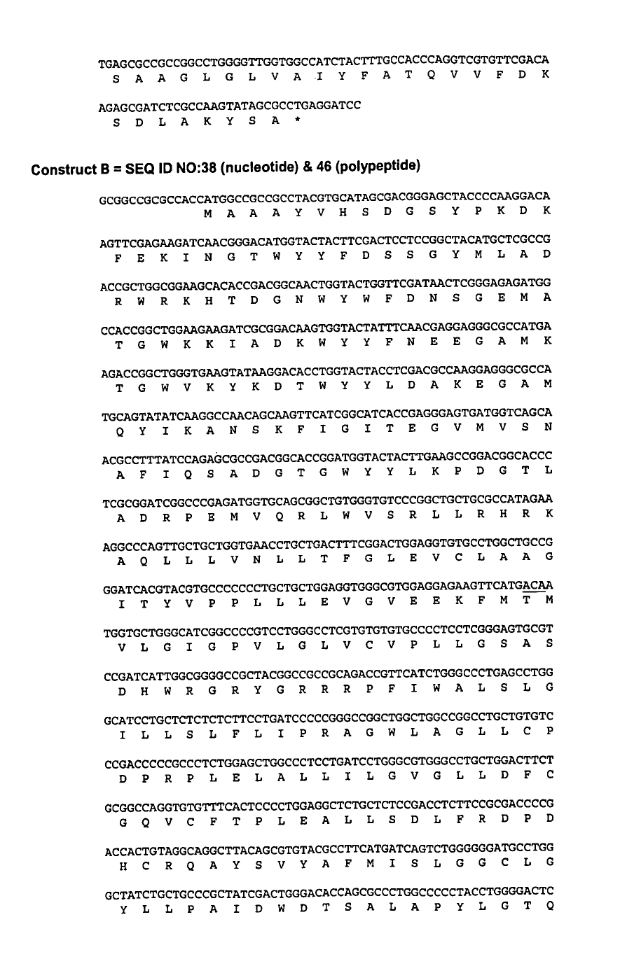

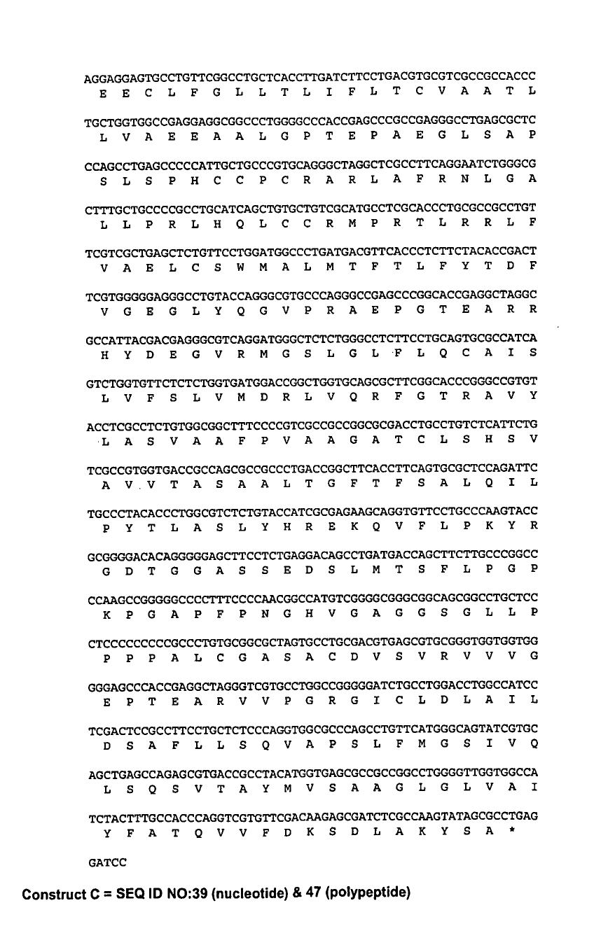

The four candidates shown schematically below are based upon CPC-P501S. Codon optimised CPC-P501S is construct A. Candidates B, C, D also include the sequence encoding the N terminal 50 amino acids of P501S, positioned either at the N terminus of CPC-P501S (construct D), the C terminus of CPC-P501S (construct C), or between CPC and P501S (construct B). A schematic representation of the constructs is given in FIG. 20.

The nucleotide and protein sequence for each of the four constructs is shown in SEQ ID NO: 37-40 for the nucleotide sequences, and SEQ ID NO. 45-48 for the corresponding polypeptide sequences. In constructs A, C and D, the underlined codon preferentially encodes tyrosine (either TAC or TAT) but the nucleotide sequence may be altered to encode threonine (either ACA, ACC, ACG or ACT). In construct B, the underlined codon preferentially encodes threonine (either ACA, ACC, ACG or ACT), but the nucleotide sequence may be altered to encode tyrosine (either TAC or TAT). In all constructs, the coding sequence is flanked by appropriate restriction enzyme cloning sites (in this case, NotI and BamHI), and a Kozak sequence immediately upstream of the initiating ATG. Table 7 below shows the plasmid identification for the constructs detailed above:

TABLE 7

| | Amino acid at | | |
|---|---|---|---|
| Construct | underlined codon | Sequence of codon | Plasmid ID |
| A | Tyrosine | TAC | JNW771 |
| B | Threonine | ACA | JNW773 |
| B | Tyrosine | TAC | JNW770 |
| C | Tyrosine | TAC | JNW777 |
| D | Tyrosine | TAC | JNW769 |

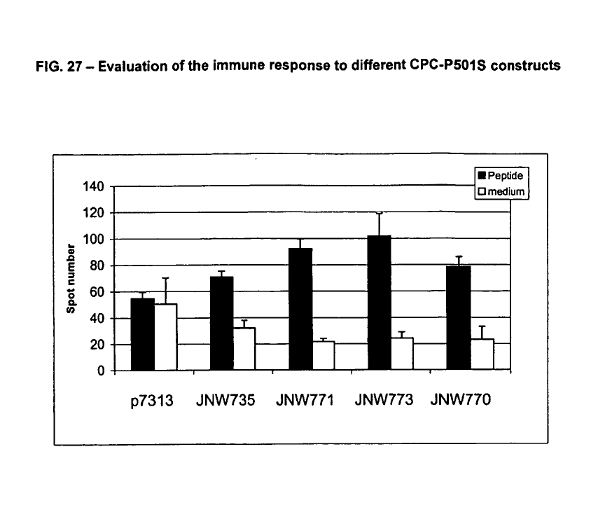

The cellular responses following immunisation with p7313-ie (empty vector), pVAC-P501S (JNW735), JNW770, JNW771 and JNW773 were assessed by ELISPOT following a primary immunisation by PMID at day 0 and three boosts at day 21, 42 and 70. Assays were carried out 7 days post boost. FIG. 27 shows that good IL-2 ELISPOT responses were detected in mice immunised with JNW770, JNW771 and JNW773.

### EXAMPLE IX

### Immunogenicity Experiments Using Particle-Mediated Intra-Dermal Delivery (PMID) Studies

Full-length P501S, when delivered by particle mediated intra-dermal delivery (PMID), generates good antibody & cellular responses. These data demonstrate that the PMID is a very effective delivery route. Furthermore, comparison of P501S and CPC-P501S confirms that CPC-P501S induces a stronger immune response as determined by peptide ELISPOT.

1.—Materials & Methods

1.1. Cutaneous Gene Gun Immunisation

Plasmid DNA was precipitated onto 2 μm diameter gold beads using calcium chloride and spermidine. Loaded beads were coated onto Tefzel tubing as described (Eisenbraum et al, 1993; Pertmer et al, 1996). Particle bombardment was performed using the Accell gene delivery system (PCT WO 95/19799). For each plasmid, female C57BL/6 mice were immunised on days 0, 21, 42 and 70. Each administration consisted of two bombardments with DNA/gold, providing a total dose of approximately 4-5 μg of plasmid.

1.2. ELISPOT Assays for T Cell Responses to the P501S Gene Product

a) Preparation of Splenocytes

Spleens were obtained from immunised animals at 7-14 days post boost. Spleens were processed by grinding between glass slides to produce a cell suspension. Red blood cells were lysed by ammonium chloride treatment and debris was removed to leave a fine suspension of splenocytes. Cells were resuspended at a concentration of 8×10⁶/ml in RPMI complete media for use in ELISPOT assays.

b) Screening of Peptide Library

A peptide library covering a majority of the P501S sequence was obtained from Corixa Corp. The library contained fifty 15-20mer peptides overlapping by 4-11 amino acids peptides. The peptides are numbered 1-50. In addition, a prediction programme (H-G. Rammensee, et al.: Immunogenetics, 1999, 50: 213-219) (http://syfpeithi.bmi-heidelberg.com/) was used to predict putative Kb and Db epitopes from the P501S sequence. The ten best epitopes for Kb and Db were ordered from Mimotopes (UK) and included in the library (peptides 51-70). For screening of the peptide library, peptides were used at a final concentration of 50 μg/ml (approx. 25-50 μM) in IFNγ and IL-2 ELISPOTS using the protocol described below. For IFNγ ELISPOTS, IL-2 was added to the assays at 10 ng/ml. Splenocytes used for the screening were taken at day 84 from C57BL/6 mice immunised at day 0, 21, 42 and 70. Three peptides were identified from the library screen—Peptides 18 (HCRQAYSVYAFMISLGGCLG), 22 (GLSAPSLSPHCCPCRARLAF) and 48 (VCLAAGITYVPPLLLEVGV). These peptides were subsequently used in the ELISPOT assays

c) ELISPOT Assay

Plates were coated with 15 μg/ml (in PBS) rat anti mouse IFNγ or rat anti mouse IL-2 (Pharmingen). Plates were coated overnight at +4° C. Before use the plates were washed three times with PBS. Splenocytes were added to the plates at 4×10⁵ cells/well. Peptides identified in the library screen were re-ordered from Genemed Synthesis and used at a final concentration of 50 μg/ml. CPC-P501S protein (GSKBio) was used in the assay at 20 μg/ml. ELISPOT assays were carried out in the presence of either IL-2 (10 ng/ml), IL-7 (10 ng/ml) or no cytokine. Total volume in each well was 200 μl. Plates containing peptide stimulated cells were incubated for 16 hours in a humidified 37° C. incubator.

e) Development of ELISPOT Assay Plates.

Cells were removed from the plates by washing once with water (with 10 minute soak to ensure lysis of cells) and three times with PBS. Biotin conjugated rat anti mouse IFNg or IL-2 (Phamingen) was added at 1 μg/ml in PBS. Plates were incubated with shaking for 2 hours at room temperature. Plates were then washed three times with PBS before addition of Streptavidin alkaline phosphatase (Caltag) at 1/1000 dilution. Following three washes in PBS spots were revealed by incubation with BCICP substrate (Biorad) for 15-45 mins. Substrate was washed off using water and plates were allowed to dry. Spots were enumerated using an image analysis system devised by Brian Hayes, Asthma Cell Biology unit, GSK.

1.3. ELISA Assay for Antibodies to the P501S Gene Product

Serum samples were obtained from the animals by venepuncture on days −1, 28, 49 and 56, and assayed for the presence of anti-P501S antibodies. ELISA was performed using Nunc Maxisorp plates coated overnight at 4° C. with 0.5 μg/ml of CPC-P501S protein (GSKBio) in sodium bicarbonate buffer. After washing with TBS-Tween (Tris-buffered saline, pH 7.4 containing 0.05% of Tween 20) the plates were blocked with Blocking buffer (3% BSA in TBS-Tween buffer) for 2 hrs at room temperature. All sera were incubated at 1:100 dilution for 1 hr at RT in Blocking buffer. Antibody binding was detected using HRP-conjugated rabbit anti-mouse immunoglobulins (#P0260, Dako) at 1:2000 dilution in Blocking buffer. Plates were washed again and bound conjugate detected using Fast OPD colour reagents (Sigma, UK). The reaction was stopped by the addition of 3M sulphuric acid, and the OPD product quantitated by measuring the absorbance at 490 nm.

1.4. Transient Transfection Assays

Human P501S expression from various DNA constructs was analysed by transient transfection of the plasmids into CHO (Chinese hamster ovary) cells followed by Western blotting on total cell protein. Transient transfections were performed with the Transfectam reagent (Promega) according to the manufacturer's guidelines. In brief, 24-well tissue culture plates were seeded with 5×10⁴ CHO cells per well in 1 ml DMEM complete medium (DMEM, 10% FCS, 2 mM L-glutamine, penicillin 100 IU/ml, streptomycin 10 μg/ml) and incubated for 16 hours at 37° C. 0.5 μg DNA was added to 25 μl of 0.3M NaCl (sufficient for one well) and 2 μl of Transfectam was added to 25 μl of Milli-Q. The DNA and Transfectam solutions were mixed gently and incubated at room temperature for 15 minutes. During this incubation step, the cells were washed once in PBS and covered with 150 μl of serum free medium (DMEM, 2 mM L-glutamine). The DNA-Transfectam solution was added drop wise to the cells, the plate gentle shaken and incubated at 37° C. for 4-6 hours. 500 μl of DMEM complete medium was added and the cells incubated for a further 48-72 hours at 37° C.

2. Western Blot Analysis of CHO Cells Transiently Transfected with P501S Plasmids

The transiently transfected CHO cells were washed with PBS and treated with a Versene (1:5000)/0.025% trypsin solution to transfer the cells into suspension. Following trypsinisation, the CHO cells were pelleted and resuspended in 50 μl of PBS. An equal volume of 2×NP40 lysis buffer was added and the cells incubated on ice for 30 minutes. 100 μl of 2× TRIS-Glycine SDS sample buffer (Invitrogen) containing 50 mM DTT was added and the solution heated to 95° C. for 5 minutes. 1-20 μl of sample was loaded onto a 4-20% TRIS-Glycine Gel 1.5 mm (Invitrogen) and electrophoresed at constant voltage (125V) for 90 minutes in 1× TRIS-Glycine buffer (Invitrogen). A pre-stained broad range marker (New England Biolabs, #P7708S) was used to size the samples. Following electrophoresis, the samples were transferred to Immobilon-P PVDF membrane (Millipore), pre-wetted in methanol, using an Xcell III Blot Module (Invitrogen), 1× Transfer buffer (Invitrogen) containing 20% methanol and a constant voltage of 25V for 90 minutes. The membrane was blocked overnight at 4° C. in TBS-Tween (Tris-buffered saline, pH 7.4 containing 0.05% of Tween 20) containing 3% dried skimmed milk (Marvel). The primary antibody (10E3) was diluted 1:1000 and incubated with the membrane for 1 hour at room temperature. Following extensive washing in TBS-Tween, the secondary antibody (HRP-conjugated rabbit anti-mouse immunoglobulins (#P0260, Dako)) was diluted 1:2000 in TBS-Tween containing 3% dried skimmed milk and incubated with the membrane for one hour at room temperature. Following extensive washing, the membrane was incubated with Supersignal West Pico Chemiluminescent substrate (Pierce) for 5 minutes. Excess liquid was removed and the membrane sealed between two sheets of cling film, and exposed to Hyperfilm ECL film (Amersham-PharmaciaBiotech) for 1-30 minutes.

3. Generation of the Full-Length Human P501S Expression Cassette

The starting point for the construction of a P501S expression cassette was the plasmid pcDNA3.1-P501S (Corixa Corp), which has a pcDNA3.1 backbone (Invitrogen) containing a full-length human P501S cDNA cassette cloned between the EcoRI and NotI sites. This vector is also termed JNW673. The presence of P501S was confirmed by fluorescent sequencing. The sequence of the cDNA cassette is given by the NCBI/Genbank sequence (accession number AY033593). Human P501S was PCR amplified from JNW673 template DNA, restricted with XbaI and SalI and cloned into the NheI/XhoI sites of pVAC generating vector JNW680. The correct orientation of the fragment relative to the CMV promoter was confirmed by PCR and by DNA sequencing. The sequence of the expression cassette is shown in FIG. 12 (SEQ ID NO: 17).

To construct a CPC-P501S expression cassette, CPC-P501S was PCR amplified from the vector pRIT15201 (see FIG. 7), restricted with XbaI and SalI and cloned into the NheI and XhoI sites of pVAC, generating plasmid JNW735. The correct orientation was confirmed by PCR and sequencing. The sequence of the CPC-P501S expression cassette is shown in FIG. 13 (SEQ ID NO:18).

4. Expression of Human P501S from Plasmids JNW680 and JNW735

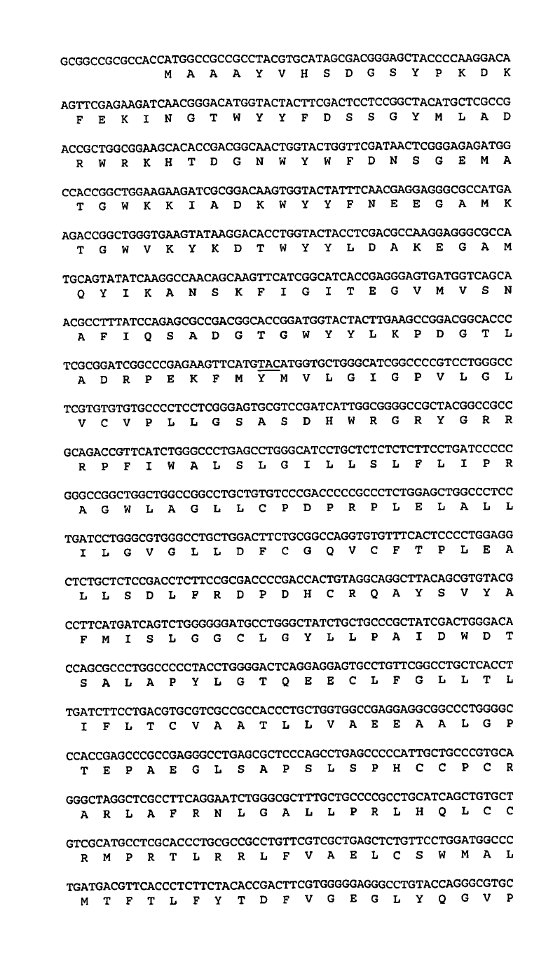

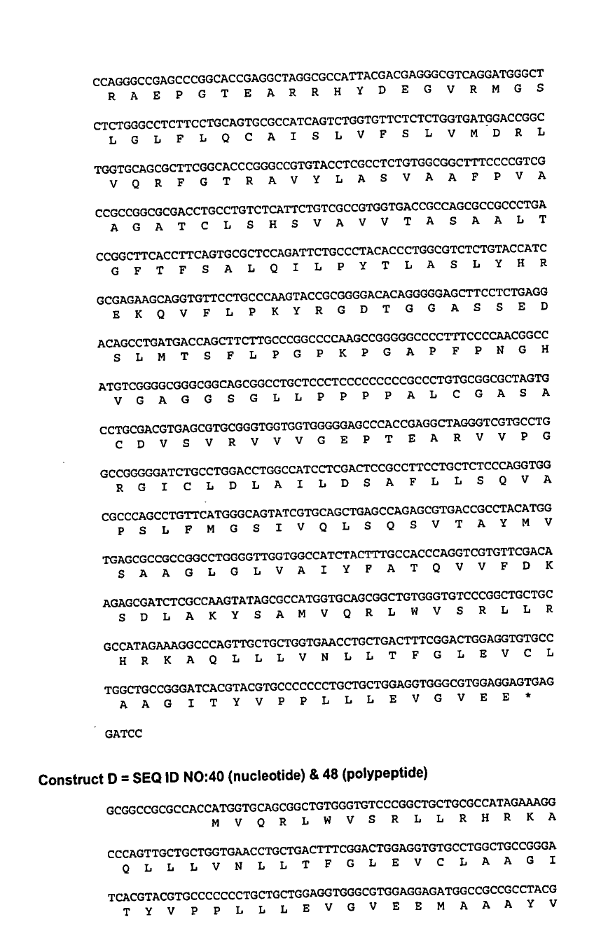

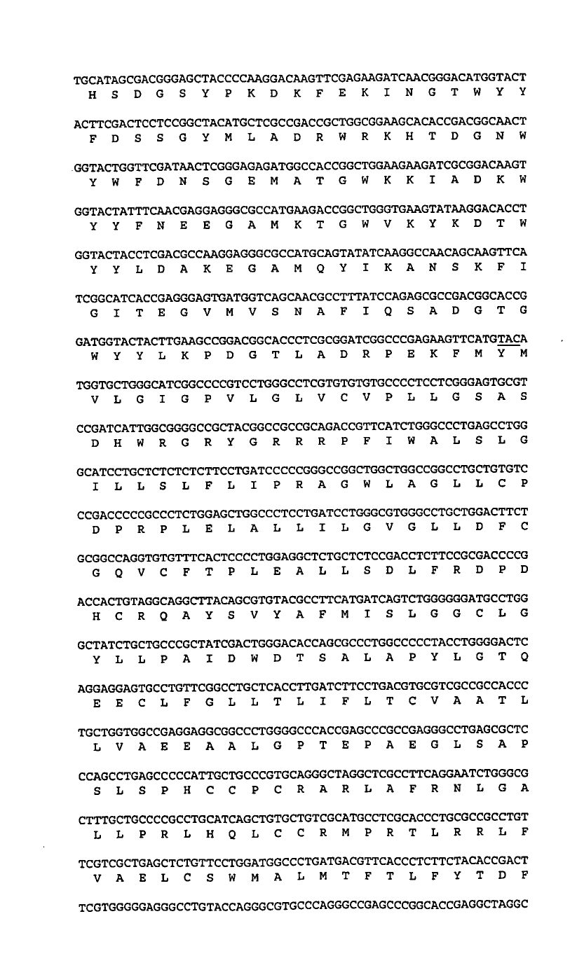

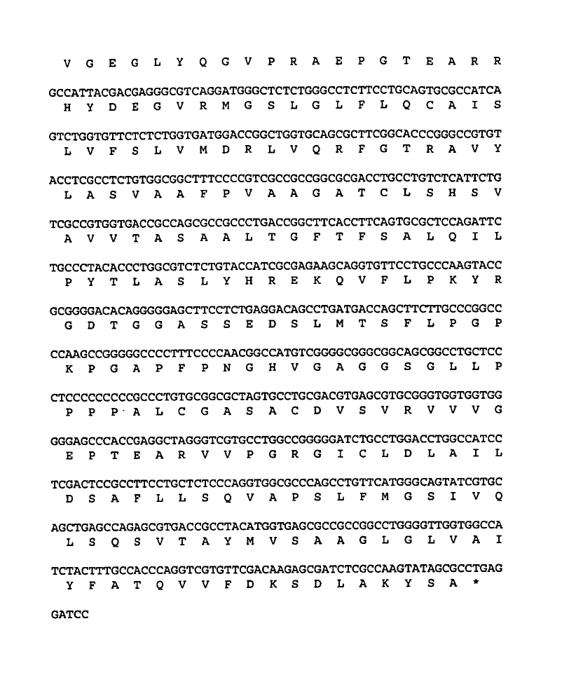

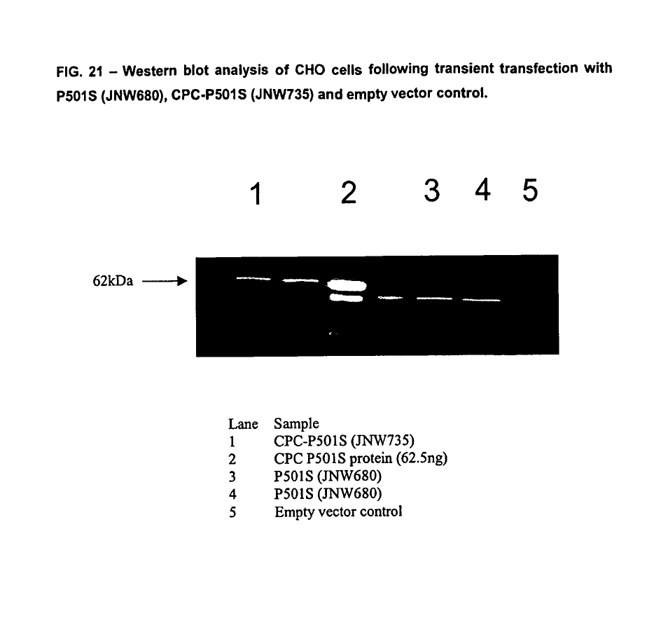

The P501S expression plasmids were transiently transfected into CHO cells and a total cell lysate prepared as described in methods. A Western blot of a total cell lysate identified single bands of approximately 55 kDa and 62 kDa for samples transfected with JNW680 and JNW735 respectively (FIG. 21). This is consistent with the predicted molecular weights of 59.3 kDa and 63.3 kDa for P501S and CPC-P501S respectively. The addition of the CPC tag does not adversely affect the expression of P501S.

5. Results

5.1. Antibody Responses to Human P501S Following PMID Immunisation

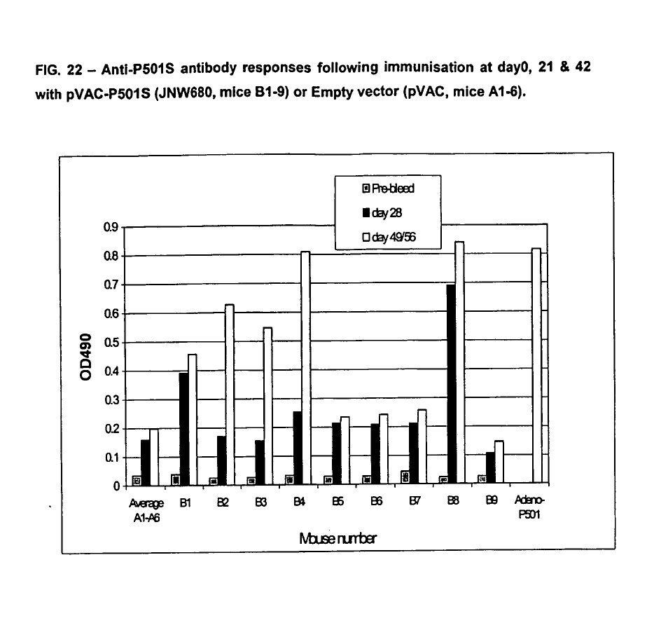

The antibody responses following immunisation with pVAC (empty vector) and pVAC-P501S (JNW680) were assessed by ELISA following a primary immunisation by PMID at day 0 and three boosts at day 21 and day 42 and day 70. FIG. 22 shows the antibody responses from sera taken at day −1, day 28 and day 49 (mice A1-3, B1-3) and day 56 (mice A4-6, B4-9). Whilst there were some non-specific responses to the pVAC empty vector, specific responses to the P501S construct were seen in 5 of 9 mice.

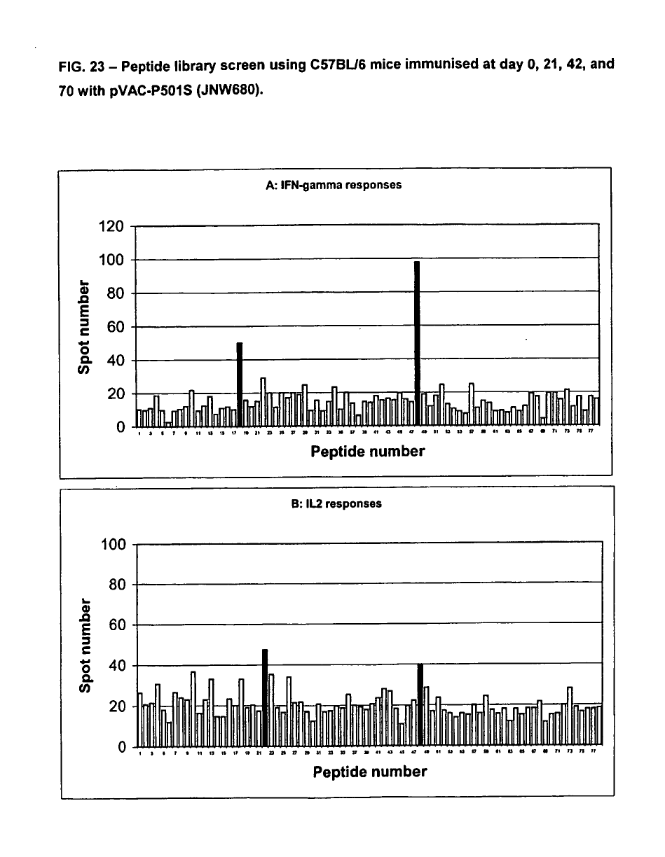

5.2. Identification of novel T cell epitopes from human P501S in C57BL/6 mice by screening of a P501S peptide library Following immunisation with JNW680 (pVAC-P501S) by PMID at day 0 and three boosts at day 21 and day 42 and day 70, ELISPOT assays were carried out at day 84. Peptides from the P501S library were tested at 50 μg/ml final concentration. From this initial screen, three peptides were found to stimulate IFNγ and/or IL-2 secretion. Peptides 18, 22 and 48 (FIG. 23). These peptides were used in subsequent cellular assays.

5.3. Cellular Responses to pVAC-P501S (JNW680) Following PMID Immunisation

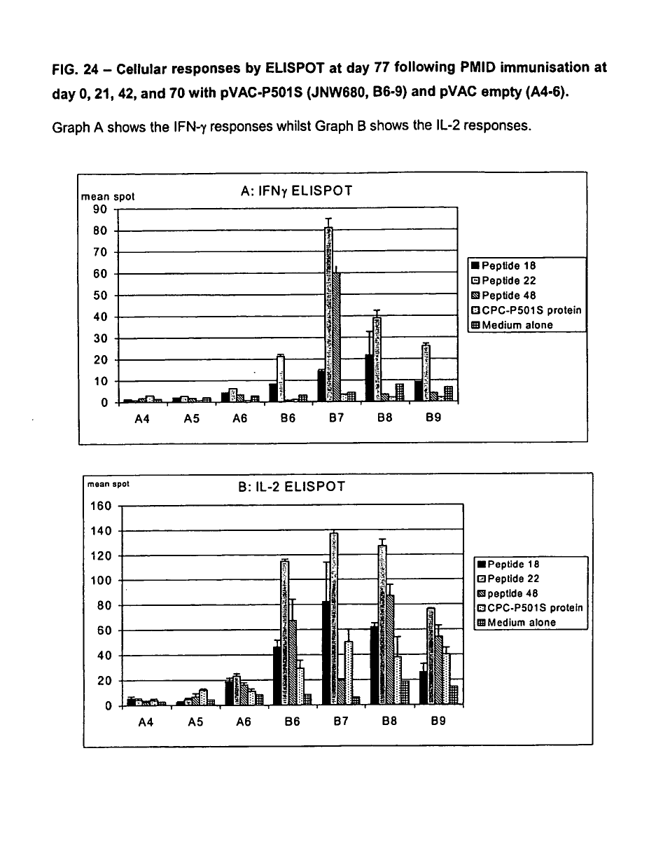

The cellular responses following immunisation with pVAC (empty vector) and pVAC-P501S were assessed by ELISPOT following a primary immunisation by PMID at day 0 and three boosts at day 21, 42 and 70. Assays were carried out 7 days post boost. Two different assay conditions were used: 1) Peptides 18, 22 and 48 identified in the peptide library screen used at 50 μg/ml final concentration and 2) CPC-P501S protein used at 20 μg/ml final concentration. FIG. 24A shows that whilst there were no P501S-specific responses to the empty vector (A4-6), the pVAC-P501S construct induced specific IFN-γ responses to Peptides 18 and 22 in all mice (B6-9) whilst one mouse (B7) also showed an IFN-γ response to Peptide 48. FIG. 24B shows that all mice showed specific IL-2 responses to Peptides 18, 22 and 48. Furthermore, pVAC-P501S immunised mice (B6-9) also showed moderate IL-2 responses to CPC-P501S, whereas the empty vector immunised mice (A4-6) showed no responses.

5.4. Comparison of Cellular Responses to P501S and CPC-P501S Following PMID Immunisation.

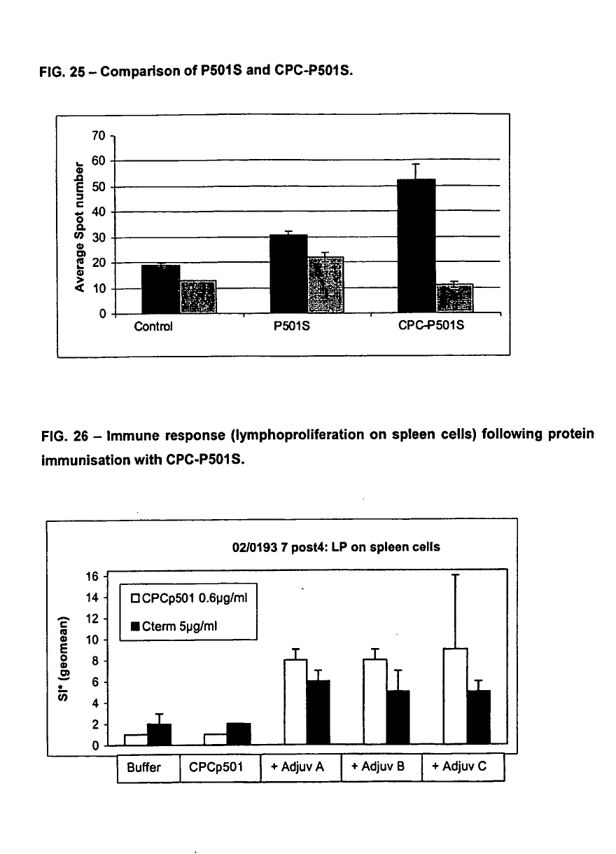

The cellular responses following immunisation with pVAC (empty vector), pVAC-P501S (JNW680) and CPC-P501S (JNW735) were assessed by ELISPOT following a primary immunisation by PMID at day 0 and boosts at day 21 and 42. Assays were carried out 7 days post boost. Two different assay conditions were used: 1) Peptides 18, 22 and 48 identified in the peptide library screen used at 50 μg/ml final concentration and 2) CPC-P501S protein used at 20 μg/ml final concentration. FIG. 25 shows that at day 28, CPC-P501S induced good IL-2 responses to 10 μg/ml of peptide 22, whilst there were no P501S-specific responses to either the empty vector or the pVAC-P501S. These results were also seen using CPC-P501S protein to re-stimulated the splenocytes. At day 49 (post 2^{nd} boost), the responses induced by P501S and CPC-P501S were equivalent. These data suggest that the addition of the CPC tag improves the kinetics and/or magnitude of the response to P501S.

### EXAMPLE IX

### Immunogenicity Experiments in Mice Using P501S Protein+Adjuvant Studies

1. Design and Adjuvant Formulation

The immune response induced by vaccination using the recombinant purified CPC-P501S protein formulated in adjuvants is characterized in experiments performed in mice. Groups of 5 to 10, eight weeks old female C57BL6 mice are vaccinated, 2-6 times intra-muscularly at 2 weeks intervals with 10 μg of the CPC-P501S protein formulated in different adjuvant systems. The volume administered corresponds to 1/10^{th} of a human dose (50 μl).

The serology (total Ig response) and cellular response (T cell lymphoproliferation and cytokine production) are analyzed on spleen cells, 6-14 days after the last vaccination using standard protocols as described in Gerard, c. et al, 2001, Vaccine 19, 2583-2589.

The data of one representative experiment is shown. It included 5 groups of eight C57BL/6 female mice which received 4 intramuscular injections of CPC P501 (10 μg)+adjuvant (A, B, C) at days 0, 14, 28, 42. Example V provides an experimental protocol of how to carry out the formulations. Briefly the adjuvant formulations are as follows (quantities given for one dose of 100 μl)):
Adjuvant A: QS21 (10 μg), MPL (10 μg) and CpG7909 (100 μg) made according to the method disclosed in WO 00/62800;
Adjuvant B: formulation of QS21 (20 μg), MPL (20 μg), CpG7909 (100 μg) and 50 μf SB62 oil-in-water emulsion (WO 95/17210);
Adjuvant C: formulation of QS21 (10 μg), MPL (10 μg), CpG7909 (100 μg) and 10 μl SB62 oil-in-water emulsion (WO 99/12565).
   2. Serology

The total Ig response induced by vaccination was measured by ELISA using either the CPC-P501 or RA12-P501 (C term, which is a truncated form of the P501 protein corresponding to the C terminus of the protein fused at its N terminus, to a TB derived protein RA12-Ra12 is derived from MTB32A antigen described in Skeiky et al., Infection and Immun. (1999) 67:3998-4007).

The adjuvanted CPC-P501S proteins give a good antibody response after vaccination.

3. Cellular Response

3.1. Lymphoproliferation

7 days after the latest vaccine, lymphoproliferation was performed on spleen cells individually. 2.10e5 spleen cells were plated in quadruplicate, in 96 well microplate, in RPMI medium containing 1% normal mice serum. After 72 hours of restimulation with either the immunogen (CPC-P501) or the truncated protein (RA12 P501) at different concentration, 1 μCi 3H thymidine (Amersham 5 Ci/ml) was added. After 16 hours, cells were harvested onto filter plates. Incorporated radioactivity was counted in a β counter. Results are expressed in CPM or as stimulation indexes* (geomean CPM in cultures with antigen/geomean CPM in cultures without antigen).

Re-stimulation with ConA (2 μg/ml) as positive control was included as positive control.

As shown in FIG. 26, a P501 specific lymphoproliferation is seen in the spleen of all groups of mice receiving the adjuvanted protein after in vitro re-stimulation with either the immunogen or another P501 protein made in another expression system (E. coli), indicating that T cells have been primed in vivo by the vaccination.

3.2. IFNg Production Measured by Intracellular Staining of Spleen Cells

Bone Marrow Dendritic Cells (BMDC) obtained after culture of mouse PBL for 7 days in the presence of GMCSF.

7 days after the latest vaccine, spleen or PBL are collected and a cell suspension prepared. 10e6 cells (1 pool per group) were incubated +/−18 hrs with 10e5 BMDC pulsed overnight with 10 μg/ml of either the CPCp501 protein or the RA12.

After a treatment with the 2.4.G.2 antibody, spleen cells were stained with fluorescent anti CD4 and CD8 antibodies (anti CD4-APC and an anti CD8PerCP). After a permeabilization and fixation step, cells were stained with a fluorescent anti IFNg-FITC antibody.

In mice vaccinated with CPC P501 in different adjuvant, both CD4 and CD8 T cells are shown to produce IFNg in response to DC pulsed with either the immunogen and the C-term p501 made in E. coli (as shown by intracellular straining of spleen and PBLs). There is an increase of 4-10× in the % of cells making this cytokine in the groups receiving the adjuvanted CPC-P501S compared to the protein alone, and between 0.1 to 10% of CD4 or CD8 T cells are shown to produce IFNg.

In conclusion, these data allow to conclude that the adjuvanted CPC-P501 protein is immunogenic in mice.

Both a P501 specific humoral and cellular responses including IFNg production by CD4 and CD8 T cells can be detected after several intramuscular vaccination with CPC P501 in adjuvants.

### EXAMPLE X

### CPC-MUC-1 Constructs and Sequences

CPC sequence is taken from nucleotide SEQ ID NO. 28.

MUC1 sequence is available from Genbank database (accession number NM_{—}002456).

1. MUC1-CPC Construct

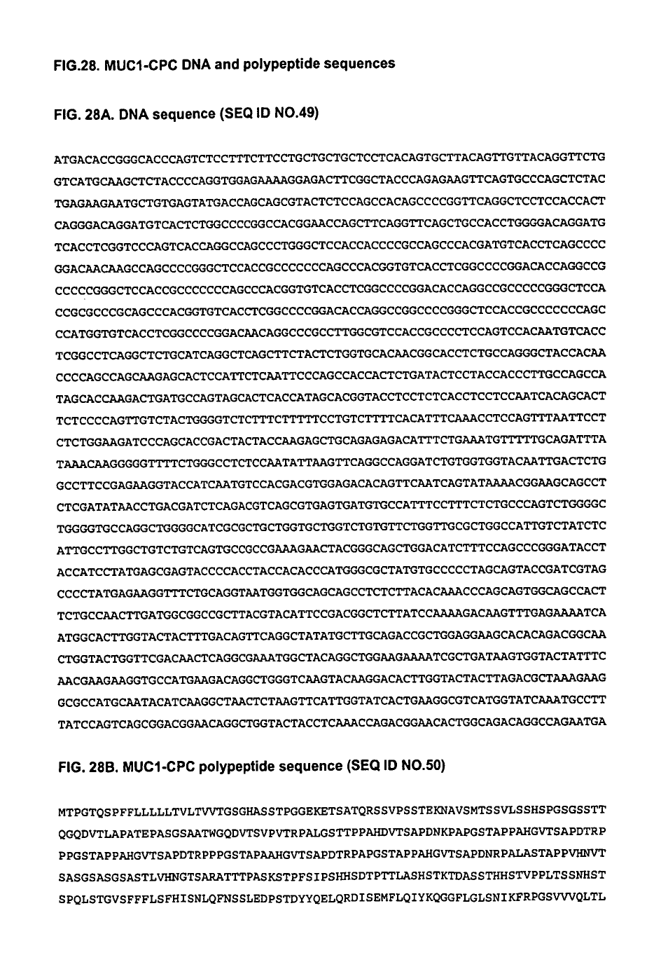

Due to the presence of a signal sequence in MUC1 that is cleaved post-translationally, the CPC motif was placed at the C-terminus. The resulting MUC1-CPC DNA sequence is depicted in SEQ ID NO. xx (FIG. 28A) and the corresponding MUC1-CPC protein sequence in SEQ ID NO. yy (FIG. 28B).

2. ss-CPC-MUC1 Construct

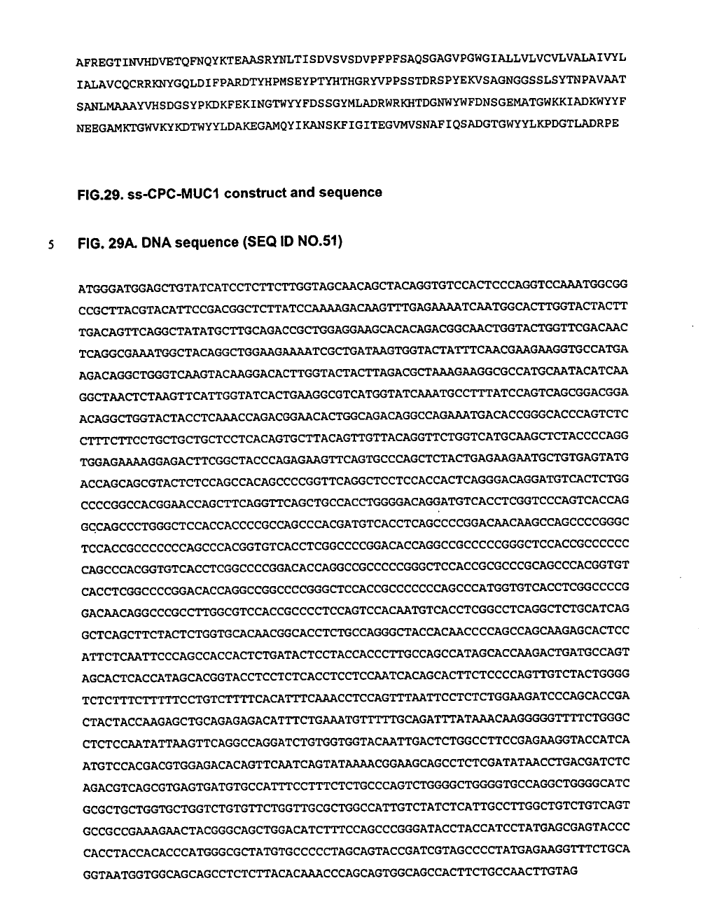

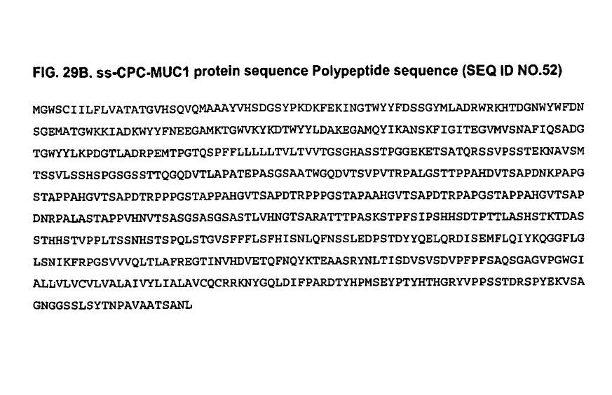

Due to the presence of a signal sequence in MUC1 that is cleaved post-translationally, the MUC1 signal sequence was replaced by a heterologous leader sequence (from the human immunoglobulin heavy chain) and the CPC motif was inserted between the heterologous leader sequence and the MUC1 sequence, generating a sequence termed ss-CPC-MUC1 as depicted in FIG. 29.

1. A fusion partner protein comprising a choline binding domain and a heterologous promiscuous T helper epitope.

2. A fusion partner protein according to claim 1 wherein the choline binding domain is derived from the C terminus of LytA.

3. A fusion partner protein according to claim 2 wherein the C-LytA or derivatives comprises at least four repeats of any of SEQ ID NO: 1 to 6.

4. A fusion partner protein according to claim 1, wherein the choline binding domain is selected from the group of:
a) the C-terminal domain of LytA as set forth in SEQ ID NO:7;
b) the sequence of SEQ ID NO:8;
c) a peptide sequence comprising an amino acid sequence having at least 85% identity to any of SEQ ID NO:1 to 6; and
d) a peptide sequence comprising an amino acid sequence having at least 15, 20, 30, 40, 50 or 100 contiguous amino acids from the amino acid sequence of SEQ ID NO:7 or SEQ ID NO:8.

5. A fusion partner protein as claimed in claim 1 further comprising a heterologous protein.

6. A fusion protein as claimed in claim 5 wherein the heterologous protein is chemically conjugated the fusion partner.

7. A fusion protein as claimed in claim 5 wherein the heterologous protein is derived from an organism selected from the following group: Human Immunodeficiency virus HIV-1, human herpes simplex viruses, cytomegalovirus, Rotavirus, Epstein Barr virus, Varicella Zoster Virus, hepatitis A virus, hepatitis C virus, hepatitis E virus, from Respiratory Syncytial virus, parainfluenza virus, measles virus, mumps virus, human papilloma viruses, flaviviruses, and Influenza virus, from Neisseria spp, Moraxella spp, Bordetella spp; Mycobacterium spp., M. tuberculosis; Escherichia spp, enterotoxic E. coli; Salmonella spp,; Listeria spp; Helicobacter spp; Staphylococcus spp., S. aureus, S. epidermidis; Borrelia spp; Chlamydia spp., C. trachomatis, C. pneumoniae; Plasmodium spp., P. falciparum; Toxoplasma spp., or Candida spp.

8. A fusion protein as claimed in claim 5 wherein the heterologous protein is a tumour associated protein or tissue specific protein or immunogenic fragment thereof.

9. A fusion protein as claimed in claim 8 wherein the heterologous protein or fragment thereof is selected from MAGE 1, MAGE 3, MAGE 4, PRAME, BAGE, LAGE 1, LAGE 2, SAGE, HAGE, XAGE, PSA, PAP, PSCA, prostein, P501S, HASH2, Cripto, B726, NY-BR1.1, P510, MUC-1, Prostase, STEAP, tyrosinase, telomerase, survivin, CASB616, P53, or her 2 neu.

10. A fusion protein as claimed in claim 6 further comprising an affinity tag of at least 4 histidine residues.

11. A nucleic acid sequence encoding a protein of claim 1.

12. An expression vector comprising a nucleic acid sequence of claim 11.

13. A host cell transformed with an expression vector of claim 12.

14. An immunogenic composition comprising a protein as claimed in any of claim 1 and a pharmaceutically acceptable excipient.

15. An immunogenic composition as claimed in claim 14 which additionally comprises a TH-1 inducing adjuvant.

16. An immunogenic composition as claimed in claim 15 in which the TH-1 inducing adjuvant is selected from the group of adjuvants comprising: 3D-MPL, QS21, a mixture of QS21 and cholesterol, a CpG oligonucleotide or a mixture of two or more said adjuvants.

17. A process for the preparation of a immunogenic composition, comprising admixing the fusion protein of claim 6 with a suitable adjuvant, diluent or other pharmaceutically acceptable carrier.

18. A process for producing a fusion protein of claim 1 comprising culturing a host cell comprising a vector encoding said fusion protein under conditions sufficient for the production of said fusion protein and recovering the fusion protein from the culture medium.

19. A pharmaceutical composition comprising a fusion protein of claim 1.

20. (canceled)

21. (canceled)

22. (canceled)

23. (canceled)

24. (canceled)

25. (canceled)

26. A method of treating a patient suffering from cancer by administrating a safe and effective amount of a composition according to claim 12.

27. A method according to claim 26 wherein said cancer is prostate cancer, colorectal cancer, lung cancer, breast cancer or melanoma.

28. An immunogenic composition comprising a DNA sequence as claimed in claim 11 and a pharmaceutically acceptable excipient.

29. A process for the preparation of an immunogenic composition, comprising admixing the fusion protein of a polynucleotide of claim 11 with a suitable adjuvant, diluent or other pharmaceutically acceptable carrier.

30. A method of eliciting an immune response in a patient comprising administering an immunogenic composition of claim 14.

31. The method according to claim 30, wherein said immune response is to be elicited by sequential administration of i) the said protein followed by a nucleic acid encoding said protein; or ii) a nucleic acid encoding said protein followed by said protein.

32. The method according to claim 31 wherein said nucleic acid sequence is coated onto biodegradable beads or delivered via a particle bombardment approach.

33. The method according to claim 31 wherein said protein is adjuvanted.

34. The method according to claim 31 wherein the patient is suffering from or susceptible to cancer.

35. The method according to claim 34 wherein said cancer is prostate cancer, colon cancer, lung cancer, breast cancer or melanoma.
